SEQUENCE LISTING (100 sequences)

SEQ ID NO: 1 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 442 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIISFFLALTSTALLFLLFFLTLRFSVVKRGKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 442 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDFWVLVVVGGVLACYSLLVTVAFIIFWVRSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLIALVTSGALLAVLGITGYFLMNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLLAALLALLAALLALLAALLARSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 428 aa, Artificial Sequence
  Synthetic polypeptide
MALPVTALLLPLALLLHAARPQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 435 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 477 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 391 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEPSEGSAPGSPAGSPTIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 435 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 477 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 437 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDMALIVLGGVAGLLLFIGLGIFFCVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDMALIVLGGVAGLLLFIGLGIFFCVRRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLCYILDAILFLYGIVLTLLYCRLKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLLLILLGVLAGVLATLAALLARSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDITLGLLVAGVLVLLVSLGVAIHLCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVSFCLVMVLLFAVDTGLYFSVKTNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVAAILGLGLVLGLLGPLAILLALYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLCYLLDGILFIYGVILTALFLRVKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 441 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVMSVATIVIVDICITGGLLLLVYYWSKNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDGFLFAEIVSIFVLAVGVYFIAGQDKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 437 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDGIIVTDVIATLLLALGVFCFAGHETKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 435 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVIGFRILLLKVAGFNLLMTLRLWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 438 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIIVAVVIATAVAAIVAAVVALIYCRKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVLFYLAVGIMFLVNTVLWVTIRKEKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIIILVGTAVIAMFFWLLLVIILRTKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 432 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLGWLCLLLLPIPLIVWVKRKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIAIYCIGVFLIACMVVTVILCRMKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 476 aa, Artificial Sequence
  Synthetic polypeptide
MALPVTALLLPLALLLHAARPQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 623 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 510 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 403 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 421 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEAFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 406 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 406 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 421 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 451 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 451 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEPSEGSAPGSPAGSPTSTEEGTSTEPSEGSAIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 421 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 406 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 433 aa, Artificial Sequence
  Synthetic polypeptide
MLRLLLALNLFPSIQVTGGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 436 aa, Artificial Sequence
  Synthetic polypeptide
METDTLLLWVLLLWVPGSTGDGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 431 aa, Artificial Sequence
  Synthetic polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 432 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 442 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 452 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQLGLHIWQLRSQCMWPRETQLLLEVPPSTEDARSCQFPEEERGERSAEEKGRLGDLWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 454 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCCVKRRKPRGDVVKVIVSVQRKRQEAEGEATVIEALQAPPDVTTVAVEETIPSFTGRSPNHRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 442 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKKYFFKKEVQQLSVSFSSLQIKALQNAVEKEVQAEDNIYIENSLYATDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 453 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCYKVGFFKRNLKEKMEAGRGVPNGIPAEDSEQLASGQEAGDPGCLKPLHEKDSESGGGKDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 510 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 365 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDPQLCYILDAILFLYGIVLTLLYCRLKIQVRKAAITSYEKSDGVYTGLSTRNQETYETLKHEKPPQKRGRKKLLYIFKQPFMRPVQTTQEEDGCSCRFPEEEEGGCEL

SEQ ID NO: 56 - protein, 366 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRLKIQVRKAAITSYEKSDGVYTGLSTRNQETYETLKHEKPPQ

SEQ ID NO: 57 - protein, 432 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 390 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 483 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 472 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 477 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 474 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 433 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 442 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 435 aa, Artificial Sequence; identical to SEQ ID NO: 7
  Synthetic polypeptide

SEQ ID NO: 66 - protein, 428 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 431 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 469 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 391 aa, Artificial Sequence; identical to SEQ ID NO: 10
  Synthetic polypeptide

SEQ ID NO: 70 - protein, 397 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 383 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 386 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 395 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 394 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 438 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 419 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 409 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 393 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 436 aa, Artificial Sequence; identical to SEQ ID NO: 1
  Synthetic polypeptide

SEQ ID NO: 80 - protein, 429 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 332 aa, Artificial Sequence
  Synthetic polypeptide
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 82 - protein, 500 aa, Artificial Sequence
  Synthetic polypeptide
MPPAVGGPVGYTPPDGGWGWAVVIGAFISIGFSYAFPKSITVFFKEIEGIFHATTSEVSWISSIMLAVMYGGGPISSILVNKYGSRIVMIVGGCLSGCGLIAASFCNTVQQLYVCIGVIGGLGLAFNLNPALTMIGKYFYKRRPLANGLAMAGSPVFLCTLAPLNQVFFGIFGWRGSFLILGGLLLNCCVAGALMRPIGPKPTKAGKDKSKASLEKAGKSGVKKDLHDANTDLIGRHPKQEKRSVFQTINQFLDLTLFTHRGFLLYLSGNVIMFFGLFAPLVFLSSYGKSQHYSSEKSAFLLSILAFVDMVARPSMGLVANTKPIRPRIQYFFAASVVANGVCHMLAPLSTTYVGFCVYAGFFGFAFGWLSSVLFETLMDLVGPQRFSSAVGLVTIVECCPVLLGPPLLGRLNDMYGDYKYTYWACGVVLIISGIYLFIGMGINYRLLAKEQKANEQKKESKEEETSIDVAGKPNEVTKAAESPDQKDTDGGPKEEESPV

SEQ ID NO: 83 - protein, 478 aa, Artificial Sequence
  Synthetic polypeptide
MPPMPSAPPVHPPPDGGWGWIVVGAAFISIGFSYAFPKAVTVFFKEIQQIFHTTYSEIAWISSIMLAVMYAGGPVSSVLVNKYGSRPVVIAGGLLCCLGMVLASFSSSVVQLYLTMGFITGLGLAFNLQPALTIIGKYFYRKRPMANGLAMAGSPVFLSSLAPFNQYLFNTFGWKGSFLILGSLLLNACVAGSLMRPLGPNQTTSKSKNKTGKTEDDSSPKKIKTKKSTWEKVNKYLDFSLFKHRGFLIYLSGNVIMFLGFFAPIIFLAPYAKDQGIDEYSAAFLLSVMAFVDMFARPSVGLIANSKYIRPRIQYFFSFAIMFNGVCHLLCPLAQDYTSLVLYAVFFGLGFGSVSSVLFETLMDLVGAPRFSSAVGLVTIVECGPVLLGPPLAGKLVDLTGEYKYMYMSCGAIVVAASVWLLIGNAINYRLLAKERKEENARQKTRESEPLSKSKHSEDVNVKVSNAQSVTSERETNI

SEQ ID NO: 84 - protein, 465 aa, Artificial Sequence
  Synthetic polypeptide
MGGAVVDEGPTGVKAPDGGWGWAVLFGCFVITGFSYAFPKAVSVFFKELIQEFGIGYSDTAWISSILLAMLYGTGPLCSVCVNRFGCRPVMLVGGLFASLGMVAASFCRSIIQVYLTTGVITGLGLALNFQPSLIMLNRYFSKRRPMANGLAAAGSPVFLCALSPLGQLLQDRYGWRGGFLILGGLLLNCCVCAALMRPLVVTAQPGSGPPRPSRRLLDLSVFRDRGFVLYAVAASVMVLGLFVPPVFVVSYAKDLGVPDTKAAFLLTILGFIDIFARPAAGFVAGLGKVRPYSVYLFSFSMFFNGLADLAGSTAGDYGGLVVFCIFFGISYGMVGALQFEVLMAIVGTHKFSSAIGLVLLMEAVAVLVGPPSGGKLLDATHVYMYVFILAGAEVLTSSLILLLGNFFCIRKKPKEPQPEVAAAEEEKLHKPPADSGVDLREVEHFLKAEPEKNGEVVHTPETSV

SEQ ID NO: 85 - protein, 456 aa, Artificial Sequence
  Synthetic polypeptide
MRLARLLRGAALAGPGPGLRAAGFSRSFSSDSGSSPASERGVPGQVDFYARFSPSPLSMKQFLDFGSVNACEKTSFMFLRQELPVRLANIMKEISLLPDNLLRTPSVQLVQSWYIQSLQELLDFKDKSAEDAKAIYERPRRTWLQVSSLCCMACKMIFTDTVIRIRNRHNDVIPTMAQGVIEYKESFGVDPVTSQNVQYFLDRFYMSRISIRMLLNQHSLLFGGKGKGSPSHRKHIGSINPNCNVLEVIKDGYENARRLCDLYYINSPELELEELNAKSPGQPIQVVYVPSHLYHMVFELFKNAMRATMEHHANRGVYPPIQVHVTLGNEDLTVKMSDRGGGVPLRKIDRLFNYMYSTAPRPRVETSRAVPLAGFGYGLPISRLYAQYFQGDLKLYSLEGYGTDAVIYIKALSTDSIERLPVYNKAAWKHYNTNHEADDWCVPSREPKDMTTFRSA

SEQ ID NO: 86 - protein, 254 aa, Artificial Sequence
  Synthetic polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 87 - protein, 254 aa, Artificial Sequence
  Synthetic polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 88 - protein, 300 aa, Artificial Sequence
  Synthetic polypeptide
  MISC_FEATURE (16)..(300): these amino acids may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 89 - protein, 15 aa, Artificial Sequence
  Synthetic polypeptide
GGGGSGGGGSGGGGS

SEQ ID NO: 90 - protein, 30 aa, Artificial Sequence
  Synthetic polypeptide
GGGGSGGGGSGGGGSGGGGSGGGGSGGGGS

SEQ ID NO: 91 - protein, 45 aa, Artificial Sequence
  Synthetic polypeptide
GGGGSGGGGSGGGGSGGGGSGGGGSGGGGSGGGGSGGGGSGGGGS

SEQ ID NO: 92 - protein, 60 aa, Artificial Sequence
  Synthetic polypeptide
GGGGSGGGGSGGGGSGGGGSGGGGSGGGGSGGGGSGGGGSGGGGSGGGGSGGGGSGGGGS

SEQ ID NO: 93 - protein, 75 aa, Artificial Sequence
  Synthetic polypeptide
GGGGSGGGGSGGGGSGGGGSGGGGSGGGGSGGGGSGGGGSGGGGSGGGGSGGGGSGGGGSGGGGSGGGGSGGGGS

SEQ ID NO: 94 - protein, 150 aa, Artificial Sequence
  Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 95 - protein, 225 aa, Artificial Sequence
  Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 96 - protein, 300 aa, Artificial Sequence; identical to SEQ ID NO: 88
  Synthetic polypeptide

SEQ ID NO: 97 - protein, 487 aa, Artificial Sequence
  Synthetic polypeptide
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 483 aa, Artificial Sequence
  Synthetic polypeptide
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 19 aa, Artificial Sequence
  Synthetic polypeptide
ATNFSLLKQAGDVEENPGP

SEQ ID NO: 100 - protein, 18 aa, Artificial Sequence
  Synthetic polypeptide
ATNFSLLKQAGDVEENPG

(19) United States
(12) Patent Application Publication
(10) Pub. No.: US 2021/0340219 A1
(43) Pub. Date: Nov. 4, 2021

(54) CHIMERIC RECEPTOR POLYPEPTIDES IN COMBINATION WITH TRANS METABOLISM MOLECULES MODULATING INTRACELLULAR LACTATE CONCENTRATIONS AND THERAPEUTIC USES THEREOF

(71) Applicant: Sotio, LLC, Boston, MA (US)

(72) Inventors: Kathleen McGinness, Cambridge, MA (US); Seth Ettenberg, Cambridge, MA (US); Luke Barron, Cambridge, MA (US); Michael Fray, Cambridge, MA (US); Charles Wilson, Cambridge, MA (US); Gregory Motz, Cambridge, MA (US)

(73) Assignee: Sotio, LLC, Boston, MA (US)

(21) Appl. No.: 17/274,021

(22) PCT Filed: Sep. 6, 2019
(86) PCT No.: PCT/US2019/050013

§ 371 Date: Mar. 5, 2021

Related U.S. Application Data
(60) Provisional application No. 62/728,338, filed on Sep. 7, 2018.
(60) Provisional application No. 62/728,306, filed on Sep. 7, 2018.

(51) Int. Cl. C07K 14/71; C12N 5/0783; C07K 14/47; C07K 14/705; C07K 14/725; C07K 14/735; C12N 15/86; C12N 7/00; A61K 35/17; A61K 38/17; A61P 35/00; C07K 16/30; A61K 39/395

(52) U.S. Cl. CPC C07K 14/71; A61K 2039/505; C07K 14/4702; C07K 14/70521; C07K 14/70578; C07K 14/7051; C07K 14/70535; C07K 14/70517; C12N 15/86; C12N 7/00; A61K 35/17; A61K 38/1774; A61K 38/179; A61K 38/177; A61K 38/1709; A61P 35/00; C07K 16/303; A61K 39/39558; C07K 2319/30; C07K 2319/33; C07K 2319/03; C07K 2319/02; C07K 2317/622; C07K 2317/569; C12N 5/0636

(57) ABSTRACT

Disclosed herein are genetically engineered hematopoietic cells, which express one or more lactate-modulating factors (e.g., polypeptides), and optionally a chimeric receptor polypeptide (e.g., an antibody-coupled T cell receptor (ACTR) polypeptide or a chimeric antigen receptor (CAR) polypeptide) capable of binding to a target antigen of interest. Also disclosed herein are uses of the engineered hematopoietic cells for inhibiting cells expressing a target antigen in a subject in need thereof.

### CROSS REFERENCE TO RELATED APPLICATIONS

This application claims the benefit of the filing dates of U.S. Provisional Application No. 62/728,338, filed Sep. 7, 2018, and U.S. Provisional Application No. 62/728,306, filed Sep. 7, 2018. The entire contents of each of the prior applications are incorporated by reference herein.

### BACKGROUND OF DISCLOSURE

Cancer immunotherapy, including cell-based therapy, is used to provoke immune responses attacking tumor cells while sparing normal tissues. It is a promising option for treating various types of cancer because of its potential to evade genetic and cellular mechanisms of drug resistance, and to target tumor cells while sparing normal tissues.

Cell-based therapy may involve cytotoxic T cells having reactivity skewed toward cancer cells. Eshhar et al., Proc. Natl. Acad. Sci. U.S.A.; 1993; 90(2):720-724; Geiger et al., J Immunol. 1999; 162(10):5931-5939; Brentjens et al., Nat. Med. 2003; 9(3):279-286; Cooper et al., Blood. 2003; 101(4):1637-1644; and Imai et al., Leukemia. 2004; 18:676-684. One approach is to express a chimeric receptor having an antigen-binding domain fused to one or more T cell activation signaling domains. Binding of a cancer antigen via the antigen-binding domain results in T cell activation and triggers cytotoxicity. Recent results of clinical trials with infusions of chimeric receptor-expressing autologous T lymphocytes provided compelling evidence of their clinical potential. Pule et al., Nat. Med. 2008; 14(11):1264-1270; Porter et al., N Engl J Med; 2011; 25;365(8):725-733; Brentjens et al., Blood. 2011; 118(18):4817-4828; Till et al., Blood. 2012; 119(17):3940-3950; Kochenderfer et al., Blood. 2012; 119(12):2709-2720; and Brentjens et al., Sci Transl Med. 2013; 5(177):177ra138.

Another approach is to express an antibody-coupled T cell Receptor (ACTR) protein in a hematopoietic cell (e.g., a hematopoietic stem cell, an immune cell, such as an NK cell or a T cell), the ACTR protein containing an extracellular Fc-binding domain. When the ACTR-expressing hematopoietic cells (e.g., ACTR-expressing T cells, also called “ACTR T cells”) are administered to a subject together with an anti-cancer antibody, they may enhance toxicity against cancer cells targeted by the antibody via their binding to the Fc domain of the antibody. Kudo et al., Cancer Research. (2014) 74:93-103.

Cell-based immune therapies, while promising, have faced challenges caused by specific characteristics of the tumor microenvironment (TME), which is cellular environment created via the interaction between malignant tumor cells and non-transformed cells. It is therefore of great importance to develop strategies to improve efficacy of cell-based immune therapies in light of the TME.

### SUMMARY OF DISCLOSURE

The present disclosure is based on the development of strategies to modulate the intracellular lactate concentration in hematopoietic cells such as hematopoietic stem cells (HSCs) or immune cells, including those that express a chimeric receptor polypeptide, such as an antibody-coupled T-cells receptor (ACTR) polypeptide or a chimeric antigen receptor (CAR) polypeptide, for use in cell-based immune therapy. Modulation of the intracellular lactate concentration may be achieved by expressing (e.g., over-expressing) in hematopoietic cells (e.g., HSCs or immune cells such as T cells or natural killer cells) one or more lactate-modulating factors such as lactate-modulating polypeptides, e.g., those described herein. Such genetically engineered hematopoietic cells (e.g., immune cells) are expected to have an enhanced metabolic activity relative to native hematopoietic cells of the same type (e.g., immune cells of the same type), for example, in a low glucose environment, a low-amino acid environment, a low pH environment, and/or a hypoxic environment (e.g., in a tumor microenvironment). Such genetically engineered immune cells may also have modulated epigenetic states (e.g., acetylation states) and/or modulated levels of immunosuppressive metabolites (e.g., kynurenine). As such, hematopoieic cells such as HSCs or immune cells that co-express one or more lactate-modulating factors (e.g., polypeptides) and a chimeric receptor polypeptide would exhibit superior bioactivities (e.g., under tumor microenvironment such as low glucose, low amino acid, low pH, and/or hypoxic conditions, optionally in the presence of a therapeutic antibody), for example, cell proliferation, activation (e.g., increased cytokine production, e.g., IL-2 or IFNγ production), cytotoxicity, and/or in vivo anti-tumor activity.

Accordingly, provided herein are modified (e.g., genetically modified) hematopoietic cells (e.g., hematopoietic stem cells, or immune cells such as T cells or natural killer cells) that have the capacity to have altered intracellular regulation of lactate concentrations relative to the wild-type immune cells of the same type. In some instances, the modified immune cells may express or overly express a lactate-modulating factor, for example, a lactate-modulating polypeptide. The lactate-modulating polypeptide may be an enzyme involved in lactate synthesis (for example, LDHA, which catalyzes the interconversion of lactate and pyruvate), a lactate transporter (for example, MCT), or a polypeptide that inhibits a pathway that competes for lactate-synthesis substrates (for example, PDK1). Exemplary lactate-modulating polypeptides include, but are not limited to, L-lactate dehydrogenase A chain (LDHA), Monocarboxylate transporter 1 (MCT1), Monocarboxylate transporter 2 (MCT2), Monocarboxylate transporter 4 (MCT4), and pyruvate dehydrogenase kinase 1 (PDK1).

The modified immune cells may further express a chimeric receptor polypeptide, which may comprise (a) an extracellular target binding domain; (b) a transmembrane domain; and (c) a cytoplasmic signaling domain (e.g., a cytoplasmic domain that comprises an immunoreceptor tyrosine-based activation motif (ITAM)). In some embodiments, the chimeric receptor polypeptide is an antibody-coupled T cell receptor (ACTR), which comprises an extracellular Fc-binding domain (a). In other embodiments, the chimeric receptor is a chimeric antigen receptor (CAR), which comprises an extracellular antigen binding domain (a). In some examples, (c) is located at the C-terminus of the chimeric receptor polypeptide. In some instances, the chimeric polypeptide may further comprise at least one co-stimulatory signaling domain. In other instances, the chimeric receptor polypeptide may be free of co-stimulatory signaling domains.

Any of the chimeric receptor polypeptides described herein (e.g., an ACTR polypeptide or a CAR polypeptide) may further comprise a hinge domain, which is located at the C-terminus of (a) and the N-terminus of (b). In other examples, the chimeric receptor polypeptide may be free of any hinge domain. In yet other examples, the chimeric receptor polypeptide, for example, an ACTR polypeptide, may be free of a hinge domain from any non-CD16A receptor. Alternatively or in addition, the chimeric receptor polypeptide further comprises a signal peptide at its N-terminus.

In some embodiments, the chimeric receptor polypeptide disclosed herein may be an ACTR polypeptide comprising an Fc binding domain (a). In some examples, the Fc binding domain of (a) can be an extracellular ligand-binding domain of an Fc-receptor, for example, an extracellular ligand-binding domain of an Fc-gamma receptor, an Fc-alpha receptor, or an Fc-epsilon receptor. In particular examples, the Fc binding domain is an extracellular ligand-binding domain of CD16A (e.g., F158 CD16A or V158 CD16A), CD32A, or CD64A. In other examples, the Fc binding domain of (a) can be an antibody fragment that binds the Fc portion of an immunoglobulin. For example, the antibody fragment can be a single chain variable fragment (ScFv), a single domain antibody, (e.g., a nanobody). Additionally, the Fc binding domain of (a) can be a naturally-occurring protein that binds the Fc portion of an immunoglobulin or an Fc-binding fragment thereof. For example, the Fc binding domain can be Protein A or Protein G, or an Fc-binding fragment thereof. In further examples, the Fc binding domain of (a) can be a synthetic polypeptide that binds the Fc portion of an immunoglobulin. Examples include, but are not limited to, a Kunitz peptide, a SMIP, an avimer, an affibody, a DARPin, or an anticalin.

In some embodiments, the chimeric receptor polypeptide disclosed herein can be a CAR polypeptide comprising an extracellular antigen binding domain (a). In some examples, the extracellular antigen binding domain of (a) is a single chain antibody fragment that binds to a tumor antigen, a pathogenic antigen, or an immune cell specific to an autoantigen. In certain examples, the tumor antigen is associated with a hematologic tumor. Examples include, but are not limited to, CD19, CD20, CD22, Kappa-chain, CD30, CD123, CD33, LeY, CD138, CD5, BCMA, CD7, CD40, and IL-1RAP. In certain examples, the tumor antigen is associated with a solid tumor. Examples include, but are not limited to, GD2, GPC3, FOLR (e.g., FOLR1 or FOLR2), HER2, EphA2, EFGRVIII, IL13RA2, VEGFR2, ROR1, NKG2D, EpCAM, CEA, Mesothelin, MUC1, CLDN18.2, CD171, CD133, PSCA, cMET, EGFR, PSMA, FAP, CD70, MUC16, L1-CAM, B7H3, and CAIX. In certain examples, the pathogenic antigen is a bacterial antigen, a viral antigen, or a fungal antigen, for example, those described herein.

In some embodiments, the transmembrane domain of (b) in any of the chimeric receptor polypeptide (e.g., ACTR or CAR polypeptide) can be of a single-pass membrane protein, e.g., CD8α, CD8β, 4-1BB, CD28, CD34, CD4, FcεRIγ, CD16A, OX40, CD3ζ, CD3ε, CD3γ, CD3δ, TCRα, CD32, CD64, VEGFR2, FAS, and FGFR2B. Alternatively, the transmembrane domain of (b) can be a non-naturally occurring hydrophobic protein segment.

In some embodiments, the at least one co-stimulatory signaling domain of the chimeric receptor polypeptides described herein (e.g., ACTR or CAR polypeptides), if applicable, can be of a co-stimulatory molecule, which can be 4-1BB, CD28, CD28_{LL}_{_{→}}_{GG} variant, OX40, ICOS, CD27, GITR, ICOS, HVEM, TIM1, LFA1, and CD2. In some examples, the at least one co-stimulatory signaling domains is a CD28 co-stimulatory signaling domain or a 4-1BB co-stimulatory signaling domain. In some instances, the ACTR polypeptide may comprise two co-stimulatory signaling domains. In some instances, one of the co-stimulatory signaling domains is a CD28 co-stimulatory signaling domain; and the other co-stimulatory domain can be a 4-1BB co-stimulatory signaling domain, an OX40 co-stimulatory signaling domain, a CD27 co-stimulatory signaling domain, or an ICOS co-stimulatory signaling domain. Specific examples include, but are not limited to, CD28 and 4-1BB; or CD28_{LL}_{_{→}}_{GG} variant and 4-1BB. Alternatively, any of the chimeric receptor polypeptide may be free of any co-stimulatory signaling domain.

In some embodiments, the cytoplasmic signaling domain of (c) in any of the chimeric receptor polypeptides described herein (e.g., ACTR or CAR polypeptides) can be a cytoplasmic domain of CD3ζ or FcεR1γ.

In some embodiments, the hinge domain of any of the chimeric polypeptides to described herein (e.g., ACTR or CAR polypeptides), when applicable, can be of CD28, CD16A, CD8α, or IgG. In other examples, the hinge domain is a non-naturally occurring peptide. For example, the non-naturally occurring peptide may be an extended recombinant polypeptide (XTEN) or a (Gly₄Ser)ₙ polypeptide, in which n is an integer of 3-12, inclusive. In some examples, the hinge domain is a short segment, which may contain up to 60 amino acid residues.

In specific examples, an ACTR polypeptide as described herein may comprise (i) a CD28 co-stimulatory domain; and (ii) a CD28 transmembrane domain, a CD28 hinge domain, or a combination thereof. For example, the ACTR polypeptide comprises components (a)-(e) as shown in Table 4. In particular examples, the ACTR polypeptide comprises the amino acid sequence selected from SEQ ID NOs: 1-80.

In specific examples, a CAR polypeptide described herein may comprise (i) a CD28 co-stimulatory domain or a 4-1BB co-stimulatory domain; and (ii) a CD28 transmembrane domain, a CD28 hinge domain, or a combination thereof. In further specific examples, a CAR polypeptide described herein may comprise (i) a CD28 co-stimulatory domain or a 4-1BB co-stimulatory domain, (ii) a CD8 transmembrane domain, a CD8 hinge domain, or a combination thereof. For example, the CAR polypeptide may comprise an amino acid sequence selected from SEQ ID NOs: 97 and 98.

The hematopoietic cells described herein, expressing the lactate-modulating factor (e.g., polypeptide) and optionally the chimeric receptor polypeptide, may be a hematopoietic stem cell or a progeny thereof. In some embodiments, the hematopoietic cells can be immune cells such as natural killer cell, monocyte/macrophage, neutrophil, eosinophil, or T cell. The immune cells can be derived from peripheral blood mononuclear cells (PBMC), hematopoietic stem cells (HSCs), or induced pluripotent stem cells (iPSCs). In some examples, the immune cell is a T cell, in which the expression of an endogenous T cell receptor, an endogenous major histocompatibility complex, an endogenous beta-2-microglobulin, or a combination thereof has been inhibited or eliminated.

Any of the hematopoietic cells (e.g., HSCs or immune cells) described herein may comprise a nucleic acid or a nucleic acid set, which collectively comprises: (a) a first nucleotide sequence encoding the lactate-modulating factor (e.g., polypeptide); and optionally (b) a second nucleotide sequence encoding the chimeric antigen receptor (CAR) polypeptide. The nucleic acid or the nucleic acid set is an RNA molecule or a set of RNA molecules. In some instances, the immune cell comprises the nucleic acid, which comprises both the first nucleotide sequence and the second nucleotide sequence. In some embodiments, the coding sequence of the lactate-modulating factor is upstream of that of the CAR polypeptide. In some embodiments, the coding sequence of the CAR polypeptide is upstream of that of the lactate-modulating factor. Such a nucleic acid may further comprise a third nucleotide sequence located between the first nucleotide sequence and the second nucleotide sequence, wherein the third nucleotide sequence encodes a ribosomal skipping site (e.g., a P2A peptide), an internal ribosome entry site (IRES), or a second promoter.

In some examples, the nucleic acid or the nucleic acid set is comprised within a vector or a set of vectors, which can be an expression vector or a set of expression vectors (e.g., viral vectors such as lentiviral vectors or retroviral vectors). A nucleic acid set or a vector set refers to a group of two or more nucleic acid molecules or two or more vectors, each encoding one of the polypeptides of interest (i.e., the lactate-modulating polypeptide and the CAR polypeptide). Any of the nucleic acids described herein is also within the scope of the present disclosure.

In another aspect, the present disclosure provides a pharmaceutical composition, comprising any of the immune cells described herein and a pharmaceutically acceptable carrier.

Moreover, provided herein is a method for inhibiting cells expressing a target antigen (e.g., reducing the number of such cells, blocking cell proliferation, and/or suppressing cell activity) in a subject, the method comprising administering to a subject in need thereof a population of the immune cells described herein, which may co-express the lactate-modulating factor (e.g., polypeptide) and the CAR polypeptide. The subject (e.g., a human patient such as a human patient suffering from a cancer) may have been treated or is being treated with an anti-cancer therapy (e.g., an anti-cancer agent). In some examples, at least some of the cells expressing the target antigen are located in a low-glucose environment, a low-amino acid (e.g., low glutamine) environment, a low-pH environment, and/or a hypoxic environment, for example a tumor microenvironment.

In some examples, the immune cells are autologous. In other examples, the immune cells are allogeneic. In any of the methods described herein, the immune cells can be activated, expanded, or both ex vivo. In some instances, the immune cells comprise T cells, which are activated in the presence of one or more of anti-CD3 antibody, anti-CD28 antibody, IL-2, phytohemoagglutinin, and an engineered artificial stimulatory cell or particle. In other instances, the immune cells comprise natural killer cells, which are activated in the presence of one or more of 4-1BB ligand, anti-4-1BB antibody, IL-15, anti-IL-15 receptor antibody, IL-2, IL-12, IL-21 and K562 cells, an engineered artificial stimulatory cell or particle.

In some examples, the subject to be treated by the methods described herein may be a human patient suffering from a cancer, for example, carcinoma, lymphoma, sarcoma, blastoma, and leukemia. Additional exemplary target cancer includes, but are not limited to, a cancer of B-cell origin, breast cancer, gastric cancer, neuroblastoma, osteosarcoma, lung cancer, skin cancer, prostate cancer, colon cancer, renal cell carcinoma, ovarian cancer, rhabdomyosarcoma, leukemia, mesothelioma, pancreatic cancer, head and neck cancer, retinoblastoma, glioma, glioblastoma, liver cancer, and thyroid cancer. Exemplary cancers of B-cell origin is selected from the group consisting of B-lineage acute lymphoblastic leukemia, B-cell chronic lymphocytic leukemia, and B-cell non-Hodgkin's lymphoma.

Also within the scope of the present disclosure are uses of the genetically engineered immune cells described herein, which co-express a lactate-modulating factor (e.g., polypeptide) and a CAR polypeptide for treating a target disease or disorder such as cancer or an infectious disorder, and uses thereof for manufacturing a medicament for the intended medical treatment.

The details of one or more embodiments of the disclosure are set forth in the description below. Other features or advantages of the present disclosure will be apparent from the detailed description of several embodiments and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a schematic illustration showing intracellular synthesis and metabolism pathways of lactate, as well as lactate exportation and importation. Exemplary strategies for modulating intracellular lactate concentrations include regulation of one or more enzymes involved in lactate synthesis, metabolism, and/or transportation, for example, enhancing interconversion of intracellular lactate and pyruvate by, e.g., overexpression of LDHA and increasing cellular transport of lactate by, e.g., overexpression of MCTs.

FIG. 2 is a graph showing the impact of low glucose concentrations on proliferation of immune cells expressing an anti-GPC3 chimeric antigen receptor in the presence of GPC3-expressing target cells.

FIGS. 3A-3B are graphs showing that co-expression of MCT1 (SEQ ID NO: 82) with CAR (SEQ ID NO: 98) in T cells enhanced cell proliferation relative to CAR (SEQ ID NO: 97) alone under tumor-relevant (1.25 mM; FIG. 3A) and approximate peripheral blood level (10 mM; FIG. 3B) glucose conditions.

FIGS. 4A-4B are graphs showing that co-expression of MCT2 (SEQ ID NO: 83) with CAR (SEQ ID NO: 97) in T cells enhanced cell proliferation relative to CAR (SEQ ID NO: 97) alone under tumor-relevant (1.25 mM; FIG. 4A) and approximate peripheral blood level (10 mM; FIG. 4B) glucose conditions.

FIGS. 5A-5B are graphs showing that co-expression of MCT4 (SEQ ID NO: 84) with CAR (SEQ ID NO: 98) in T cells enhanced cell proliferation relative to CAR (SEQ ID NO: 97) alone under tumor-relevant (1.25 mM; FIG. 5A) and approximate peripheral blood level (10 mM; FIG. 5B) glucose conditions.

FIGS. 6A-6B are graphs showing IL-2 production and proliferation as a function of antibody concentration of T cells expressing an ACTR (SEQ ID NO: 57) polypeptide alone or in combination with LDHA after incubation with FOLRα-expressing IGROV-1 cells and an anti-FOLRα antibody for approximately 48 hours to measure IL-2 production (FIG. 6A) or 8 days to measure proliferation by live T cell counts (FIG. 6B).

FIG. 7 is a graph showing proliferation, as measured by live T cell counts, as a function of media glucose concentration of T cells expressing an ACTR (SEQ ID NO: 57) polypeptide alone or in combination with LDHA after incubation with FOLRα-expressing IGROV-1 cells and an anti-FOLRα antibody for 8 days.

FIGS. 8A-8B are graphs showing IL-2 production and proliferation of T cells expressing an ACTR (SEQ ID NO: 57) polypeptide alone or in combination with LDHA after incubation with FOLRα-expressing IGROV-1 cells and an anti-FOLRα antibody in the presence of varying concentrations of the solid tumor-relevant inhibitory molecule PGE₂ for approximately 48 hours to measure IL-2 production (FIG. 8A) or 8 days to measure proliferation by live T cell counts (FIG. 8B).

FIG. 9 is a graph showing IL-2 production of T cells expressing an ACTR (SEQ ID NO: 57) polypeptide alone or in combination with LDHA after incubation with FOLRα-expressing fixed IGROV-1 cells and an anti-FOLRα antibody in the presence of varying concentrations of the solid tumor-relevant inhibitory molecule kynurenine for approximately 48 hours to measure IL-2 production.

FIG. 10 is a graph showing proliferation as a function of antibody concentration of T cells expressing an ACTR (SEQ ID NO: 57) polypeptide alone or in combination with MCT1 after incubation with FOLRα-expressing fixed OVCAR8 cells and an anti-FOLRα antibody for 8 days to measure proliferation by ATP content.

FIGS. 11A-11C are graphs showing IL-2 production and proliferation of T cells expressing an ACTR (SEQ ID NO: 57) polypeptide alone or in combination with MCT1 after incubation with FOLRα-expressing fixed IGROV-1 cells and an anti-FOLRα antibody in the presence of varying concentrations of the solid tumor-relevant inhibitory molecule kynurenine. IL-2 production (FIG. 11A) was measured after incubating for approximately 48 hours. On day 7 cells were divided into two groups. The first group was pulsed with BrdU for for approximately 16 hours and a BrdU uptake assay (Millipore Sigma) was performed to assess proliferation (FIG. 11B). Proliferation was measured in the second group by ATP content on day 8 (FIG. 11C).

FIGS. 12A-12C are graphs depicting proliferation of T cells expressing an ACTR (SEQ ID NO: 57) polypeptide alone or in combination with MCT2 after incubation with FOLRα-expressing fixed OVCAR8 cells and an anti-FOLRα antibody in the presence of varying concentrations of the solid tumor-relevant inhibitory molecules PGE₂ (FIG. 12A), TGF-β (FIG. 12B), and kynurenine (FIG. 12C) for 8 days to measure proliferation by ATP content.

FIGS. 13A-13C are graphs showing IL-2 production and proliferation of T cells expressing an ACTR (SEQ ID NO: 57) polypeptide alone or in combination with MCT2 after incubation with FOLRα-expressing fixed IGROV-1 cells and an anti-FOLRα antibody in the presence of varying concentrations of the solid tumor-relevant inhibitory molecule kynurenine. IL-2 production (FIG. 13A) was measured after incubating for approximately 48 hours. On day 6 cells were divided into two groups. The first group was pulsed with BrdU for approximately 16 hours and a BrdU uptake assay (Millipore Sigma) was performed to assess proliferation (FIG. 13B). Proliferation was measured in the second group by ATP content on day 7 (FIG. 13C).

FIGS. 14A-14B are graphs showing IL-2 production of T cells expressing an ACTR (SEQ ID NO: 57) polypeptide alone or in combination with MCT2 after incubation with FOLRα-expressing live (FIG. 14A) or fixed (FIG. 14B) IGROV-1 cells and an anti-FOLRα antibody in the presence of varying concentrations of the solid tumor-relevant inhibitory molecule adenosine.

FIG. 15 is a graph showing proliferation as a function of antibody concentration of T cells expressing an ACTR (SEQ ID NO: 57) polypeptide alone or in combination with MCT4 after incubation with FOLRα-expressing fixed OVCAR8 cells and an anti-FOLRα antibody for 8 days.

FIG. 16 is a graph showing IL-2 production of T cells expressing an ACTR (SEQ ID NO: 57) polypeptide alone or in combination with MCT4 after incubation with FOLRα-expressing fixed IGROV-1 cells and an anti-FOLRα antibody in the presence of varying concentrations of the solid tumor-relevant inhibitory molecule PGE₂.

FIG. 17 is a graph depicting proliferation of T cells expressing an ACTR (SEQ ID NO: 57) polypeptide alone or in combination with MCT4 after incubation with FOLRα-expressing fixed OVCAR8 cells and an anti-FOLRα antibody in the presence of varying concentrations of the solid tumor-relevant inhibitory molecule TGF-β for 8 days to measure proliferation by ATP content.

FIGS. 18A-18B are graphs showing IL-2 production and proliferation of T cells expressing an ACTR (SEQ ID NO: 57) polypeptide alone or in combination with MCT4 after incubation with FOLRα-expressing fixed IGROV-1 cells and an anti-FOLRα antibody in the presence of varying concentrations of the solid tumor-relevant inhibitory molecule kynurenine. IL-2 production (FIG. 18A) was measured after incubating for approximately 48 hours. On day 6 cells were pulsed with BrdU for approximately 16 hours and a BrdU uptake assay (Millipore Sigma) was performed to assess proliferation (FIG. 18B).

### DETAILED DESCRIPTION OF DISCLOSURE

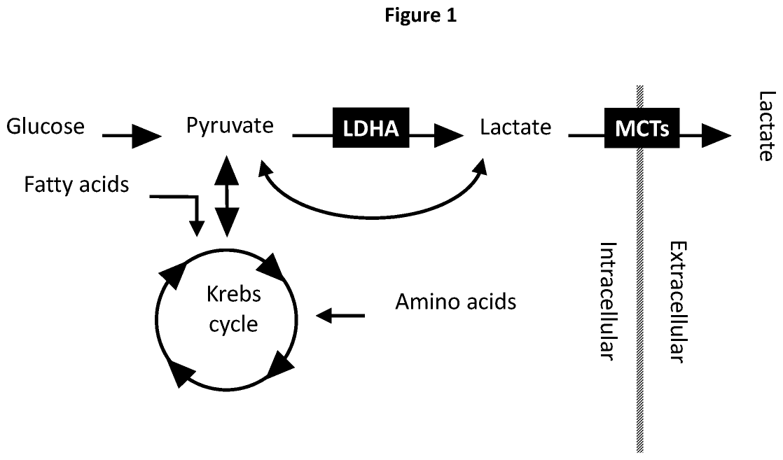

Tumor microenvironments have specific characteristics, such as low glucose, low amino acid, low pH, and/or hypoxic conditions, some of which may constrain the activity of effector immune cells such as effector T cells. The present disclosure is based, at least in part, on the development of strategies for enhancing effector immune cell activities in tumor microenvironments. In particular, the present disclosure features methods for enhancing the metabolic activity of the effector immune cells via modulating intracellular lactate concentrations therein, thereby enhancing their growth and bioactivity. Intracellular lactate concentrations can be modulated in various ways, including increasing the cellular transport of lactate (e.g., through expression or overexpression of a lactate transporter and/or through regulation of the cellular trafficking or activity of such proteins), increasing the synthesis of lactate (e.g., through expression or overexpression of an enzyme involved in lactate synthesis and/or through regulation of the cellular trafficking or activity of such proteins), and/or inhibiting a pathway that competes for substrates in the lactate synthesis pathway (e.g., through expression or overexpression of a polypeptide that inhibits a pathway that competes for lactate-synthesis substrates and/or through regulation of the cellular trafficking or activity of a protein involved in such a pathway). The present disclosure provides various approaches to modulate intracellular lactate concentrations in immune cells. Some examples are illustrated in FIG. 1, including: overexpressing an endogenous enzyme that stimulates the interconversion of lactate and pyruvate (e.g., LDHA) and/or overexpressing a lactate transporter (e.g., MCT1, MCT2, or MCT4).

The studies disclosed herein demonstrate, unexpectedly, that co-expression of a lactate-modulating polypeptide (e.g., LDHA, MCT, or PDK1) and a chimeric receptor polypeptide such as a CAR (e.g., having a 4-1BB co-stimulatory domain) or an ACTR (e.g., having a 4-1BB or CD28 co-stimulatory domain) in immune cells such as T cells exhibited superior features both in vitro and in vivo as relative to immune cells expressing only the CAR or the ACTR. For example, co-expression of LDHA, MCT1, MCT2, or MCT4 with CAR or ACTR enhanced T cell proliferation/expansion and T cell function, particularly under solid tumor microenvironment conditions (e.g., hypoxia, low glucose condition, and presence of TME inhibitors). For example, co-expression of a lactate-modulating polypeptide (e.g., LDHA, MCT, or PDK1) and a chimeric receptor polypeptide (e.g., a CAR or an ACTR) may reduce tumor growth and/or tumor formation. For example, coexpression of LDHA and ACTR enhanced T cell activity under tumor microenvironment-like conditions (e.g., low glucose, PGE₂, kynurenine). Further, coexpression of MCT1, MCT4, and MCT4 with ACTR or CAR showed enhanced T cell activity under tumor microenvironment-like conditions (e.g., low glucose, PGE₂, kynurenine, TGFβ, or adenosine).

Accordingly, the present disclosure provides modified (e.g., genetically engineered) hematopoietic cells (e.g., HSCs or immune cells) that an enhanced metabolic activity relative to native immune cells of the same type. Modulation of intracellular lactate concentrations can be achieved by any suitable approach. In some embodiments, such modified immune cells may express one or more lactate-modulating factors, for to example, lactate-modulating polypeptides. In some instances, the lactate-modulating factor may be a molecule that is directly involved in lactate synthesis, metabolism, and/or transportation, e.g., an enzyme or transporter involved in such a processes. In other instances, the lactate modulating factor may be a molecule that indirectly regulates lactate synthesis, metabolism, and/or transportation, for example, regulates expression, activity, and/or degradation of the polypeptides involved in lactate synthesis, metabolism, and/or transportation.

Such a genetically engineered immune cell may further express a chimeric receptor polypeptide, e.g., an antibody-coupled T cell receptor (ACTR) polypeptide or a chimeric antigen receptor (CAR) polypeptide. Also provided herein are uses of the genetically engineered immune cells, optionally in combination with an Fc-containing agent when needed (e.g., when the immune cells express an ACTR polypeptide), for improving immune cell proliferation, and/or an inhibiting or decreasing in target cells (e.g., target cancer cells) in a subject (e.g., a human cancer patient), e.g., via ADCC. The present disclosure also provides pharmaceutical compositions and kits comprising the described genetically engineered immune cells.

The genetically engineered immune cells described herein, expressing (e.g., over-expressing) a lactate-modulating factor, may confer at least the following advantages. The expression of the lactate-modulating factor (e.g., polypeptide or nucleic acid) would enhance the metabolic activity of a T cell. As such, the genetically engineered immune cells may proliferate better, produce more cytokines, exhibit greater anti-tumor cytotoxicity, exhibit less immunosuppressive metabolites, and/or exhibit greater T cell survival in a tumor environment (e.g., low-glucose, low amino acid, low pH, and/or hypoxic environment relative to immune cells that do not express (or do not over-express) the lactate-modulating factor (e.g., polypeptide or nucleic acid), leading to enhanced cytokine production, survival rate, cytotoxicity, and/or anti-tumor activity.

### I. Lactate-Modulating Factors

As used herein, a lactate-modulating factor can be a molecule of any type that either is involved in lactate synthesis and/or metabolism (e.g., an enzyme involved in lactate synthesis and/or metabolism, or an enzyme that inhibits a pathway that competes for substrates used in lactate synthesis), or involved in lactate cellular transportation (e.g., a cell surface lactate transporter).

In some instances, a lactate-modulating factor can be a lactate-modulating polypeptide, which refers to a polypeptide that regulates a cell's intracellular concentration of lactate. Such a lactate-modulating polypeptide may regulate intracellular lactate concentrations via any mechanism.

In some embodiments, and as exemplified in FIG. 1, a lactate-modulating polypeptide comprises a lactate transporter (i.e., a cell membrane protein that facilitates the transport of lactate across the cell membrane) and/or a regulator of the cellular trafficking or activity of such a protein. In some embodiments, a lactate-modulating polypeptide may comprise a bidirectional lactate transporter (e.g., MCT1, MCT2, or MCT4, or a functional variant thereof). In some embodiments, the lactate-modulating polypeptide comprises a genetically engineered lactate transporter, wherein the lactate transporter is mutated from a native wild-type form to mimic an activated lactate-modulating polypeptide (e.g., a phosphorylation mimic) and/or to impact its intracellular trafficking (e.g., traffic to the cell surface) such that lactate-modulating polypeptide activity is increased.

In other embodiments, as also exemplified in FIG. 1, a lactate-modulating polypeptide may comprise an enzyme involved in the synthesis of lactate (e.g., an enzyme that stimulates lactate synthesis or the conversion of lactate into another molecule). Such an enzyme may convert lactate into pyruvate. For example, a lactate-modulating polypeptide may comprise LDHA, or a functional variant thereof. In some embodiments, the lactate-modulating polypeptide may comprise a genetically engineered enzyme involved in the synthesis of lactate, wherein the enzyme is mutated from a native wild-type form to mimic an activated enzyme (e.g., a phosphorylation mimic) and/or to impact its intracellular trafficking such that lactate synthesis or conversion is increased.

In other embodiments, a lactate-modulating polypeptide may be a polypeptide that inhibits a pathway that competes for lactate-synthesis substrates and/or a regulator of the cellular trafficking or activity of a protein involved in such a pathway. For example, a lactate-modulating polypeptide may comprise PDK1, or a functional variant thereof. In some embodiments, the lactate-modulating polypeptide comprises a genetically engineered protein inhibitor, wherein the protein inhibitor is mutated from a native wild-type form to mimic an activated protein inhibitor (e.g., a phosphorylation mimic) and/or to impact its intracellular trafficking such that inhibition of the competing pathway is increased.

Any such modulating polypeptide, which may be of any suitable species (e.g., mammalian such as human), may be contemplated for use with the compositions and methods described herein.

Exemplary lactate-modulating polypeptides may include, but are not limited to, L-lactate dehydrogenase A chain (LDHA), Monocarboxylate transporter 1 (MCT1), Monocarboxylate transporter 2 (MCT2), Monocarboxylate transporter 4 (MCT4), and Pyruvate dehydrogenase kinase 1 (PDK1).

LDHA is a dehydrogenase enzyme that catalyzes the interconversion of pyruvate, a key molecule in the Krebs cycle, and lactate. The over-expression of LDHA may facilitate the conversion of lactate into pyruvate as a cell's store of pyruvate is diminished at times of high metabolic activity. This leads to an increase in the intracellular concentration of pyruvate and a decrease in the intracellular concentration of lactate and has the effect of providing flux into the Krebs cycle and increasing the transport of lactate. Accordingly, elevated expression or activity of LDHA increases the transport of lactate, leading to an ultimate elevated intracellular lactate concentration. The amino acid sequence of an exemplary human LDHA enzyme is provided below:

LDHA
(SEQ ID NO: 81)
MATLKDQLIYNLLKEEQTPQNKITVVGVGAVGMACAISILMKDLADELAL
VDVIEDKLKGEMMDLQHGSLFLRTPKIVSGKDYNVTANSKLVIITAGARQ
QEGESRLNLVQRNVNIFKFIIPNVVKYSPNCKLLIVSNPVDILTYVAWKI
SGFPKNRVIGSGCNLDSARFRYLMGERLGVHPLSCHGWVLGEHGDSSVPV
WSGMNVAGVSLKTLHPDLGTDKDKEQWKEVHKQVVESAYEVIKLKGYTSW
AIGLSVADLAESIMKNLRRVHPVSTMIKGLYGIKDDVFLSVPCILGQNGI
SDLVKVTLTSEEEARLKKSADTLWGIQKELQF

MCT proteins (e.g., MCT1, MCT2, or MCT4) are a family of monocarboxylate transporters that catalyze the bidirectional transport of lactate as well as pyruvate, ketone bodies, and other structurally-related metabolites. MCT2 has a higher affinity for lactate than MCT1 while MCT4 has a lower affinity for pyruvate than MCT1. Increased MCT expression or activity causes an increase in lactate export which then leads to an increase in glycolysis. Similarly, increased MCT expression or activity may cause an increase in the metabolic flux of lactate into biological pathways. The amino acid sequences of exemplary human MCT1, MCT2, and MCT4 proteins are provided below:

MCT1
(SEQ ID NO: 82)
MPPAVGGPVGYTPPDGGWGWAVVIGAFISIGFSYAFPKSITVFFKEIEGI
FHATTSEVSWISSIMLAVMYGGGPISSILVNKYGSRIVMIVGGCLSGCGL
IAASFCNTVQQLYVCIGVIGGLGLAFNLNPALTMIGKYFYKRRPLANGLA
MAGSPVFLCTLAPLNQVFFGIFGWRGSFLILGGLLLNCCVAGALMRPIGP
KPTKAGKDKSKASLEKAGKSGVKKDLHDANTDLIGRHPKQEKRSVFQTIN
QFLDLTLFTHRGFLLYLSGNVIMFFGLFAPLVFLSSYGKSQHYSSEKSAF
LLSILAFVDMVARPSMGLVANTKPIRPRIQYFFAASVVANGVCHMLAPLS
TTYVGFCVYAGFFGFAFGWLSSVLFETLMDLVGPQRFSSAVGLVTIVECC
PVLLGPPLLGRLNDMYGDYKYTYWACGVVLIISGIYLFIGMGINYRLLAK
EQKANEQKKESKEEETSIDVAGKPNEVTKAAESPDQKDTDGGPKEEESPV
MCT2
(SEQ ID NO: 83)
MPPMPSAPPVHPPPDGGWGWIVVGAAFISIGFSYAFPKAVTVFFKEIQQI
FHTTYSEIAWISSIMLAVMYAGGPVSSVLVNKYGSRPVVIAGGLLCCLGM
VLASFSSSVVQLYLTMGFITGLGLAFNLQPALTIIGKYFYRKRPMANGLA
MAGSPVFLSSLAPFNQYLFNTFGWKGSFLILGSLLLNACVAGSLMRPLGP
NQTTSKSKNKTGKTEDDSSPKKIKTKKSTWEKVNKYLDFSLFKHRGFLIY
LSGNVIMFLGFFAPIIFLAPYAKDQGIDEYSAAFLLSVMAFVDMFARPSV
GLIANSKYIRPRIQYFFSFAIMFNGVCHLLCPLAQDYTSLVLYAVFFGLG
FGSVSSVLFETLMDLVGAPRFSSAVGLVTIVECGPVLLGPPLAGKLVDLT
GEYKYMYMSCGAIVVAASVWLLIGNAINYRLLAKERKEENARQKTRESEP
LSKSKHSEDVNVKVSNAQSVTSERETNI
MCT4
(SEQ ID NO: 84)
MGGAVVDEGPTGVKAPDGGWGWAVLFGCFVITGFSYAFPKAVSVFFKELI
QEFGIGYSDTAWISSILLAMLYGTGPLCSVCVNRFGCRPVMLVGGLFASL
GMVAASFCRSIIQVYLTTGVITGLGLALNFQPSLIMLNRYFSKRRPMANG
LAAAGSPVFLCALSPLGQLLQDRYGWRGGFLILGGLLLNCCVCAALMRPL
VVTAQPGSGPPRPSRRLLDLSVFRDRGFVLYAVAASVMVLGLFVPPVFVV
SYAKDLGVPDTKAAFLLTILGFIDIFARPAAGFVAGLGKVRPYSVYLFSF
SMFFNGLADLAGSTAGDYGGLVVFCIFFGISYGMVGALQFEVLMAIVGTH
KFSSAIGLVLLMEAVAVLVGPPSGGKLLDATHVYMYVFILAGAEVLTSSL
ILLLGNFFCIRKKPKEPQPEVAAAEEEKLHKPPADSGVDLREVEHFLKAE
PEKNGEVVHTPETSV

PDK1 is a kinase which acts to inhibit pyruvate dehydrogenase (such as PDHA1), a component of the pyruvate dehydrogenase complex, via phosphorylation. The pyruvate dehydrogenase complex converts pyruvate into acetyl-CoA through decarboxylation. Increased PDK1 expression or activity—and subsequent inhibition of pyruvate dehydrogenase—increases the amount of pyruvate available for LDHA-mediated conversion to lactate. The amino acid sequence of an exemplary human PDK1 enzyme is provided below:

PDK1
(SEQ ID NO: 85)
MRLARLLRGAALAGPGPGLRAAGFSRSFSSDSGSSPASERGVPGQVDFYA
RFSPSPLSMKQFLDFGSVNACEKTSFMFLRQELPVRLANIMKEISLLPDN
LLRTPSVQLVQSWYIQSLQELLDFKDKSAEDAKAIYERPRRTWLQVSSLC
CMACKMIFTDTVIRIRNRHNDVIPTMAQGVIEYKESFGVDPVTSQNVQYF
LDRFYMSRISIRMLLNQHSLLFGGKGKGSPSHRKHIGSINPNCNVLEVIK
DGYENARRLCDLYYINSPELELEELNAKSPGQPIQVVYVPSHLYHMVFEL
FKNAMRATMEHHANRGVYPPIQVHVTLGNEDLTVKMSDRGGGVPLRKIDR
LFNYMYSTAPRPRVETSRAVPLAGFGYGLPISRLYAQYFQGDLKLYSLEG
YGTDAVIYIKALSTDSIERLPVYNKAAWKHYNTNHEADDWCVPSREPKDM
TTFRSA

The lactate-modulating polypeptide may be a naturally-occurring polypeptide from a suitable species, for example, a mammalian lactate-modulating polypeptide such as those derived from human or a non-human primate. Such naturally-occurring polypeptides are known in the art and can be obtained, for example, using any of the above-noted amino acid sequences as a query to search a publicly available gene database, for example GenBank. The lactate-modulating polypeptide for use in the instant disclosure may share a sequence identity of at least 85% (e.g., 90%, 95%, 97%, 98%, 99%, or above) as any of the exemplary proteins noted above.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Alternatively, the lactate-modulating polypeptide may be a functional variant of a native counterpart. Such a functional variant may contain one or more mutations outside the functional domain(s) of the native counterpart. Functional domains of a native lactate-modulating polypeptide may be known in the art or can be predicted based on its amino acid sequence. Mutations outside the functional domain(s) would not be expected to substantially affect the biological activity of the protein. In some instances, the functional variant may exhibit an increased activity in lactate transport as relative to the native counterpart. Alternatively, the functional variant may exhibit a decreased activity in lactate transport as relative to the native counterpart. Additionally, the functional variant may have increased trafficking to the cell surface. Alternatively, the functional variant may have decreased trafficking to the cell surface.

Alternatively or in addition, the functional variant may contain a conservative mutation(s) at one or more positions in the native counterpart (e.g., up to 20 positions, up to 15 positions, up to 10 positions, up to 5, 4, 3, 2, 1 position(s)). As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the lactate-modulating factor may be a molecule that regulates expression of an endogenous lactate-modulating polypeptide. Such a lactate-modulating factor may be a transcription factor or a microRNA. In some instances, the lactate-modulating factor can be a nucleic acid (e.g., microRNA, interfering RNA such as siRNA or shRNA, or antisense nucleic acid) that regulates expression of one or more enzymes involved in lactate synthesis and/or metabolism, and one or more lactate transporters. In further embodiments, the lactate-modulating factor may be a transcriptional factor that regulates expressing of one or more enzymes or transporters involved in lactate synthesis, metabolism, and/or transportation. In other embodiments, the lactate-modulating factor may be a molecule that mediates degradation of an endogenous lactate-modulating polypeptide such as those disclosed herein, for example an E3 ligase that is part of the ubiquitin/proteasome pathway. Additionally, the trafficking of an endogenous lactate-modulating polypeptide may be modulated, for example, by expressing a polypeptide that increases its trafficking to the cell surface.

### II. Chimeric Receptor Polypeptides

As used herein, a chimeric receptor polypeptide refers to a non-naturally occurring molecule that can be expressed on the surface of a host cell. A chimeric receptor polypeptide comprises an extracellular target binding domain that can target an antigen of interest (e.g., an antigen associated with a disease such as cancer or an antigen associated with a pathogen; see discussions herein). An extracellular target binding domain may bind to an antigen of interest directly (e.g., an extracellular antigen binding domain in a CAR polypeptide as disclosed herein). Alternatively, an extracellular target binding domain may bind to the antigen of interest via an intermediate, for example, an Fc-containing agent such as an antibody. A chimeric receptor polypeptide may further comprise a transmembrane domain, a hinge domain, a cytoplasmic signaling domain, one or more co-stimulatory domains, a cytoplasmic signaling domain, or a combination thereof. In some instances, the chimeric receptor polypeptide may be free of co-stimulatory domains. The chimeric receptor polypeptides are configured such that, when expressed on a host cell, the extracellular target binding domain is located extracellularly for binding to a target antigen, directly or indirectly. The optional co-stimulatory signaling domain may be located in the cytoplasm for triggering activation and/or effector signaling.

In some embodiments, chimeric receptor polypeptides described herein may further comprise a hinge domain, which may be located at the C-terminus of the extracellular target binding domain and the N-terminus of the transmembrane domain. The hinge may be of any suitable length. In other embodiments, the chimeric receptor polypeptide described herein may have no hinge domain at all. In yet other embodiments, the chimeric receptor polypeptide described herein may have a shortened hinge domain (e.g., including up to 25 amino acid residues).

In some embodiments, a chimeric receptor polypeptide as described herein may comprise, from N-terminus to C-terminus, the extracellular target binding domain, the transmembrane domain, and the cytoplasmic signaling domain. In some embodiments, a chimeric receptor polypeptide as described herein comprises, from N-terminus to C-terminus, the extracellular target binding domain, the transmembrane domain, at least one co-stimulatory signaling domain, and the cytoplasmic signaling domain. In other embodiments, a chimeric receptor polypeptide as described herein comprises, from N-terminus to C-terminus, the extracellular target binding domain, the transmembrane domain, the cytoplasmic signaling domains, and at least one co-stimulatory signaling domain.

In some embodiments, the chimeric receptor polypeptide can be an antibody-coupled T cell receptor (ACTR) polypeptide. As used herein, an ACTR polypeptide (a.k.a., an ACTR construct) refers to a non-naturally occurring molecule that can be expressed on the surface of a host cell and comprises an extracellular domain with binding affinity and specificity for the Fc portion of an immunoglobulin (“Fc binder” or “Fc binding domain”), a transmembrane domain, and a cytoplasmic signaling domain. In some embodiments, the ACTR polypeptides described herein may further include at least one co-stimulatory signaling domain.

In other embodiments, the chimeric receptor polypeptide disclosed herein may be a chimeric antigen receptor (CAR) polypeptide. As used herein, a CAR polypeptide (a.k.a., a CAR construct) refers to a non-naturally occurring molecule that can be expressed on the surface of a host cell and comprises an extracellular antigen binding domain, a transmembrane domain, and a cytoplasmic signaling domain. The CAR polypeptides described herein may further include at least one co-stimulatory signaling domain.

The extracellular antigen binding domain may be any peptide or polypeptide that specifically binds to a target antigen, including naturally occurring antigens that are associated with a medical condition (e.g., a disease), or an antigenic moiety conjugated to a therapeutic agent that targets a disease-associated antigen.

In some embodiments, the CAR polypeptides described herein may further include at least one co-stimulatory signaling domain. The CAR polypeptides are configured such that, when expressed on a host cell, the extracellular antigen-binding domain is located extracellularly for binding to a target molecule and the cytoplasmic signaling domain. The optional co-stimulatory signaling domain may be located in the cytoplasm for triggering activation and/or effector signaling.

As used herein, the phrase “a protein X transmembrane domain” (e.g., a CD8 transmembrane domain) refers to any portion of a given protein, i.e., transmembrane-spanning protein X, that is thermodynamically stable in a membrane.

As used herein, the phrase “a protein X cytoplasmic signaling domain,” for example, a CD3ζ cytoplasmic signaling domain, refers to any portion of a protein (protein X) that interacts with the interior of a cell or organelle and is capable of relaying a primary signal as known in the art, which lead to immune cell proliferation and/or activation. The cytoplasmic signaling domain as described herein differs from a co-stimulatory signaling domain, which relays a secondary signal for fully activating immune cells.

As used herein, the phrase “a protein X co-stimulatory signaling domain,” e.g., a CD28 co-stimulatory signaling domain, refers to the portion of a given co-stimulatory protein (protein X, such as CD28, 4-1BB, OX40, CD27, or ICOS) that can transduce co-stimulatory signals (secondary signals) into immune cells (such as T cells), leading to fully activation of the immune cells.

A. Extracellular Target Binding Domain

The chimeric receptor polypeptides disclosed herein comprise an extracellular domain that targets an antigen of interest (e.g., those described herein) via either direct binding or indirectly binding (through an intermediate such as an antibody). The chimeric receptor polypeptides may be ACTR polypeptides that comprise an Fc binding domain. Alternatively, the chimeric receptor polypeptides may be CAR polypeptides that comprise an extracellular antigen binding domain.

Fc Binding Domains

The ACTR polypeptides described herein comprise an extracellular domain that is an Fc binding domain, i.e., capable of binding to the Fc portion of an immunoglobulin (e.g., IgG, IgA, IgM, or IgE) of a suitable mammal (e.g., human, mouse, rat, goat, sheep, or monkey). Suitable Fc binding domains may be derived from naturally occurring proteins such as mammalian Fc receptors or certain bacterial proteins (e.g., protein A, protein G). Additionally, Fc binding domains may be synthetic polypeptides engineered specifically to bind the Fc portion of any of the antibodies described herein with high affinity and specificity. For example, such an Fc binding domain can be an antibody or an antigen-binding fragment thereof that specifically binds the Fc portion of an immunoglobulin. Examples include, but are not limited to, a single-chain variable fragment (scFv), a domain antibody, or single domain antibodies (e.g., nanobodies). Alternatively, an Fc binding domain can be a synthetic peptide that specifically binds the Fc portion, such as a Kunitz domain, a small modular immunopharmaceutical (SMIP), an adnectin, an avimer, an affibody, a DARPin, or an anticalin, which may be identified by screening a peptide combinatory library for binding activities to Fc.

In some embodiments, the Fc binding domain is an extracellular ligand-binding domain of a mammalian Fc receptor. As used herein, an “Fc receptor” is a cell surface bound receptor that is expressed on the surface of many immune cells (including B cells, dendritic cells, natural killer (NK) cells, macrophage, neutrophils, mast cells, and eosinophils) and exhibits binding specificity to the Fc domain of an antibody. Fc receptors are typically comprised of at least two immunoglobulin (Ig)-like domains with binding specificity to an Fc (fragment crystallizable) portion of an antibody. In some instances, binding of an Fc receptor to an Fc portion of the antibody may trigger antibody dependent cell-mediated cytotoxicity (ADCC) effects. The Fc receptor used for constructing an ACTR polypeptide as described herein may be a naturally-occurring polymorphism variant (e.g., the CD16 V158 variant), which may have increased or decreased affinity to Fc as compared to a wild-type counterpart. Alternatively, the Fc receptor may be a functional variant of a wild-type counterpart, which carry one or more mutations (e.g., up to 10 amino acid residue substitutions including 1, 2, 3, 4, 5, 6, 7, 8, 9, or 10 mutations) that alter the binding affinity to the Fc portion of an Ig molecule. In some instances, the mutation may alter the glycosylation pattern of the Fc receptor and thus the binding affinity to Fc.

The table below lists a number of exemplary polymorphisms in Fc receptor extracellular domains (see, e.g., Kim et al., J. Mol. Evol. 53:1-9, 2001) which may be used in any of the methods or constructs described herein:

TABLE 1
Exemplary Polymorphisms in Fc Receptors

| Amino | | | | | | | | | | |
|---|---|---|---|---|---|---|---|---|---|---|
| Acid | | | | | | | | | | |
| Number | 19 | 48 | 65 | 89 | 105 | 130 | 134 | 141 | 142 | 158 |
| FCR10 | R | S | D | I | D | G | F | Y | T | V |
| P08637 | R | S | D | I | D | G | F | Y | I | F |
| S76824 | R | S | D | I | D | G | F | Y | I | V |
| J04162 | R | N | D | V | D | D | F | H | I | V |
| M31936 | S | S | N | I | D | D | F | H | I | V |
| M24854 | S | S | N | I | E | D | S | H | I | V |
| X07934 | R | S | N | I | D | D | F | H | I | V |
| X14356 | N | N | N | S | E | S | S | S | I | I |
| (FcγRII) | | | | | | | | | | |
| M31932 | S | T | N | R | E | A | F | T | I | G |
| (FcγRI) | | | | | | | | | | |
| X06948 | R | S | E | S | Q | S | E | S | I | V |
| (FcαεI) | | | | | | | | | | |

Fc receptors are classified based on the isotype of the antibody to which it is able to bind. For example, Fc-gamma receptors (FcγR) generally bind to IgG antibodies, such as one or more subtype thereof (i.e., IgG1, IgG2, IgG3, IgG4); Fc-alpha receptors (FcαR) generally bind to IgA antibodies; and Fc-epsilon receptors (FcεR) generally bind to IgE antibodies. In some embodiments, the Fc receptor is an Fc-gamma receptor, an Fc-alpha receptor, or an Fc-epsilon receptor. Examples of Fc-gamma receptors include, without limitation, CD64A, CD64B, CD64C, CD32A, CD32B, CD16A, and CD16B. An example of an Fc-alpha receptor is FcαR1/CD89. Examples of Fc-epsilon receptors include, without limitation, FcεRI and FcεRII/CD23. The table below lists exemplary Fc receptors for use in constructing the ACTR polypeptides described herein and their binding activity to corresponding Fc domains:

TABLE 2
Exemplary Fc Receptors

| | Principal antibody | |
|---|---|---|
| Receptor name | ligand | Affinity for ligand |
| FcγRI (CD64) | IgG1 and IgG3 | High (Kd ~ 10⁻⁹M) |
| FcγRIIA (CD32) | IgG | Low (Kd > 10⁻⁷M) |
| FcγRIIB1 (CD32) | IgG | Low (Kd > 10⁻⁷M) |
| FcγRIIB2 (CD32) | IgG | Low (Kd > 10⁻⁷M) |
| FcγRIIIA (CD16a) | IgG | Low (Kd > 10⁻⁶M) |
| FcγRIIIB (CD16b) | IgG | Low (Kd > 10⁻⁶M) |
| FcεRI | IgE | High (Kd ~ 10⁻¹⁰M) |
| FcεRII (CD23) | IgE | Low (Kd > 10⁻⁷M) |
| FcαRI (CD89) | IgA | Low (Kd > 10⁻⁶M) |
| Fcα/μR | IgA and IgM | High for IgM, |
| | | Mid for IgA |
| FcRn | IgG | |

Selection of the ligand binding domain of an Fc receptor for use in the ACTR polypeptides described herein will be apparent to one of skill in the art. For example, it may depend on factors such as the isotype of the antibody to which binding of the Fc receptor is desired and the desired affinity of the binding interaction.

The extracellular antigen binding domain of any of the CAR polypeptidesln some examples, the Fc binding domain is the extracellular ligand-binding domain of CD16, which may incorporate a naturally occurring polymorphism that may modulate affinity for Fc. In some examples, the Fc binding domain is the extracellular ligand-binding domain of CD16 incorporating a polymorphism at position 158 (e.g., valine or phenylalanine). In some embodiments, the Fc binding domain is produced under conditions that alter its glycosylation state and its affinity for Fc.

The amino acid sequences of human CD16A F158 and CD16A V158 variants are provided below with the F158 and V158 residue highlighted in bold/face and underlined (signal peptide italicized):

CD16A F158 (SEQ ID NO: 86):
MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLFGSKNVSSETVNITITQGLAVSTIS
SFFPPGYQVSFCLVMVLLFAVDTGLYFSVKTNIRSSTRDWKDHKFKWRKD
PQDK
CD16A V158 (SEQ ID NO: 87):
MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLVGSKNVSSETVNITITQGLAVSTIS
SFFPPGYQVSFCLVMVLLFAVDTGLYFSVKTNIRSSTRDWKDHKFKWRKD
PQDK

In some embodiments, the Fc binding domain is the extracellular ligand-binding domain of CD16 incorporating modifications that render the ACTR polypeptide specific for a subset of IgG antibodies. For example, mutations that increase or decrease the affinity for an IgG subtype (e.g., IgG1) may be incorporated.

Any of the Fc binding domains described herein may have a suitable binding affinity for the Fc portion of a therapeutic antibody. As used herein, “binding affinity” refers to the apparent association constant or K_{A}. The K_{A} is the reciprocal of the dissociation constant, K_{D}. The extracellular ligand-binding domain of an Fc receptor domain of the ACTR polypeptides described herein may have a binding affinity K_{d} of at least 10⁻⁵, 10⁻⁶, 10⁻⁷, 10⁻⁸, 10⁻⁹, 10⁻¹⁰ M or lower for the Fc portion of antibody. In some embodiments, the Fc binding domain has a high binding affinity for an antibody, isotype(s) of antibodies, or subtype(s) thereof, as compared to the binding affinity of the Fc binding domain to another antibody, isotype(s) of antibodies, or subtypes(s) thereof. In some embodiments, the extracellular ligand-binding domain of an Fc receptor has specificity for an antibody, isotype(s) of antibodies, or subtype(s) thereof, as compared to binding of the extracellular ligand-binding domain of an Fc receptor to another antibody, isotype(s) of antibodies, or subtypes(s) thereof.

Other Fc binding domains as known in the art may also be used in the ACTR constructs described herein including, for example, those described in WO2015058018A1 and PCT Application No.: PCT/US2018/015999, the relevant disclosures of each of which are incorporated by reference for the purpose and subject matter referenced herein.

Extracellular Antigen Binding Domains

The CAR polypeptides described herein comprise an extracellular antigen binding domain, which re-directs the specificity of immune cells expressing the CAR polypeptide. As used herein, “an extracellular antigen binding domain” refers to a peptide or polypeptide having binding specificity to a target antigen of interest, which can be a naturally occurring antigen associated with a medical condition (e.g., a disease), or an antigenic moiety conjugated to a therapeutic agent that targets a disease-associated antigen. The extracellular antigen binding domain as described herein does not comprise an extracellular domain of an Fc receptor, and may not bind to the Fc portion of an immunoglobulin. An extracellular domain that does not bind to an Fc fragment means that the binding activity between the two is not detectable using a conventional assay or only background or biologically insignificant binding activity is detected using the conventional assay.

In some instances, the extracellular antigen binding domain of any CAR polypeptides described herein is a peptide or polypeptide capable of binding to a cell surface antigen (e.g., a tumor antigen), or an antigen (or a fragment thereof) that is complex with a major histocompatibility complex and be presented on the cell surface of an antigen-presenting cell. Such an extracellular antigen binding domain may be a single-chain antibody fragment (scFv), which may be derived from an antibody that binds the target cell surface antigen with a high binding affinity. Table 3 below lists exemplary cell-surface target antigens and exemplary antibodies binding to such.

TABLE 3
Exemplary Cell Surface Target Antigen and Exemplary Antibodies Binding to Such

| Exemplary Target | | Exemplary Target | Exemplary Antibodies |
|---|---|---|---|
| Antigens | Exemplary Antibodies | Antigens | and Fc-fusion Agents |
| CD137 (4-1BB) | utomilumab | CD74 | milatuzumab |
| Trophoblast | naptumomab estafenatox | HLA-DR | IMMU-114 |
| glycoprotein (5T4) | | | |
| Adenosine A2a receptor | anti-A2aR mAbs | Hsp70 | mi-TUMEXtx |
| (A2aR) | | | |
| Alk-1 protein kinase | ascrinvacumab | Hsp 90 | ZSG-102 |
| (ACVRL1) | | | |
| ADAM-10 (ADAM10) | 8C7 | ICAM-1 | BI-505 |
| TACE (ADAM17) | MEDI-3622 | Inducible T-cell co- | GSK-3359609 |
| | | stimulator (ICOS) | |
| ADAM-28 (ADAM28) | GFC-201 | Immunoglobulin kappa | KappaMab |
| | | (Ig kappa) | |
| CD156; | MAB-1031 | Immunoglobulin | LambdaMab |
| | | antigen | |
| Immunoglobulin G1; | | (Ig lambda) | |
| Immunoglobulin G2 | | | |
| (ADAM8) | | | |
| ADAM-9 (ADAM9) | AEX-6003 | IL-6 receptor (IL-6R) | tocilizumab |
| Anterior gradient | agtuzumab | IL-7 receptor (IL-7R) | anti-IL7R mAbs |
| protein 2 homolog | | | |
| (AGR2) | | | |
| Anaplastic lymphoma | KTN-0125 | IL-13 receptor alpha 1 | ASLAN-004 |
| kinase (ALK) | | subunit (IL13RA1) | |
| Angiopoietin ligand-2 | vanucizumab | IL-13 receptor alpha 2 | anti-IL13RA2 mAbs |
| (Ang-2); Vascular | | subunit (IL13RA2) | |
| endothelial growth | | | |
| factor-A (VEGF-A) | | | |
| Lactadherin (Anti- | TriAb (11D10) | IL-1 receptor accessory | CAN-04 |
| idiotype) | | protein (IL1RAP) | |
| Tumor necrosis factor | BION-1301 | IL-2 receptor beta (IL2R | Mikbeta1 |
| ligand 13 (APRIL) | | beta) | |
| Aspartate beta- | PAN-622 | Immunoglobulin like | BAY-1905254 |
| hydroxylase (ASPH) | | domain receptor 2 | |
| | | (ILDR2) | |
| Axl tyrosine kinase | BA-3011 | Integrin alpha-X/beta-1 | anti-Integrin a10b1 mAbs |
| (AXL) | | (Integrin a10b1) | |
| CD276 antigen (B7-H3) | BVD m276; hu8H9 | Integrin alpha-3/beta-1 | BCMab-1 |
| | | (Integrin a3b1) | |
| V-set domain-containing | FPA-150 | Integrin alpha-6/beta-4 | 90Y-ITGA6B4 |
| T-cell activation | | (Integrin a6b4) | |
| inhibitor 1 (VTCN1; | | | |
| also B7-H4) | | | |
| B-cell activating factor; | blisibimod | Integrin alpha-9 | GND-001 |
| (BAFF; also TNFSF13B | | (Integrin a9) | |
| and CD257) | | | |
| B-cell activating factor | VAY736 | CD49b (Integrin alpha 2) | Vatelizumab |
| receptor; (BAFF-R; also | | | |
| TNFSF13C and CD268) | | | |
| BAG molecular | anti-BAG3 mAbs | CD49c (Integrin alpha 3) | anti-CD49c mAbs |
| chaperone regulator 3 | | | |
| (BAG3) | | | |
| Basigin (BSG; CD147) | cHAb18 | CD49d; (Integrin alpha 4) | anti-CD49d mAbs |
| B-cell maturation | SEA-BCMA | CD51 | abituzumab |
| antigen (BCMA; also | | | |
| TNFRSF17) | | | |
| ADP ribosyl cyclase-2 | OX-001 | CD29 (integrin beta 1) | OS-2966 |
| (BST1) | | | |
| B and T lymphocyte | 40E4 | CD61 (Integrin beta 3) | anti-CD61 mAbs |
| attenuator (BTLA) | | | |
| Complement C5a | neutrazumab | Jagged-1 | anti-Jagged-1 mAbs |
| receptor (C5aR) | | | |
| CACNA2D1 calcium | anti-CACNA2D1 mAbs | Kidney-associated | AB-3A4 |
| channel subunit | | antigen 1 (KAAG1) | |
| (CACNA2D1) | | | |
| Carbonic anhydrase-IX | G250 | Potassium channel | Y-4 |
| (CAIX) | | subfamily K member 9 | |
| | | (KCNK9) | |
| Calreticulin (CALR) | Anti-CALR mAbs | KIR2DL1/2L3 | lirilumab |
| Caveolin 1 (CAV1) | anti-CAV1 mAbs | tyrosine-protein kinase | CDX-0158 |
| | | kit (KIT) | |
| Carbonic anhydrase-XII | 177Lu-6A10-Fab; anti- | L1CAM | anti-L1CAM mAbs |
| (CAXII) | CAXII mAbs | | |
| CCR2 chemokine | plozalizumab | Death receptor 5 (DR5) | APOMAB |
| receptor (CCR2) | | | |
| CCR3 chemokine | anti-CCR3 mAbs | CD223 (LAG3) | relatlimab |
| receptor (CCR3) | | | |
| CCR4 chemokine | mogamulizumab | Lewis Y | hu3S193; MB311 |
| receptor (CCR4) | | | |
| CCR5 chemokine | PRO 140; | Zinc transporter | SGN-LIV1 |
| receptor (CCR5) | CCR5mAb004 | SLC39A6 (LIV1) | |
| CCR7 chemokine | anti-CCR7 mAbs | Lysyl oxidase-like | AB-0023 |
| receptor (CCR7) | | protein 2 (LOXL2) | |
| CCR9 chemokine | anti-CCR9 mAbs | Leucine rich repeat | ABBV-085 |
| receptor (CCR9) | | containing protein 15 | |
| | | (LRRC15) | |
| Interleukin-3 receptor | CSL362; KHK2823 | Leucine rich repeat- | ARGX-115 |
| alpha (IL3RA; CD123) | | containing protein 32 | |
| | | (LRRC32) | |
| Aminopeptidase N | MI-130110 | Lymphocyte antigen 75 | MEN-1309 |
| (CD13) | | (LY75) | |
| Prominin 1 (CD133) | anti-CD133 mAbs | Ly6/PLAUR domain- | BAY-1129980 |
| | | containing protein 3 | |
| | | (LYPD3) | |
| Syndecan-1 (CD138) | indatuximab ravtansine | Melanoma associated | LxC-002 |
| | | antigen (MAGE peptide | |
| | | presented in MHC) | |
| CD160 | ELB-021 | Matriptase (STU) | anti-ST14 mAbs |
| Activated leukocyte cell | CX-2009 | MICA/B | IPH4301 |
| adhesion molecule | | | |
| (CD166) | | | |
| B-lymphocyte antigen | MOR208 | MIF/HLA-A2 (MIF | RL21A |
| CD19 | | peptide presented in | |
| | | MHC) | |
| B-lymphocyte antigen | rituximab; | Anti-mullerian hormone | GM-102 |
| CD20 | obinituzumab; | II (MHR2) | |
| | ocaratuzumab | | |
| Membrane glycoprotein | samalizumab | MMP1/HLA | Anti-MMP1/HLA mAbs |
| OX2 CD200 | | (MMP1 peptide | |
| | | presented in MHC1) | |
| CD22 | epratuzumab | Metalloprotease-9 | andecaliximab |
| | | (MMP9) | |
| Immunoglobulin epsilon | lumiliximab | Mesothelin (MSLN) | MORAb-009 |
| Fc receptor II (CD23) | | | |
| Signal transducer CD24 | anti-CD24 mAbs | Mucin 1 (MUC1) | PankoMab-GEX |
| IL-2 receptor alpha | 90Y-daclizumab | Mucin 13 (MUC13) | anti-MUC13 mAbs |
| subunit CD25 | | | |
| CD27 | varilumab | Endomucin (MUC14) | anti-MUC14 mAbs |
| CD28 | theralizumab | Mucin 16 (MUC16) | sofituzumab |
| CD3 | Muromonab-CD3 | Cell surface | AA98 |
| | (OKT3) | glycoprotein MUC18 | |
| | | (CD146) | |
| CD30 | brentuximab vedotin | Mucin 5AC (MUC5 AC) | ensituximab |
| Immunoglobulin gamma | BI-1206 | N-glycolyl GM3 | 99mTc-labeled 14F7 |
| Fc receptor IIB | | (NeuGcGM3) | |
| (CD32B) | | | |
| CD33 | lintuzumab | Sodium-dependent | XMT-1536 |
| | | phosphate transport | |
| | | protein 2B (SLC34A2) | |
| CD37 | otlertuzumab | Nucleolin (NCL) | anti-nucleolin mAbs |
| ADP ribosyl cyclase-1 | daratumumab | Nectin-4 | enfortumab vedotin |
| (CD38) | | | |
| CD39 | OREG-103 | Neurofibromin (NF1) | anti-neurofibromin mAbs |
| CD4 | IT-1208 | NGcGM3 ganglioside | racotumomab |
| CD40 | lucatumumab | NKG2A | monalizumab |
| CD43 | leukotuximab | non-POU domain- | PAT-LM1 |
| | | containing octamer- | |
| | | binding protein (NONO) | |
| CD44 | RG7356 | Notch-1 | brontictuzumab |
| CD45 | 131I-BC8 | CD73 | oleclumab |
| Membrane cofactor | AugmAb | Netrin-1 (NTN1) | NP-137 |
| protein (CD46) | | | |
| CD47 | Hu5F9-G4 | OX-40 | PF-04518600 |
| CD52 | alemtuzumab | P2X purinoceptor 7 | BIL-010t |
| | | (P2RX7) | |
| CD55 | PAT-SC1 | FGF receptor (pan | MM-161 |
| | | FGFR) | |
| Neural cell adhesion | IMGN-901 | Integrin (Pan integrin) | NOD201 |
| molecule 1; (CD56) | | | |
| T-cell differentiation | itolizumab | P-cadherin, also | PCA-062 |
| antigen CD6 | | cadherin-3 (CDH3) | |
| CD70 | SGN-70 | Programmed cell death | pembrolizumab |
| | | protein 1 (PD-1) | |
| CD79b | polatuzumab vedotin | Programmed cell death | avelumab; Euchloe H12 |
| | | ligand 1 (PD-L1) | |
| CD8 | anti-CD8 mAbs | Programmed cell death | rHIgM12B7 |
| | | ligand 2 (PD-L2) | |
| CD80 | galiximab | PDGF receptor alpha | olaratumumab |
| | | (PDGFRA) | |
| CD98 | IGN-523 | Placenta specific protein | anti-PLAC1 mAbs |
| | | 1 (PLAC1) | |
| CD99 | NV-103 | PR1/HLA (PR1 peptide | anti-PR1/HLA mAbs |
| | | in MHC) | |
| Cadherin-1 (CDH1) | anti-CDH1 mAbs | Prolactin receptor PRLR | ABBV-176 |
| Cadherin-17 (CDH17) | anti-CDH17 mAbs | Phosphatidylserine | anti-phosphatidylserine |
| | | | mAbs |
| Cadherin 19 (CDH19) | anti-CDH19 mAbs | Prostate stem cell | anti-PSCA mAbs |
| | | antigen (PSCA) | |
| Cadherin-6 (CDH6) | HKT-288 | Glutamate | ATL-101 |
| | | carboxypeptidase II | |
| | | (PSMA) | |
| CD66a (CEACAM1) | CM-24 | Parathyroid hormone- | CAL |
| | | related protein (PTH-rP) | |
| CD66e (CEACAM5) | IMMU-130 | Tyrosine-protein kinase- | cofetuzumab pelidotin |
| | | like 7 (PTK7) | |
| CD66c; CD66e | NEO-201 | Protein tyrosine | PRL3-zumab |
| (CEACAM5/6) | | phosphatase IVA3 | |
| | | (PTP4A3) | |
| Claudin 18 (Claudin | IMAB362 | Poliovirus receptor | COM-701 |
| 18.2) | | related immunoglobulin | |
| | | domain containing | |
| | | (PVRIG) | |
| Claudin 6 | IMAB027 | Receptor activator of | denosumab |
| | | nuclear factor kappa- | |
| | | B ligand (RANKL) | |
| SLAM family member 7 | elotuzumab | Recepteur d'origine | anti-RON mAbs |
| (CS1) | | nantais | |
| | | (RON) | |
| colony stimulating | cabiralizumab | Tyrosine-protein kinase | cirmtuzumab |
| factor-1 receptor | | transmembrane | |
| (CSF1R) | | receptor ROR1 (ROR1); | |
| | | also NTRKR1 | |
| Cytotoxic T-lymphocyte | ipilumumab | Tyrosine-protein kinase | BA-3021 |
| protein-4 (CTLA4) | | transmembrane | |
| | | receptor ROR2 (ROR2); | |
| | | also NTRKR2 | |
| Coxsackievirus and | anti-CXADR mAbs | R-spondin-3 (RSPO3) | rosmantuzumab |
| adenovirus receptor | | | |
| (CXADR) | | | |
| CXCR2 chemokine | anti-CXCR2 mAbs | Sphingosine-1- | EDD7H9 |
| receptor | | phosphate receptor 3 | |
| | | (S1PR3) | |
| CXCR3 chemokine | anti-CXCR3 mAbs | Surface Antigen In | IGN-786 |
| receptor | | Leukemia (SAIL) | |
| CXCR4 chemokine | ulocuplumab | Semaphorin-4D | VX-15 |
| receptor | | (SEMA4D) | |
| CXCR5 chemokine | STI-B030X | carbohydrate antigen 19- | MVT-1075 |
| receptor | | 9 (CA 19-9) | |
| CXCR7 chemokine | anti-CXCR7 mAbs | Sialyl Thomsen nouveau | anti-STn mAbs |
| receptor | | antigen (STn) | |
| DCLK1 | anti-DCLK1 mAbs | Sialic acid-binding Ig- | AK-002 |
| | | like lectin 8 (Siglec-8) | |
| Dickkopf-related protein | BHQ-880 | Sialic acid-binding Ig- | anti-Siglec-9 mAbs |
| 1 (DKK1) | | like lectin 9 (Siglec-9) | |
| DLK1 | ADCT-701 | Signal Regulatory | OSE-172 |
| | | Protein Alpha (SIRPA) | |
| Delta-like protein ligand | SC16LD6.5 | CD48; also SLAM | SGN-CD48A |
| 3 (DLL3) | | family member 2 | |
| | | (SLAMF2) | |
| Delta-like protein ligand | navicixizumab | CD352; SLAM family | SGN-CD352A |
| 4 (DLL4); VEGF | | member 6 (SLAMF6) | |
| (VEGF) | | | |
| Dipeptidyl peptidase-4 | YSCMA | Neutral amino acid | KM-8094 |
| (DPP4), (also CD26) | | transporter B0 | |
| | | (SLC1A5) | |
| Death receptor-3 (DR3) | PTX-35 | Somatostatin 2 receptor | XmAb-18087 |
| | | (SSTR2) | |
| TRAIL-1 receptor | HuYON007 MultYbody | Stabilin 1 (STAB1) | FP-1305 |
| (DR4) | | | |
| TRAIL-1 receptor; | DR4/DR5 Surrobody | Metalloreductase | 89Zr-DFO-MSTP2109A |
| TRAIL-2 receptor | | (STEAP1) | |
| (DR4/DR5) | | | |
| TRAIL-2 receptor | DS-8273 | Survivin | anti-sun ivin mAbs |
| (DR5) | | | |
| EGF-like protein 6 | anti-EGFL6 mAbs | TAG-72 | 90Y-IDEC-159 |
| (EGFL6) | | | |
| Epidermal growth factor | cetuximab; Sym004; | T cell receptor (TCR) | anti-TCR mAbs |
| receptor (EGFR) | nimotuzumab | | |
| Epidermal growth factor | ABT-806 | Endosialin (TEM1) | ontuxizumab |
| receptor vIII | | | |
| (EGFRvIII) | | | |
| Epithelial membrane | ONCR-201 | Anthrax toxin receptor 1 | anti-TEM8 mAbs |
| protein 2 (EMP2) | | (ANTXR1); also TEM8 | |
| Endoglin | carotuximab | Tissue factor (TF) | MORAb-066 |
| Ectonucleotide | AGS-16C3F | Transforming growth | anti-TGFBR2 mAbs |
| pyrophosphatase/ | | factor, beta receptor II | |
| phosphodiesterase | | TGF-beta type II | |
| family member 3 | | (TGFBR2) | |
| (ENPP3) | | | |
| Prostaglandin | anti-PTGER2 mAbs | Thomsen-Friedenreich | JAA-F11 |
| E₂ receptor 2 | | Antigen | |
| (PTGER2) | | | |
| Prostaglandin | anti-PTGER4 mAbs | T cell immunoreceptor | BMS-986207 |
| E₂ receptor 4 | | with Ig and ITIM | |
| (PTGER4) | | domains (TIGIT) | |
| EpCAM | oportuzumab monatox | Hepatitis A virus cellular | CDX-014 |
| | | receptor 1 (HAVCR1); | |
| | | also TIM-1 | |
| Ephrin type-A receptor | MEDI-547 | Hepatitis A virus cellular | MBG453 |
| 2 (EphA2) | | receptor 2 (HAVCR2); | |
| | | also TIM-3 | |
| Ephrin type-A receptor | KB004 | Toll-like receptor 2 | OPN-305 |
| 3 (EphA3) | | (TLR-2) | |
| Fibroblast activation | F19 | Toll-like receptor 4 | anti-TLR4 mAbs |
| protein (FAP) | | (TLR-4) | |
| CD95 (FAS) | asunercept | Transmembrane 4 L6 | anti-TM4SF1 mAbs |
| | | family member 1 | |
| | | (TM4SF1) | |
| Fc receptor like protein | RG-6160 | Tumor necrosis factor | anti-TNFR2 mAbs |
| 5 (FCRL5) | | receptor 2 (TNFR2) | |
| FGF receptor 1 | FP-1039 | CD71 | anti-CD71 mAbs |
| (FGFR1) | | | |
| FGF receptor 2b | FPA-144 | Triggering receptor | anti-TREM1 mAbs |
| (FGFR2b) | | expressed on myeloid | |
| | | cells 1 (TREM1) | |
| FGF receptor 3 | B-701 | Tumor-associated | DS-1062 |
| (FGFR3) | | calcium signal | |
| | | transducer 2 (Trop-2) | |
| fms-like tyrosine kinase | Flysyn | TWEAK Receptor | MRT-101 |
| 3 (FLT3) | | (TWEAKR) | |
| Folate receptor alpha | farletuzumab; | Tyrosine-protein kinase | ELB-031 |
| (FOLR1) | IMGN853; KHK2805 | receptor TYRO3 | |
| | | (TYRO3) | |
| Folate receptor beta | anti-FOLR beta mAbs | Urokinase receptor | MNPR-101 |
| (FOLR2) | | (uPAR) | |
| Frizzled-1; Frizzled-2; | vantictumab | VEGF-2 (VEGFR2) | ramucirumab |
| Frizzled-5; Frizzled-7; | | | |
| Frizzled-8; | | | |
| (FZD1, 2, 5, 7, 8) | | | |
| Follistatin-like protein 1 | anti-FSTL1 mAbs | Vimentin | pritumumab |
| (FSTL1) | | | |
| Fucosyl-GM1 | BMS-986012 | V-domain Ig suppressor | JNJ-61610588 |
| | | of T cell activation | |
| | | (VISTA) | |
| Frizzled-10 (FZD10) | OTSA-101 | Integrin alpha-4/beta-l | natalizumab |
| GCSF-R (Also, CD114 | CSL324 | Immunoglobulin iota | anti-VPREB1 mAbs |
| and CSFR3) | | chain (VPREB1) | |
| Galectin 3 binding | MP-1959 | Wilms tumor protein | ESK1 |
| protein (LGALS3) | | (WT1/HLA); WT1 | |
| | | peptide presented in | |
| | | MHC | |
| Guanylate cyclase 2C | TAK-164 | Glypican-3 (GPC3) | codrituzumab |
| (GUCY2C) | | | |
| GD2 | dinutuximab | Transmembrane | CDX-011 |
| | | glycoprotein NMB | |
| | | (GPNMB) | |
| GD3 | PF-06688992 | Leucine-rich repeat- | BNC-101 |
| | | containing G-protein | |
| | | coupled receptor 5 | |
| | | (LGR5) | |
| glucocorticoid-induced | BMS-986156 | G-protein coupled | JNJ-64407564 |
| TNFR-related protein | | receptor family C group | |
| (GITR) | | 5 member D (GPRC5D) | |
| glucocorticoid-induced | EU-102 | Ferritin | Ferritarg P |
| TNFR-related protein | | | |
| ligand (GITRL) | | | |
| premelanocyte protein | anti-PMEL mAbs | Erbb2 tyrosine kinase | trastuzumab; pertuzumab; |
| (PMEL) | | (HER2) | margetuximab |
| Cell surface A33 antigen | Anti-GPA33 mAbs | Erbb3 tyrosine kinase | patritumab |
| (GPA33) | | (HER3) | |
| Glypican-1 (GPC1) | MIL-38 | Globo H | OBI-888 |

The extracellular antigen binding domain may comprise an antigen binding fragment (e.g., a scFv) derived from any of the antibodies listed in Table 3 depending upon the target antigen of interest.

In other embodiments, the extracellular antigen binding domain of any of the CAR polypeptides described herein may be specific to a pathogenic antigen, such as a bacterial antigen, a viral antigen, or a fungal antigen. Some examples are provided below: influenza virus neuraminidase, hemagglutinin, or M2 protein, human respiratory syncytial virus (RSV) F glycoprotein or G glycoprotein, herpes simplex virus glycoprotein gB, gC, gD, or gE, Chlamydia MOMP or PorB protein, Dengue virus core protein, matrix protein, or glycoprotein E, measles virus hemagglutinin, herpes simplex virus type 2 glycoprotein gB, poliovirus I VP1, envelope glycoproteins of HIV 1, hepatitis B core antigen or surface antigen, diptheria toxin, Streptococcus 24M epitope, Gonococcal pilin, pseudorabies virus g50 (gpD), pseudorabies virus II (gpB), pseudorabies virus III (gpC), pseudorabies virus glycoprotein H, pseudorabies virus glycoprotein E, transmissible gastroenteritis glycoprotein 195, transmissible gastroenteritis matrix protein, or human hepatitis C virus glycoprotein E1 or E2.

In addition, the extracellular antigen binding domain of the CAR polypeptide to described herein may be specific to a tag conjugated to a therapeutic agent, which targets an antigen associated with a disease or disorder (e.g., a tumor antigen or a pathogenic antigen as described herein). In some instances, the tag conjugated to the therapeutic agent can be antigenic and the extracellular antigen binding domain of the CAR polypeptide can be an antigen-binding fragment (e.g., scFv) of an antibody having high binding affinity and/or specificity to the antigenic tag. Exemplary antigenic tags include, but are not limited to, biotin, avidin, a fluorescent molecule (e.g., GFP, YRP, luciferase, or RFP), Myc, Flag, His (e.g., poly His such as 6× His), HA (hemeagglutinin), GST, MBP (maltose binding protein), KLH (keyhole limpet hemocyanins), trx, T7, HSV, VSV (e.g., VSV-G), Glu-Glu, V5, e-tag, S-tag, KT3, E2, Au1, Au5, and/or thioredoxin.

In other instances, the tag conjugated to the therapeutic agent is a member of a ligand-receptor pair and the extracellular antigen binding domain comprises the other member of the ligand-receptor pair or a fragment thereof that binds the tag. For example, the tag conjugated to the therapeutic agent can be biotin and the extracellular antigen binding domain of the CAR polypeptide can comprise a biotin-binding fragment of avidin. See, e.g., Urbanska et al., 2012, Lohmueller et al., 2018. Other examples include anti-Tag CAR, in which the extracellular antigen binding domain is a scFv fragment specific to a protein tag, such as FITC (Tamada et al., 2012, Kim et al., 2015; Cao et al., 2016; and Ma et al., 2016), PNE (Rodgers et al., 2016), La-SS-B (Cartellieri et al., 2016), Biotin (Lohmullular et al., 2017), and Leucine-Zipper (Cho et al., 2018). Selection of the antigen binding domain for use in the CAR polypeptides described herein will be apparent to one of skill in the art. For example, it may depend on factors such as the type of target antigen and the desired affinity of the binding interaction.

The extracellular antigen binding domain of any of the CAR polypeptides described herein may have suitable binding affinity for a target antigen (e.g., any one of the targets described herein) or antigenic epitopes thereof. As used herein, “binding affinity” refers to the apparent association constant or K_{A}. The K_{A} is the reciprocal of the dissociation constant (K_{D}). The extracellular antigen binding domain for use in the CAR polypeptides described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}. Higher affinity binding of an extracellular antigen binding domain for a first antigen relative to a second antigen can be indicated by a higher K_{A} (or a smaller numerical value K_{D}) for binding the first antigen than the K_{A} (or numerical value K_{D}) for binding the second antigen. In such cases, the extracellular antigen binding domain has specificity for the first antigen (e.g., a first protein in a first conformation or mimic thereof) relative to the second antigen (e.g., the same first protein in a second conformation or mimic thereof or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

B. Transmembrane Domain

The transmembrane domain of the chimeric receptor polypeptides (e.g., ACTR polypeptides or CAR polypeptides) described herein can be in any form known in the art. As used herein, a “transmembrane domain” refers to any protein structure that is thermodynamically stable in a cell membrane, preferably a eukaryotic cell membrane. A transmembrane domain compatible for use in the chimeric receptor polypeptides used herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times).

Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the chimeric receptor polypeptide described herein is derived from a Type I single-pass membrane protein. Single-pass membrane proteins include, but are not limited to, CD8α, CD8β, 4-1BB/CD137, CD27, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CDS, CD9, CD22, CD37, CD80, CD86, CD40, CD40L/CD154, VEGFR2, FAS, and FGFR2B. In some embodiments, the transmembrane domain is from a membrane protein selected from the following: CD8α, CD8β, 4-1BB/CD137, CD28, CD34, CD4, FcεRIγ, CD16, OX40/CD134, CD3ζ, CD3ε, CD3γ, CD3δ, TCRα, CD32, CD64, VEGFR2, FAS, and FGFR2B. In some examples, the transmembrane domain is of CD8 (e.g., the transmembrane domain is of CD8α). In some examples, the transmembrane domain is of 4-1BB/CD137. In other examples, the transmembrane domain is of CD28. In some cases, the chimeric receptor polypeptide described herein may be free of a hinge domain from any non-CD16A receptor. In some instances, such a chimeric receptor polypeptide may be free of any hinge domain. Alternatively or in addition, such a chimeric receptor polypeptide may comprise two or more co-stimulatory regions as described herein. In other examples, the transmembrane domain is of CD34. In yet other examples, the transmembrane domain is not derived from human CD8α. In some embodiments, the transmembrane domain of the chimeric receptor polypeptide is a single-pass alpha helix.

Transmembrane domains from multi-pass membrane proteins may also be compatible for use in the chimeric receptor polypeptides described herein. Multi-pass membrane proteins may comprise a complex alpha helical structure (e.g., at least 2, 3, 4, 5, 6, 7 or more alpha helices) or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side. Either one or multiple helix passes from a multi-pass membrane protein can be used for constructing the chimeric receptor polypeptide described herein.

Transmembrane domains for use in the chimeric receptor polypeptides described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of each of which are incorporated by reference herein.

In some embodiments, the amino acid sequence of the transmembrane domain does not comprise cysteine residues. In some embodiments, the amino acid sequence of the transmembrane domain comprises one cysteine residue. In some embodiments, the amino acid sequence of the transmembrane domain comprises two cysteine residues. In some embodiments, the amino acid sequence of the transmembrane domain comprises more than two cysteine residues (e.g., 3, 4, 5, or more).

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence.

The hydropathy, hydrophobic or hydrophilic characteristics of a protein or protein segment, can be assessed by any method known in the art including, for example, the Kyte and Doolittle hydropathy analysis.

C. Co-Stimulatory Signaling Domains

Many immune cells require co-stimulation, in addition to stimulation of an antigen-specific signal, to promote cell proliferation, differentiation and survival, as well as to activate effector functions of the cell. In some embodiments, the chimeric receptor polypeptides, such as ACTR or CAR polypeptides, described herein comprise at least one co-stimulatory signaling domain. In certain embodiments, the chimeric receptor polypeptides may contain a CD28 co-stimulatory signaling domain or a 4-1BB (CD137) co-stimulatory signaling domain. The term “co-stimulatory signaling domain,” as used herein, refers to at least a fragment of a co-stimulatory signaling protein that mediates signal transduction within a cell to induce an immune response such as an effector function (a secondary signal). As known in the art, activation of immune cells such as T cells often requires two signals: (1) the antigen specific signal (primary signal) triggered by the engagement of T cell receptor (TCR) and antigenic peptide/MHC complexes presented by antigen presenting cells, which typically is driven by CD3ζ as a component of the TCR complex; and (ii) a co-stimulatory signal (secondary signal) triggered by the interaction between a co-stimulatory receptor and its ligand. A co-stimulatory receptor transduces a co-stimulatory signal (secondary signal) as an addition to the TCR-triggered signaling and modulates responses mediated by immune cells, such as T cells, NK cells, macrophages, neutrophils, or eosinophils.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the chimeric receptor polypeptides described herein. The type(s) of co-stimulatory signaling domain is selected based on factors such as the type of the immune cells in which the chimeric receptor polypeptides would be expressed (e.g., T cells, NK cells, macrophages, neutrophils, or eosinophils) and the desired immune effector function (e.g. ADCC). Examples of co-stimulatory signaling domains for use in the chimeric receptor polypeptides may be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g.,4-1BB/TNFRSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNF SF 18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACl/TNFRSF13B, TL1A/TNFSF15, TNF-alpha, and TNF RIFTNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the co-stimulatory signaling domain is of 4-1BB, CD28, OX40, ICOS, CD27, GITR, HVEM, TIM1, LFA1(CD11a) or CD2, or any variant thereof.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue mutations (e.g., 1, 2, 3, 4, 5, or 8) such as amino acid substitutions, deletions, or additions as compared to a wild-type counterpart. Such co-stimulatory signaling domains comprising one or more amino acid variations (e.g., amino acid substitutions, deletions, or additions) may be referred to as variants.

Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation. For example, mutation of residues 186 and 187 of the native CD28 amino acid sequence may result in an increase in co-stimulatory activity and induction of immune responses by the co-stimulatory domain of the chimeric receptor polypeptide. In some embodiments, the mutations are substitution of a lysine at each of positions 186 and 187 with a glycine residue of the CD28 co-stimulatory domain, referred to as a CD28_{LL→GG} variant. Additional mutations that can be made in co-stimulatory signaling domains that may enhance or reduce co-stimulatory activity of the domain will be evident to one of ordinary skill in the art. In some embodiments, the co-stimulatory signaling domain is of 4-1BB, CD28, OX40, or CD28_{LL→GG} variant.

In some embodiments, the chimeric receptor polypeptides may contain a single co-stimulatory domain such as, for example, a CD27 co-stimulatory domain, a CD28 co-stimulatory domain, a 4-1BB co-stimulatory domain, an ICOS co-stimulatory domain, or an OX40 co-stimulatory domain.

In some embodiments, the chimeric receptor polypeptides may comprise more than one co-stimulatory signaling domain (e.g., 2, 3, or more). In some embodiments, the chimeric receptor polypeptide comprises two or more of the same co-stimulatory signaling domains, for example, two copies of the co-stimulatory signaling domain of CD28. In some embodiments, the chimeric receptor polypeptide comprises two or more co-stimulatory signaling domains from different co-stimulatory proteins, such as any two or more co-stimulatory proteins described herein. Selection of the type(s) of co-stimulatory signaling domains may be based on factors such as the type of host cells to be used with the chimeric receptor polypeptides (e.g., T cells or NK cells) and the desired immune effector function. In some embodiments, the chimeric receptor polypeptide comprises two co-stimulatory signaling domains, for example, two copies of the co-stimulatory signaling domain of CD28. In some embodiments, the chimeric receptor polypeptide may comprise two or more co-stimulatory signaling domains from different co-stimulatory receptors, such as any two or more co-stimulatory receptors described herein, for example, CD28 and 4-1BB, CD28 and CD27, CD28 and ICOS, CD28_{LL→GG} variant and 4-1BB, CD28 and OX40, or CD28_{LL→GG} variant and OX40. In some embodiments, the two co-stimulatory signaling domains are CD28 and 4-1BB. In some embodiments, the two co-stimulatory signaling domains are CD28_{LL→GG} variant and 4-1BB. In some embodiments, the two co-stimulatory signaling domains are CD28 and OX40. In some embodiments, the two co-stimulatory signaling domains are CD28_{LL→GG} variant and OX40. In some embodiments, the chimeric receptor polypeptides described herein may contain a combination of a CD28 and ICOSL. In some embodiments, the chimeric receptor polypeptide described herein may contain a combination of CD28 and CD27. In certain embodiments, the 4-1BB co-stimulatory domain is located N-terminal to the CD28 or CD28_{LL→GG} variant co-stimulatory signaling domain.

In some embodiments, the chimeric receptor polypeptides described herein do not comprise a co-stimulatory signaling domain.

D. Cytoplasmic Signaling Domain

Any cytoplasmic signaling domain can be used to create the chimeric receptor polypeptides described herein (e.g., ACTR polypeptides or CAR polypeptides). Such a cytoplasmic domain may be any signaling domain involved in triggering cell signaling (primary signaling) that leads to immune cell proliferation and/or activation. The cytoplasmic signaling domain as described herein is not a co-stimulatory signaling domain, which, as known in the art, relays a co-stimulatory or secondary signal for fully activating immune cells.

The cytoplasmic domain described herein may comprise an immunoreceptor tyrosine-based activation motif (ITAM) domain (e.g., at least one ITAM domain, at least two ITAM domains, or at least three ITAM domains) or may be ITAM free. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix₍₆₋₈₎YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways.

In some examples, the cytoplasmic signaling domain is of CD3ζ or FcεR1γ. In other examples, cytoplasmic signaling domain is not derived from human CD3ζ. In yet other examples, the cytoplasmic signaling domain is not derived from an Fc receptor, when the extracellular Fc-binding domain of the same chimeric receptor polypeptide is derived from CD16A.

In one specific embodiment, several signaling domains can be fused together for additive or synergistic effect. Non-limiting examples of useful additional signaling domains include part or all of one or more of TCR Zeta chain, CD28, OX40/CD134, 4-1BB/CD137, FcεRIy, ICOS/CD278, IL2R-beta/CD122, IL-2R-gamma/CD132, and CD40.

In other embodiments, the cytoplasmic signaling domain described herein is free of the ITAM motif. Examples include, but are not limited to, the cytoplasmic signaling domain of Jak/STAT, Toll-interleukin receptor (TIR), and tyrosine kinase.

E. Hinge Domain

In some embodiments, the chimeric receptor polypeptides such as ACTR polypeptides or CAR polypeptides described herein further comprise a hinge domain that is located between the extracellular ligand-binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular ligand-binding domain relative to the transmembrane domain of the chimeric receptor polypeptide can be used.

Hinge domains of any protein known in the art to comprise a hinge domain are compatible for use in the chimeric receptor polypeptides described herein. In some embodiments, the hinge domain is at least a portion of a hinge domain of a naturally occurring protein and confers flexibility to the chimeric receptor polypeptide. In some embodiments, the hinge domain is of CD8. In some embodiments, the hinge domain is a portion of the hinge domain of CD8, e.g., a fragment containing at least 15 (e.g., 20, 25, 30, 35, or 40) consecutive amino acids of the hinge domain of CD8. In some embodiments, the hinge domain is of CD28. In some embodiments, the hinge domain is a portion of the hinge domain of CD28, e.g., a fragment containing at least 15 (e.g., 20, 25, 30, 35, or 40) consecutive amino acids of the hinge domain of CD28. The hinge domain and/or the transmembrane domain may be linked to additional amino acids (e.g., 15 aa, 10-aa, 8-aa, 6-aa, or 4-aa) at the N-terminal portion, at the C-terminal portion, or both. Examples can be found, e.g., in Ying et al., Nature Medicine, 25(6):947-953 (2019).

In some embodiments, the hinge domain is of CD16A receptor, for example, the whole hinge domain of a CD16A receptor or a portion thereof, which may consists of up to 40 consecutive amino acid residues of the CD16A receptor (e.g., 20, 25, 30, 35, or 40). Such a chimeric receptor polypeptide (e.g., an ACTR polypeptide) may contain no hinge domain from a different receptor (a non-CD16A receptor).

Hinge domains of antibodies, such as an IgG, IgA, IgM, IgE, or IgD antibodies, are also compatible for use in the chimeric receptor polypeptides described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptor polypeptides described herein. In some embodiments, the hinge domain between the C-terminus of the extracellular target-binding domain and the N-terminus of the transmembrane domain is a peptide linker, such as a (GlyₓSer)ₙ linker, wherein x and n, independently can be an integer between 3 and 12, including 3, 4, 5, 6, 7, 8, 9, 10, 11, 12, or more. In some embodiments, the hinge domain is (Gly₄Ser)ₙ (SEQ ID NO:88), wherein n can be an integer between 3 and 60, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. In certain embodiments, n can be an integer greater than 60. In some embodiments, the hinge domain is (Gly₄Ser)₃ (SEQ ID NO: 89). In some embodiments, the hinge domain is (Gly₄Ser)₆ (SEQ ID NO: 90). In some embodiments, the hinge domain is (Gly₄Ser)₉ (SEQ ID NO: 91). In some embodiments, the hinge domain is (Gly₄Ser)₁₂ (SEQ ID NO: 92). In some embodiments, the hinge domain is (Gly₄Ser)₁₅ (SEQ ID NO: 93). In some embodiments, the hinge domain is (Gly₄Ser)₃₀ (SEQ ID NO: 94). In some embodiments, the hinge domain is (Gly₄Ser)₄₅ (SEQ ID NO: 95). In some embodiments, the hinge domain is (Gly₄Ser)₆₀ (SEQ ID NO: 96).

In other embodiments, the hinge domain is an extended recombinant polypeptide (XTEN), which is an unstructured polypeptide consisting of hydrophilic residues of varying lengths (e.g., 10-80 amino acid residues). Amino acid sequences of XTEN peptides will be evident to one of skill in the art and can be found, for example, in U.S. Pat. No. 8,673,860, the relevant disclosures of which are incorporated by reference herein. In some embodiments, the hinge domain is an XTEN peptide and comprises 60 amino acids. In some embodiments, the hinge domain is an XTEN peptide and comprises 30 amino acids. In some embodiments, the hinge domain is an XTEN peptide and comprises 45 amino acids. In some embodiments, the hinge domain is an XTEN peptide and comprises 15 amino acids.

Any of the hinge domains used for making the chimeric receptor polypeptide as described herein may contain up to 250 amino acid residues. In some instances, the chimeric receptor polypeptide may contain a relatively long hinge domain, for example, containing 150-250 amino acid residues (e.g., 150-180 amino acid residues, 180-200 amino acid residues, or 200-250 amino acid residues). In other instances, the chimeric receptor polypeptide may contain a medium sized hinge domain, which may contain 60-150 amino acid residues (e.g., 60-80, 80-100, 100-120, or 120-150 amino acid residues). Alternatively, the chimeric receptor polypeptide may contain a short hinge domain, which may contain less than 60 amino acid residues (e.g., 1-30 amino acids or 31-60 amino acids). In some embodiments, a chimeric receptor polypeptide (e.g., an ACTR polypeptide) described herein contains no hinge domain or no hinge domain from a non-CD16A receptor.

F. Signal Peptide

In some embodiments, the chimeric receptor polypeptide (e.g., ACTR polypeptide or CAR polypeptide) may also comprise a signal peptide (also known as a signal sequence) at the N-terminus of the polypeptide. In general, signal sequences are peptide sequences that target a polypeptide to the desired site in a cell. In some embodiments, the signal sequence targets the chimeric receptor polypeptide to the secretory pathway of the cell and will allow for integration and anchoring of the chimeric receptor polypeptide into the lipid bilayer. Signal sequences including signal sequences of naturally occurring proteins or synthetic, non-naturally occurring signal sequences that are compatible for use in the chimeric receptor polypeptides described herein will be evident to one of skill in the art. In some embodiments, the signal sequence from CD8α. In some embodiments, the signal sequence is from CD28. In other embodiments, the signal sequence is from the murine kappa chain. In yet other embodiments, the signal sequence is from CD16.

G. Examples of ACTR Polypeptides

Exemplary ACTR constructs for use with the methods and compositions described herein may be found, for example, in the instant description and figures or may be found in PCT Patent Publication No.: WO2016040441A1, WO2017/161333, and PCT Application No.: PCT/US2018/015999, each of which is incorporated by reference herein for this purpose. The ACTR polypeptides described herein may comprise a CD16A extracellular domain with binding affinity and specificity for the Fc portion of an IgG molecule, a transmembrane domain, and a CD3ζ cytoplasmic signaling domain. In some embodiments, the ACTR polypeptides may further include one or more co-stimulatory signaling domains, one of which may be a CD28 co-stimulatory signaling domain or a 4-1BB co-stimulatory signaling domain. The ACTR polypeptides are configured such that, when expressed on a host cell, the extracellular ligand-binding domain is located extracellularly for binding to a target molecule and the CD3ζ cytoplasmic signaling domain. The co-stimulatory signaling domain may be located in the cytoplasm for triggering activation and/or effector signaling.

In some embodiments, an ACTR polypeptide as described herein may comprise, from N-terminus to C-terminus, the Fc binding domain such as a CD16A extracellular domain, the transmembrane domain, the optional one or more co-stimulatory domains (e.g., a CD28 co-stimulatory domain, a 4-1BB co-stimulatory signaling domain, an OX40 co-stimulatory signaling domain, a CD27 co-stimulatory signaling domain, or an ICOS co-stimulatory signaling domain), and the CD3ζ cytoplasmic signaling domain.

Alternatively or in addition, the ACTR polypeptides described herein may contain two or more co-stimulatory signaling domains, which may link to each other or be separated by the cytoplasmic signaling domain. The extracellular Fc binder, transmembrane domain, optional co-stimulatory signaling domain(s), and cytoplasmic signaling domain in an ACTR polypeptide may be linked to each other directly, or via a peptide linker. In some embodiments, any of the ACTR polypeptides described herein may comprise a signal sequence at the N-terminus.

Table 4 provides exemplary ACTR polypeptides described herein. These exemplary constructs have, from N-terminus to C-terminus in order, the signal sequence, the Fc binding domain (e.g., an extracellular domain of an Fc receptor), the hinge domain, and the transmembrane, while the positions of the optional co-stimulatory domain and the cytoplasmic signaling domain can be switched.

TABLE 4
Exemplary Components of ACTR polypeptides.

| Exemplary | | | | | | |
|---|---|---|---|---|---|---|
| AA | | | | | | |
| Sequence | | Extracellular | Hinge | | Co- | Cytoplasmic |
| (SEQ ID | Signal | domain of Fc | domain | Transmembrane | stimulatory | Signaling |
| NO) | Sequence | receptor (a) | (e) | domain (b) | domain (d) | domain (c) |
| 1 | CD8α | CD16A-V158 | CD8α | CD8α | 4-1BB | CD3ζ |
| | | | | | (CD137) | |
| 2 | CD8α | CD16A-V158 | CD8α | 4-1BB | 4-1BB | CD3ζ |
| | | | | (CD137) | (CD137) | |
| 3 | CD8α | CD16A-V158 | CD8α | CD28 | 4-1BB | CD3ζ |
| | | | | | (CD137) | |
| 4 | CD8α | CD16A-V158 | CD8α | CD34 | 4-1BB | CD3ζ |
| | | | | | (CD137) | |
| 5 | CD8α | CD16A-V158 | CD8α | Designed | 4-1BB | CD3ζ |
| | | | | hydrophobic | (CD137) | |
| | | | | TM domain | | |
| 6 | CD8α | CD32A | CD8α | CD8α | 4-1BB | CD3ζ |
| | | | | | (CD137) | |
| 7 | CD8α | CD16A-V158 | CD8α | CD8α | CD28 | CD3ζ |
| 8 | CD8α | CD16A-V158 | CD8α | CD8α | OX40 | CD3ζ |
| | | | | | (CD134) | |
| 9 | CD8α | CD16A-V158 | CD8α | CD8α | CD28 + | CD3ζ |
| | | | | | 4-1BB | |
| 10 | CD8α | CD16A-V158 | None | CD8α | 4-1BB | CD3ζ |
| | | | | | (CD137) | |
| 11 | CD8α | CD16A-V158 | XTEN | CD8α | 4-1BB | CD3ζ |
| | | | | | (CD137) | |
| 12 | CD8α | CD16A-V158 | CD8α | CD8α | CD28 LL to | CD3ζ |
| | | | | | GG mutant | |
| 13 | CD8α | CD16A-V158 | CD8α | CD8α | CD28 LL to | CD3ζ |
| | | | | | GG mutant + | |
| | | | | | 4-1BB | |
| 14 | CD8α | CD16A-V158 | CD8α | CD4 | 4-1BB | CD3ζ |
| | | | | | (CD137) | |
| 15 | CD8α | CD16A-V158 | CD8α | CD4 | CD28 LL to | CD3ζ |
| | | | | | GG mutant + | |
| | | | | | 4-1BB | |
| 16 | CD8α | CD16A-V158 | CD8α | FcεRIγ | 4-1BB | CD3ζ |
| | | | | | (CD137) | |
| 17 | CD8α | CD16A-V158 | CD8α | Designed | 4-1BB | CD3ζ |
| | | | | hydrophobic | (CD137) | |
| | | | | TM domain, | | |
| | | | | predicted | | |
| | | | | dimerization | | |
| 18 | CD8α | CD16A-V158 | CD8α | CD8β | 4-1BB | CD3ζ |
| | | | | | (CD137) | |
| 19 | CD8α | CD16A-V158 | CD8α | C16α | 4-1BB | CD3ζ |
| | | | | | (CD137) | |
| 20 | CD8α | CD16A-V158 | CD8α | OX40 | 4-1BB | CD3ζ |
| | | | | (CD134) | (CD137) | |
| 21 | CD8α | CD16A-V158 | CD8α | CD3ζ | 4-1BB | CD3ζ |
| | | | | | (CD137) | |
| 22 | CD8α | CD16A-V158 | CD8α | CD3ε | 4-1BB | CD3ζ |
| | | | | | (CD137) | |
| 23 | CD8α | CD16A-V158 | CD8α | CD3γ | 4-1BB | CD3ζ |
| | | | | | (CD137) | |
| 24 | CD8α | CD16A-V158 | CD8α | CD3δ | 4-1BB | CD3ζ |
| | | | | | (CD137) | |
| 25 | CD8α | CD16A-V158 | CD8α | TCR-α | 4-1BB | CD3ζ |
| | | | | | (CD137) | |
| 26 | CD8α | CD16A-V158 | CD8α | CD32 | 4-1BB | CD3ζ |
| | | | | | (CD137) | |
| 27 | CD8α | CD16A-V158 | CD8α | CD64 | 4-1BB | CD3ζ |
| | | | | | (CD137) | |
| 28 | CD8α | CD16A-V158 | CD8α | VEGFR2 | 4-1BB | CD3ζ |
| | | | | | (CD137) | |
| 29 | CD8α | CD16A-V158 | CD8α | FAS | 4-1BB | CD3ζ |
| | | | | | (CD137) | |
| 30 | CD8α | CD16A-V158 | CD8α | FGFR2B | 4-1BB | CD3ζ |
| | | | | | (CD137) | |
| 31 | CD8α | CD16A-F158 | CD8α | CD8α | 4-1BB | CD3ζ |
| | | | | | (CD137) | |
| 32 | CD8α | CD64A | CD8α | CD8α | 4-1BB | CD3ζ |
| | | | | | (CD137) | |
| 33 | CD8α | CD16A-V158 | IgG1 | CD8α | 4-1BB | CD3ζ |
| | | | (hinge- | | (CD137) | |
| | | | CH2- | | | |
| | | | CH3) | | | |
| 34 | CD8α | CD16A-V158 | IgG1 | CD8α | 4-1BB | CD3ζ |
| | | | (hinge- | | (CD137) | |
| | | | CH3) | | | |
| 35 | CD8α | CD16A-V158 | IgG1 | CD8α | 4-1BB | CD3ζ |
| | | | (hinge) | | (CD137) | |
| 36 | CD8α | CD16A-V158 | CD8- | CD8α | 4-1BB | CD3ζ |
| | | | alpha | | (CD137) | |
| | | | fragment | | | |
| | | | 1 (30 | | | |
| | | | amino | | | |
| | | | acids) | | | |
| 37 | CD8α | CD16A-V158 | CD8- | CD8α | 4-1BB | CD3ζ |
| | | | alpha | | (CD137) | |
| | | | fragment | | | |
| | | | 2 (15 | | | |
| | | | amino | | | |
| | | | acids) | | | |
| 38 | CD8α | CD16A-V158 | (Gly4Ser) × | CD8α | 4-1BB | CD3ζ |
| | | | 3 (60 | | (CD137) | |
| | | | amino | | | |
| | | | acids) | | | |
| 39 | CD8α | CD16A-V158 | (Gly4Ser) × | CD8α | 4-1BB | CD3ζ |
| | | | 6 (45 | | (CD137) | |
| | | | amino | | | |
| | | | acids) | | | |
| 40 | CD8α | CD16A-V158 | (Gly4Ser) × | CD8α | 4-1BB | CD3ζ |
| | | | 9 (30 | | (CD137) | |
| | | | amino | | | |
| | | | acids) | | | |
| 41 | CD8α | CD16A-V158 | (Gly4Ser) × | CD8α | 4-1BB | CD3ζ |
| | | | 12 (15 | | (CD137) | |
| | | | amino | | | |
| | | | acids) | | | |
| 42 | CD8α | CD16A-V158 | XTEN | CD8α | 4-1BB | CD3ζ |
| | | | (60 | | (CD137) | |
| | | | amino | | | |
| | | | acids) | | | |
| 43 | CD8α | CD16A-V158 | XTEN | CD8α | 4-1BB | CD3ζ |
| | | | (30 | | (CD137) | |
| | | | amino | | | |
| | | | acids) | | | |
| 44 | CD8α | CD16A-V158 | XTEN | CD8α | 4-1BB | CD3ζ |
| | | | (15 | | (CD137) | |
| | | | amino | | | |
| | | | acids) | | | |
| 45 | CD28 | CD16A-V158 | CD8α | CD8α | 4-1BB | CD3ζ |
| | | | | | (CD137) | |
| 46 | Murine | CD16A-V158 | CD8α | CD8α | 4-1BB | CD3ζ |
| | kappa | | | | (CD137) | |
| | chain | | | | | |
| 47 | CD16 | CD16A-V158 | CD8α | CD8α | 4-1BB | CD3ζ |
| | | | | | (CD137) | |
| 48 | CD8α | CD16A-V158 | CD8α | CD8α | ICOS | CD3ζ |
| 49 | CD8α | CD16A-V158 | CD8α | CD8α | CD27 | CD3ζ |
| 50 | CD8α | CD16A-V158 | CD8α | CD8α | GITR | CD3ζ |
| 51 | CD8α | CD16A-V158 | CD8α | CD8α | HVEM | CD3ζ |
| 52 | CD8α | CD16A-V158 | CD8α | CD8α | TIM1 | CD3ζ |
| 53 | CD8α | CD16A-V158 | CD8α | CD8α | LFA1 | CD3ζ |
| | | | | | (CD11a) | |
| 54 | CD8α | CD16A-V158 | CD8α | CD8α | CD2 | CD3ζ |
| 55 | CD8α | CD16A-V158 | CD8α | FcεRIγ | 4-1BB | FcεR1γ |
| | | | | | (CD137) | |
| 56 | CD8α | CD16A-V158 | CD8α | CD8α | 4-1BB | FcεR1γ |
| | | | | | (CD137) | |
| 57 | CD8α | CD16A-V158 | CD28 | CD28 | CD28 | CD3ζ |
| | | | (e.g., | | | |
| | | | 39aa) | | | |
| 58 | CD8α | CD16A-V158 | none | CD8 | CD28 | CD3ζ |
| 59 | CD8α | CD16A-V158 | CD8 | CD8 | CD28 + | CD3ζ |
| | | | | | CD27 | |
| 60 | CD8α | CD16A-V158 | CD8 | CD8 | CD28 + | CD3ζ |
| | | | | | OX40 | |
| 61 | CD8α | CD16A-V158 | CD8 | CD8 | 4-1BB + | CD3ζ |
| | | | | | CD28 | |
| 62 | CD8α | CD16A-V158 | CD28 | CD28 | CD28 + | CD3ζ |
| | | | | | 4-1BB | |
| 63 | CD8α | CD16A-V158 | CD28 | CD28 | 4-1BB | CD3ζ |
| 64 | CD8α | CD16A-V158 | CD8 | CD8 | CD27 | CD3ζ |
| 65 | CD8α | CD16A-V158 | CD8 | CD8 | CD28 | CD3ζ |
| 66 | CD8α | CD16A-V158 | CD8 | CD8 | ICOS | CD3ζ |
| 67 | CD8α | CD16A-V158 | CD8 | CD8 | OX40 | CD3ζ |
| 68 | CD8α | CD16A-V158 | CD8 | CD8 | CD28 and | CD3ζ |
| | | | | | ICOS | |
| 69 | CD8α | CD16A-V158 | none | CD8 | 4-1BB | CD3ζ |
| 70 | CD8α | CD16A-V158 | none | CD8 | CD27 | CD3ζ |
| 71 | CD8α | CD16A-V158 | none | CD8 | ICOS | CD3ζ |
| 72 | CD8α | CD16A-V158 | none | CD8 | OX40 | CD3ζ |
| 73 | CD8α | CD16A-V158 | none | CD8 + | 4-IBB | CD3ζ |
| | | | | 4aa | | |
| 74 | CD8α | CD16A-V158 | none | CD8 + | CD28 | CD3ζ |
| | | | | 4aa | | |
| 75 | CD8α | CD16A-V158 | CD8 | CD28 | CD28 | CD3ζ |
| 76 | CD8α | CD16A-V158 | CD28 | CD28 | CD28 | CD3ζ |
| | | | (26aa) | | | |
| 77 | CD8α | CD16A-V158 | CD28 | CD28 | CD28 | CD3ζ |
| | | | (16aa) | | | |
| 78 | CD8α | CD16A-V158 | none | CD28 | CD28 | CD3ζ |
| 79 | CD8α | CD16A-V158 | CD8 | CD8 | 41BB | CD3ζ |
| 80 | CD8α | CD16A-V158 | CD28 | CD8 | CD28 | CD3ζ |
| | | | (39 aa) | | | |

Amino acid sequences of the example ACTR polypeptides are provided below (signal sequence italicized).

SEQ ID NO: 1:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 2:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIISFFLALTSTALLFLLFFLTLRFSVVKRG
KRGRKKLLYIFKQPFMRPVQTTQEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGERRRGKGHDGLYQGLSTATKDTYD
ALHMQALPPR
SEQ ID NO: 3:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDFWVLVVVGGVLACYSLLVTVAFIIFWVRSK
KRGRKKLLYIFKQPFMRPVQTTQEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGERRRGKGHDGLYQGLSTATKDTYD
ALHMQALPPR
SEQ ID NO: 4:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDLIALVTSGALLAVLGITGYFLMNRKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 5:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDLLAALLALLAALLALLAALLARSKKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 6:
MALPVTALLLPLALLLHAARPQAAAPPKAVLKLEPPWINVLQEDSVTLTCQGARSPESDSIQWFHNGNLIPT
HTQPSYRFKANNNDSGEYTCQTGQTSLSDPVHLTVLSEWLVLQTPHLEFQEGETIMLRCHSWKDKPLVKVTF
FQNGKSQKFSHLDPTFSIPQANHSHSGDYHCTGNIGYTLFSSKPVTITVQVPSMGSSSPMGTTTPAPRPPTP
APTIASQPLSLRPEACRPAAGGAVHTRGLDFACDIYIWAPLAGTCGVLLLSLVITLYCKRGRKKLLYIFKQP
FMRPVQTTQEEDGCSCRFPEEEEGGCELRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATKDTYDALHMQALPPR
SEQ ID NO: 7:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
LLHSDYMNMTPRRPGPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTATKDTYDALHMQAL
PPR
SEQ ID NO: 8:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCALYLLR
RDQRLPPDAHKPPGGGSFRTP1QEEQADAHSTLAKIRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 9:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
LLHSDYMNMTPRRPGPTRKHYQPYAPPRDFAAYRSKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQEGLYNELQKDKM
AEAYSElGMKGERRRGKGHDGLYQGLSTATKDTYDALHMQALPPR
SEQ ID NO: 10:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIYI
WAPLAGTCGVLLLSLVITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAEAYSEIGMKGERR
RGKGHDGLYQGLSTATKDTYDALHMQALPPR
SEQ ID NO: 11:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGS
PAGSPTSTEEGTSESATPESGPGTSTEPSEGSAPGSPAGSPTIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 12:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
GGHSDYMNMTPRRPGPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTATKDTYDALHMQAL
PPR
SEQ ID NO: 13:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
GGHSDYMNMTPRRPGPTRKHYQPYAPPRDFAAYRSKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
SEQ ID NO: 14:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDMALIVLGGVAGLLLFIGLGIFFCVRKRGRK
KLLYIFKQPFMRPVQTTQEEDGCSCRFPEEEEGGCELRVKFSRSADAPAYQQGQNQLYNELNLGRREEYDVL
DKRRGRDPEMGGKPRRKNPQEGLYNELQKDKMAEAYSEIGMKGERRRGKGHDGLYQGLSTATKDTYDALHMQ
ALPPR
SEQ ID NO: 15:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDMALIVLGGVAGLLLFIGLGIFFCVRRSKRS
RGGHSDYMNMTPRRPGPTRKHYQPYAPPRDFAAYRSRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 16:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDLCYILDAILFLYGIVLTLLYCRLKKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 17:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDLLLILLGVLAGVLATLAALLARSKKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 18:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDITLGLLVAGVLVLLVSLGVAIHLCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 19:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDVSFCLVMVLLFAVDTGLYFSVKTNKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 20:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDVAAILGLGLVLGLLGPLAILLALYKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 21:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDLCYLLDGIIFIYGVILTALFLRVKKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 22:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDVMSVATIVIVDICITGGLLLLVYYWSKNRK
RGRKKLLYIFKQPFMRPVQTTQEEDGCSCRFPEEEEGGCELRVKFSRSADAPAYQQGQNQLYNELNLGRREE
YDVLDKRRGRDPEMGGKPRRKNPQEGLYNELQKDKMAEAYSEIGMKGERRRGKGHDGLYQGLSTATKDTYDA
LHMQALPPR
SEQ ID NO: 23:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDGFLFAEIVSIFVLAVGVYFIAGQDKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 24:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDGIIVTDVIATLLLALGVFCFAGHETKRGRK
KLLYIFKQPFMRPVQTTQEEDGCSCRFPEEEEGGCELRVKFSRSADAPAYQQGQNQLYNELNLGRREEYDVL
DKRRGRDPEMGGKPRRKNPQEGLYNELQKDKMAEAYSEIGMKGERRRGKGHDGLYQGLSTATKDTYDALHMQ
ALPPR
SEQ ID NO: 25:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDVIGFRILLLKVAGFNLLMTLRLWKRGRKKL
LYIFKQPFMRPVQTTQEEDGCSCRFPEEEEGGCELRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTATKDTYDALHMQAL
PPR
SEQ ID NO: 26:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIIVAVVIATAVAAIVAAVVALIYCRKKRGR
KKLLYIFKQPFMRPVQTTQEEDGCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGKGHDGLYQGLSTATKDTYDALHM
QALPPR
SEQ ID NO: 27:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDVLFYLAVGIMFLVNTVLWVTIRKEKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 28:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIIILVGTAVIAMFFWLLLVIILRTKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 29:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDLGWLCLLLLPIPLIVWVKRKKRGRKKLLYI
FKQPFMRPVQTTQEEDGCSCRFPEEEEGGCELRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKDTYDALHMQALPPR
SEQ ID NO: 30:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIAIYCIGVFLIACMVVTVILCRMKKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 31:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLFGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 32:
MALPVTALLLPLALLLHAARPQVDTTKAVITLQPPWVSVFQEETVTLHCEVLHLPGSSSTQWFLNGTATQTS
TPSYRITSASVNDSGEYRCQRGLSGRSDPIQLEIHRGWLLLQVSSRVFTEGEPLALRCHAWKDKLVYNVLYY
RNGKAFKFFHWNSNLTILKTNISHNGTYHCSGMGKHRYTSAGISVTVKELFPAPVLNASVTSPLLEGNLVTL
SCETKLLLQRPGLQLYFSFYMGSKTLRGRNTSSEYQILTARREDSGLYWCEAATEDGNVLKRSPELELQVLG
LQLPTPVWFHIYIWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQEGLYNELQKDKMA
EAYSEIGMKGERRRGKGHDGLYQGLSTATKDTYDALHMQALPPR
SEQ ID NO: 33:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQEPK
SCDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVFSCSVMHEALHN
HYTQKSISISPGKIYIWAPLAGTCGVLLLSLVITLYCKRGRKKLLYIFKQPFMRPVQTTQEEDGCSCRFPEE
EEGGCELRVKFSRSADAPAYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
SEQ ID NO: 34:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQEPK
SCDKTHTCPGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGKIYIWAPLAGTCGVLLLSLVITLYCKRGR
KKLLYIFKQPFMRPVQTTQEEDGCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGKGHDGLYQGLSTATKDTYDALHM
QALPPR
SEQ ID NO: 35:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQEPK
SCDKTHTCPIYIWAPLAGTCGVLLLSLVITLYCKRGRKKLLYIFKQPFMRPVQTTQEEDGCSCRFPEEEEGG
CELRVKFSRSADAPAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
SEQ ID NO: 36:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEAFACDIYIWAPLAGTCGVLLLSLVITLYCKRGRKKLLYIFKQPFMRPVQT
TQEEDGCSCRFPEEEEGGCELRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPPR
SEQ ID NO: 37:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPFACDIYIWAPLAGTCGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
SEQ ID NO: 38:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGG
GSGGGGSGGGGSIYIWAPLAGTCGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
SEQ ID NO: 39:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGG
GSGGGGSGGGGSGGGGSGGGGSGGGGSIYIWAPLAGTCGVLLLSLVITLYCKRGRKKLLYIFKQPFMRPVQT
TQEEDGCSCRFPEEEEGGCELRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPPR
SEQ ID NO: 40:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGG
GSGGGGSGGGGSGGGGSGGGGSGGGGSGGGGSGGGGSGGGGSIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 41:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGG
GSGGGGSGGGGSGGGGSGGGGSGGGGSGGGGSGGGGSGGGGSGGGGSGGGGSGGGGSIYIWAPLAGTCGVLL
LSLVITLYCKRGRKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERRRGKGHDGLYQGL
STATKDTYDALHMQALPPR
SEQ ID NO: 42:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGS
PAGSPTSTEEGTSESATPESGPGTSTEPSEGSAPGSPAGSPTSTEEGTSTEPSEGSAIYIWAPLAGTCGVLL
LSLVITLYCKRGRKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERRRGKGHDGLYQGL
STATKDTYDALHMQALPPR
SEQ ID NO: 43:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGS
PAGSPTSTEEGTSESATPESGPGTSTEIYIWAPLAGTCGVLLLSLVITLYCKRGRKKLLYIFKQPFMRPVQT
TQEEDGCSCRFPEEEEGGCELRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPPR
SEQ ID NO: 44:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGGS
PAGSPTSTEEGTIYIWAPLAGTCGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
SEQ ID NO: 45:
MLRLLLALNLFPSIQVTGGMRTEDLPKAVVFLEPQWYRVLEKDSVTLKCQGAYSPEDNSTQWFHNESLISSQ
ASSYFIDAATVDDSGEYRCQTNLSTLSDPVQLEVHIGWLLLQAPRWVFKEEDPIHLRCHSWKNTALHKVTYL
QNGKGRKYFHHNSDFYIPKATLKDSGSYFCRGLVGSKNVSSETVNITITQGLAVSTISSFFPPGYQTTTPAP
RPPTPAPTIASQPLSLRPEACRPAAGGAVHTRGLDFACDIYIWAPLAGTCGVLLLSLVITLYCKRGRKKLLY
IFKQPFMRPVQTTQEEDGCSCRFPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATKDTYDALHMQALPP
R
SEQ ID NO: 46:
METDTLLLWVLLLWVPGSTGDGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 47:
MWQLLLPTALLLLVSAGMRTEDLPKAVVFLEPQWYRVLEKDSVTLKCQGAYSPEDNSTQWFHNESLISSQAS
SYFIDAATVDDSGEYRCQTNLSTLSDPVQLEVHIGWLLLQAPRWVFKEEDPIHLRCHSWKNTALHKVTYLQN
GKGRKYFHHNSDFYIPKATLKDSGSYFCRGLVGSKNVSSETVNITITQGLAVSTISSFFPPGYQTTTPAPRP
PTPAPTIASQPLSLRPEACRPAAGGAVHTRGLDFACDIYIWAPLAGTCGVLLLSLVITLYCKRGRKKLLYIF
KQPFMRPVQTTQEEDGCSCRFPEEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTYDALHMQALPPR
SEQ ID NO: 48:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCCWLTKK
KYSSSVHDPNGEYMFMRAVNTAKKSRLTDVTLRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKDTYDALHMQALPPR
SEQ ID NO: 49:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCQRRKYR
SNKGESPVEPAEPCRYSCPREEEGSTIPIQEDYRKPEPACSPRVKFSRSADAPAYQQGQNQLYNELNLGRRE
EYDVLDKRRGRDPEMGGKPRRKNPQEGLYNELQKDKMAEAYSEIGMKGERRRGKGHDGLYQGLSTATKDTYD
ALHMQALPPR
SEQ ID NO: 50:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCQLGLHI
WQLRSQCMWPRETQLLLEVPPSTEDARSCQFPEEERGERSAEEKGRLGDLWVRVKFSRSADAPAYQQGQNQL
YNELNLGRREEYDVLDKRRGRDPEMGGKPRRKNPQEGLYNELQKDKMAEAYSEIGMKGERRRGKGHDGLYQG
LSTATKDTYDALHMQALPPR
SEQ ID NO: 51:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCCVKRRK
PRGDVVKVIVSVQRKRQEAEGEATVIEALQAPPDVTTVAVEETIPSFTGRSPNHRVKFSRSADAPAYQQGQN
QLYNELNLGRREEYDVLDKRRGRDPEMGGKPRRKNPQEGLYNELQKDKMAEAYSEIGMKGERRRGKGHDGLY
QGLSTATKDTYDALHMQALPPR
SEQ ID NO: 52:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKKYFFK
KEVQQLSVSFSSLQIKALQNAVEKEVQAEDNIYIENSLYATDRVKFSRSADAPAYQQGQNQLYNELNLGRRE
EYDVLDKRRGRDPEMGGKPRRKNPQEGLYNELQKDKMAEAYSEIGMKGERRRGKGHDGLYQGLSTATKDTYD
ALHMQALPPR
SEQ ID NO: 53:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCYKVGFF
KRNLKEKMEAGRGVPNGIPAEDSEQLASGQEAGDPGCLKPLHEKDSESGGGKDRVKFSRSADAPAYQQGQNQ
LYNELNLGRREEYDVLDKRRGRDPEMGGKPRRKNPQEGLYNELQKDKMAEAYSEIGMKGERRRGKGHDGLYQ
GLSTATKDTYDALHMQALPPR
SEQ ID NO: 54:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRKKQR
SRRNDEELETRAHRVATEERGRKPHQIPASTPQNPATSQHPPPPPGHRSQAPSHRPPPPGHRVQHQPQKRPP
APSGTQVHQQKGPPLPRPRVQPKPPHGAAENSLSPSSNRVKFSRSADAPAYQQGQNQLYNELNLGRREEYDV
LDKRRGRDPEMGGKPRRKNPQEGLYNELQKDKMAEAYSEIGMKGERRRGKGHDGLYQGLSTATKDTYDALHM
QALPPR
SEQ ID NO: 55:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDPQLCYILDAILFLYGIVLTLLYCRLKIQVR
KAAITSYEKSDGVYTGLSTRNQETYETLKHEKPPQKRGRKKLLYIFKQPFMRPVQTTQEEDGCSCRFPEEEE
GGCEL
SEQ ID NO: 56:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRLKIQVRKAAITSYEKSDGVYTGLSTRNQETYETLK
HEKPPQ
SEQ ID NO: 57:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIEV
MYPPPYLDNEKSNGTIIHVKGKHLCPSPLFPGPSKPFWVLVVVGGVLACYSLLVTVAFIIFWVRSKRSRLLH
SDYMNMTPRRPGPTRKHYQPYAPPRDFAAYRSRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKDTYDALHMQALPPR
SEQ ID NO: 58:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIYI
WAPLAGTCGVLLLSLVITLYCRSKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYNELQKDKMAEAYSEIGMKGERRR
GKGHDGLYQGLSTATKDTYDALHMQALPPR
SEQ ID NO: 59:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
LLHSDYMNMTPRRPGPTRKHYQPYAPPRDFAAYRSQRRKYRSNKGESPVEPAEPCHYSCPREEEGSTIPIQE
DYRKPEPACSPRVKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 60:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
LLHSDYMNMTPRRPGPTRKHYQPYAPPRDFAAYRSRRDQRLPPDAHKPPGGGSFRTPIQEEQADAHSTLAKI
RVKFSRSADAPAYQQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR
SEQ ID NO: 61:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRSKRSRLLHSDYMNMTPRRPGPTRKHYQPYAPPRDF
AAYRSRVKFSRSADAPAYQQGQNQLYNELNLGRREEYDVLDKRRGRDPEMGGKPRRKNPQEGLYNELQKDKM
AEAYSElGMKGERRRGKGHDGLYQGLSTATKDTYDALHMQALPPR
SEQ ID NO: 62:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIEV
MYPPPYLDNEKSNGTIIHVKGKHLCPSPLFPGPSKPFWVLVVVGGVLACYSLLVTVAFIIFWVRSKRSRLLH
SDYMNMTPRRPGPTRKHYQPYAPPRDFAAYRSKRGRKKLLYIFKQPFMRPVQTTQEEDGCSCRFPEEEEGGC
ELRVKFSRSADAPAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 63:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIEV
MYPPPYLDNEKSNGTIIHVKGKHLCPSPLFPGPSKPFWVLVVVGGVLACYSLLVTVAFIIFWVKRGRKKLLY
IFKQPFMRPVQTTQEEDGCSCRFPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATKDTYDALHMQALPP
R
SEQ ID NO: 64:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCQRRKYR
SNKGESPVEPAEPCHYSCPREEEGSTIPIQEDYRKPEPACSPRVKFSRSADAPAYQQGQNQLYNELNLGRRE
EYDVLDKRRGRDPEMGGKPRRKNPQEGLYNELQKDKMAEAYSEIGMKGERRRGKGHDGLYQGLSTATKDTYD
ALHMQALPPR
SEQ ID NO: 65:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
LLHSDYMNMTPRRPGPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTATKDTYDALHMQAL
PPR
SEQ ID NO: 66:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKKKYSS
SVHDPNGEYMFMRAVNTAKKSRLTDVTLRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATKDTYDALHMQALPPR
SEQ ID NO: 67:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRRDQRL
PPDAHKPPGGGSFRTPIQEEQADAHSTLAKIRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTYDALHMQALPPR
SEQ ID NO: 68:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCRSKRSR
LLHSDYMNMTPRRPGPTRKHYQPYAPPRDFAAYRSKKKYSSSVHDPNGEYMFMRAVNTAKKSRLTDVTLRVK
FSRSADAPAYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 69:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIYI
WAPLAGTCGVLLLSLVITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAEAYSEIGMKGERR
RGKGHDGLYQGLSTATKDTYDALHMQALPPR
SEQ ID NO: 70:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIYI
WAPLAGTCGVLLLSLVITLYCQRRKYRSNKGESPVEPAEPCHYSCPREEEGSTIPIQEDYRKPEPACSPRVK
FSRSADAPAYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 71:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIYI
WAPLAGTCGVLLLSLVITLYCKKKYSSSVHDPNGEYMFMRAVNTAKKSRLTDVTLRVKFSRSADAPAYQQGQ
NQLYNELNLGRREEYDVLDKRRGRDPEMGGKPRRKNPQEGLYNELQKDKMAEAYSEIGMKGERRRGKGHDGL
YQGLSTATKDTYDALHMQALPPR
SEQ ID NO: 72:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIYI
WAPLAGTCGVLLLSLVITLYCRRDQRLPPDAHKPPGGGSFRTPIQEEQADAHSTLAKIRVKFSRSADAPAYQ
QGQNQLYNELNLGRREEYDVLDKRRGRDPEMGGKPRRKNPQEGLYNELQKDKMAEAYSEIGMKGERRRGKGH
DGLYQGLSTATKDTYDALHMQALPPR
SEQ ID NO: 73:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQFAC
DIYIWAPLAGTCGVLLLSLVITLYCKRGRKKLLYIFKQPFMRPVQTTQEEDGCSCRFPEEEEGGCELRVKFS
RSADAPAYQQGQNQLYNELNLGRREEYDVLDKRRGRDPEMGGKPRRKNPQEGLYNELQKDKMAEAYSEIGMK
GERRRGKGHDGLYQGLSTATKDTYDALHMQALPPR
SEQ ID NO: 74:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQFAC
DIYIWAPLAGTCGVLLLSLVITLYCRSKRSRLLHSDYMNMTPRRPGPTRKHYQPYAPPRDFAAYRSRVKFSR
SADAPAYQQGQNQLYNELNLGRREEYDVLDKRRGRDPEMGGKPRRKNPQEGLYNELQKDKMAEAYSEIGMKG
ERRRGKGHDGLYQGLSTATKDTYDALHMQALPPR
SEQ ID NO: 75:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDFWVLVVVGGVLACYSLLVTVAFIIFWVRSK
RSRLLHSDYMNMTPRRPGPTRKHYQPYAPPRDFAAYRSRVKFSRSADAPAYQQGQNQLYNELNLGRREEYDV
LDKRRGRDPEMGGKPRRKNPQEGLYNELQKDKMAEAYSEIGMKGERRRGKGHDGLYQGLSTATKDTYDALHM
QALPPR
SEQ ID NO: 76:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQKSN
GTIIHVKGKHLCPSPLFPGPSKPFWVLVVVGGVLACYSLLVTVAFIIFWVRSKRSRLLHSDYMNMTPRRPGP
TRKHYQPYAPPRDFAAYRSRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
SEQ ID NO: 77:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQGKH
LCPSPLFPGPSKPFWVLVVVGGVLACYSLLVTVAFIIFWVRSKRSRLLHSDYMNMTPRRPGPTRKHYQPYAP
PRDFAAYRSRVKFSRSADAPAYQQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR
SEQ ID NO: 78:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQFWV
LVVVGGVLACYSLLVTVAFIIFWVRSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQEGLYNELQKDKMAEAYSEIGMKGE
RRRGKGHDGLYQGLSTATKDTYDALHMQALPPR
SEQ ID NO: 79:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQTTT
PAPRPPTPAPTIASQPLSLRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYDALHMQA
LPPR
SEQ ID NO: 80:
MALPVTALLLPLALLLHAARPGMRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVNITITQGLAVSTISSFFPPGYQIEV
MYPPPYLDNEKSNGTIIHVKGKHLCPSPLFPGPSKPIYIWAPLAGTCGVLLLSLVITLYCRSKRSRLLHSDY
MNMTPRRPGPTRKHYQPYAPPRDFAAYRSRVKFSRSADAPAYQQGQNQLYNELNLGRREEYDVLDKRRGRDP
EMGGKPRRKNPQEGLYNELQKDKMAEAYSEIGMKGERRRGKGHDGLYQGLSTATKDTYDALHMQALPPR

H. Examples of CAR Polypeptides

Exemplary CAR polypeptides for use with the methods and compositions described herein may be found, for example, in the instant description and figures or as those known in the art. The CAR polypeptides described herein may comprise an extracellular domain comprising a single-chain antibody fragment (scFv) with binding affinity and specificity for an antigen of interest (e.g., those listed in Table 3 above), a transmembrane domain, and a CD3ζ cytoplasmic signaling domain. In some embodiments, the CAR polypeptides may further include one or more co-stimulatory signaling domains, one of which may be a CD28 co-stimulatory signaling domain or a 4-1BB co-stimulatory signaling domain. The CAR polypeptides are configured such that, when expressed on a host cell, the extracellular antigen-binding domain is located extracellularly for binding to a target molecule and the CD3ζ cytoplasmic signaling domain. The co-stimulatory signaling domain may be located in the cytoplasm for triggering activation and/or effector signaling.

In some embodiments, a CAR polypeptide as described herein may comprise, from N-terminus to C-terminus, the extracellular antigen binding domain, the transmembrane domain, the optional one or more co-stimulatory domains (e.g., a CD28 co-stimulatory domain, a 4-1BB co-stimulatory signaling domain, an OX40 co-stimulatory signaling domain, a CD27 co-stimulatory signaling domain, or an ICOS co-stimulatory signaling domain), and the CD3ζ cytoplasmic signaling domain.

Alternatively or in addition, the CAR polypeptides described herein may contain two or more co-stimulatory signaling domains, which may link to each other or be separated by the cytoplasmic signaling domain. The extracellular antigen binding domain, transmembrane domain, optional co-stimulatory signaling domain(s), and cytoplasmic signaling domain in a CAR polypeptide may be linked to each other directly, or via a peptide linker. In some embodiments, any of the CAR polypeptides described herein may comprise a signal sequence at the N-terminus.

Table 5 provides exemplary CAR polypeptides described herein. These exemplary constructs have, from N-terminus to C-terminus in order, the signal sequence, the antigen binding domain (e.g., a scFv fragment targeting an antigen such as a tumor antigen or a pathogenic antigen), the hinge domain, and the transmembrane, while the positions of the optional co-stimulatory domain and the cytoplasmic signaling domain can be switched.

Table 5
Exemplary Components of CAR polypeptides.

| | Extracellular | | | | |
|---|---|---|---|---|---|
| | domain | | | Co- | Cytoplasmic |
| Signal | (antigen | Hinge | Transmembrane | stimulatory | Signaling |
| Sequence | binding) | domain | domain | domain | domain |
| CD8α | scFv (e.g., | CD8 | CD8 | 4-1BB | CD3ζ |
| | anti-GPC3 | | | | |
| | scFv) | | | | |
| CD8α | scFv (e.g., | CD28 | CD28 | CD28 | CD3ζ |
| | anti-GPC3 | | | | |
| | scFv) | | | | |

Amino acid sequences of the example CAR polypeptides are provided below (signal sequence italicized).

SEQ ID NO: 97:
MALPVTALLLPLALLLHAARPDVVMTQSPLSLPVTPGEPASISCRSSQSL
VHSNRNTYLHWYLQKPGQSPQLLIYKVSNRFSGVPDRFSGSGSGTDFTLK
ISRVEAEDVGVYYCSQNTHVPPTFGQGTKLEIKRGGGGSGGGGSGGGGSQ
VQLVQSGAEVKKPGASVKVSCKASGYTFTDYEMHWVRQAPGQGLEWMGAL
DPKTGDTAYSQKFKGRVTLTADKSTSTAYMELSSLTSEDTAVYYCTRFYS
YTYWGQGTLVTVSSTTTPAPRPPTPAPTIASQPLSLRPEACRPAAGGAVH
TRGLDFACDIYIWAPLAGTCGVLLLSLVITLYCKRGRKKLLYIFKQPFMR
PVQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 98:
MALPVTALLLPLALLLHAARPDVVMTQSPLSLPVTPGEPASISCRSSQSL
VHSNRNTYLHWYLQKPGQSPQLLIYKVSNRFSGVPDRFSGSGSGTDFTLK
ISRVEAEDVGVYYCSQNTHVPPTFGQGTKLEIKRGGGGSGGGGSGGGGSQ
VQLVQSGAEVKKPGASVKVSCKASGYTFTDYEMHWVRQAPGQGLEWMGAL
DPKTGDTAYSQKFKGRVTLTADKSTSTAYMELSSLTSEDTAVYYCTRFYS
YTYWGQGTLVTVSSIEVMYPPPYLDNEKSNGTIIHVKGKHLCPSPLFPGP
SKPFWVLVVVGGVLACYSLLVTVAFIIFWVRSKRSRLLHSDYMNMTPRRP
GPTRKHYQPYAPPRDFAAYRSRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPR

### III. Hematopoietic Cells Expressing Lactate-Modulating Factors and Optionally Chimeric Receptor Polypeptides

Provided herein are genetically engineered host cells (e.g., hematopoietic cells such as HSCs and immune cells, e.g., T cells or NK cells) expressing one or more of the lactate-modulating factors (e.g., polypeptides or nucleic acids) as described herein. The genetically engineered host cells may further express a chimeric receptor polypeptide (e.g., ACTR-expressing cells, e.g., ACTR Tcells or CAR-expressing cells, e.g., CART cells) as also described herein. In some embodiments, the host cells are hematopoietic cells or a progeny thereof. In some embodiments, the hematopoietic cells can be hematopoietic stem cells. In other embodiments, the host cells are immune cells, such as T cells or NK cells. In some embodiments, the immune cells are T cells. In some embodiments, the immune cells are NK cells. In other embodiments, the immune cells can be established cell lines, for example, NK-92 cells.

In some embodiments, the genetically engineered hematopoietic cells such as HSCs or immune cells (e.g., T cells or NK cells) may co-express any of the CAR constructs such as those disclosed herein with any of the lactate-modulating factors, such as a lactate-modulating polypeptide (e.g., LDHA, MCT, or PDK1). In some embodiments, the CAR construct may comprise a co-stimulatory domain from 4-1BB or CD28 and the lactate-modulating polypeptide is LDHA, MCT (e.g., MCT1, MCT2, or MCT4), or PDK1. The CAR construct may further comprise a hinge and transmembrane domain from CD8 or CD28.

In other embodiments, the genetically engineered hematopoietic cells such as HSCs or immune cells (e.g., T cells or NK cells) may co-express any of the ACTR constructs such as those disclosed herein with any of the lactate-modulating factors, such as a lactate-modulating polypeptide (e.g., LDHA, MCT, or PDK1). In some embodiments, the ACTR construct may comprise a co-stimulatory domain from 4-1BB or CD28 and the lactate-modulating polypeptide is LDHA, MCT (e.g., MCT1, MCT2, or MCT4), or PDK1. The ACTR construct may further comprise a hinge and transmembrane domain from CD8 or CD28.

Alternatively, the genetically engineered host cells disclosed herein may not express any chimeric receptor polypeptides. In some embodiments, the genetically engineered immune cells, which may overly express one or more lactate-modulating factors (e.g., polypeptides) as disclosed herein, may be derived from tumor-infiltrating lymphocytes (TILs). Overexpression of the lactate-modulating factors may enhance the anti-tumor activity or the TILs in tumor microenvironment. Alternatively or in addition, the genetically engineered immune cells may be T cells, which may further have genetically engineered T cell receptors. The TILs and/or genetically modified TCRs may target peptide-MHC complex, in which the peptide may be derived from a pathogen, a tumor antigen, or an auto-antigen. Some examples are provided in Table 6 below.

Any of the CAR constructs disclosed herein or an antibody to be co-used with ACTR T cells may also target any of the peptide in such peptide/MHC complex.

TABLE 6
Exemplary Peptide-MHC Targets

| Targets | Indications |
|---|---|
| NY-ESO-1 | Sarcoma, MM |
| MAGE-A10 | NSCLC, Bladder, HNSCC |
| MAGE-A4 | Sarcomas, others |
| PMEL | Melanoma |
| WT-1 | Ovarian |
| AFP | HCC |
| HPV-16 E6 | Cervical |
| HPV-16 E7 | Cervical |

In some embodiments, the host cells are immune cells, such as T cells or NK cells. In some embodiments, the immune cells are T cells. For example, the T cells can be CD4+ helper cells or CD8+ cytotoxic cells, or a combination thereof. Alternatively or in addition, the T cells can be suppressive T cells such as T_{reg} cells. In some embodiments, the immune cells are NK cells. In other embodiments, the immune cells can be established cell lines, for example, NK-92 cells. In some examples, the immune cells can be a mixture of different types of T cells and/or NK cells as known in the art. For example, the immune cells can be a population of immune cells isolated from a suitable donor (e.g., a human patient). See disclosures below.

In some instances, the lactate-modulating factor (e.g., polypeptide or nucleic acid) to be introduced into the host cells is identical to an endogenous protein of the host cell. Introducing additional copies of the coding sequences of the lactate-modulating factor into the host cell would enhance the expression level of the polypeptide (i.e., overly expressed) as relative to the native counterpart. In some instances, the lactate-modulating factor to be introduced into the host cells is heterologous to the host cell, i.e., does not exist or is not expressed in the host cell. Such a heterologous lactate-modulating factor may be a naturally-occurring protein not expressed in the host cell in nature (e.g., from a different species). Alternatively, the heterologous lactate-modulating factor may be a variant of a native protein, such as those described herein. In some examples, the exogenous (i.e., not native to the host cells) copy of the coding nucleic acid may exist extrachromosomally. In other examples, the exogenous copy of the coding sequence may be integrated into the chromosome of the host cell, and may be located at a site that is different from the native loci of the endogenous gene.

Such genetically engineered host cells have the capacity to have an enhanced rate of glycolysis and may, for example, have an enhanced capacity of taking glucose from the environment. Thus, these genetically engineered host cells may exhibit better growth and/or bioactivities under low glucose, low amino acid, low pH, and/or hypoxic conditions, for example in a tumor microenvironment. The genetically engineered cells, when expressing a chimeric receptor polypeptide as disclosed herein, can recognize and inhibit target cells, either directly (e.g., by CAR-expressing immune cells) or via an Fc-containing therapeutic agents such as an anti-tumor antibodies (e.g., by ACTR-expressing immune cells). Given their expected high proliferation rate, bioactivity, and/or survival rate in low glucose, low amino acid, low pH, and/or hypoxic environments (e.g., in a tumor microenvironment), the genetically engineered cells such as T cell and NK cells would be expected to have higher therapeutic efficacy relative to chimeric receptor polypeptide T cells that do not express or express a lower level or less active form of the lactate-modulating factor.

The population of immune cells can be obtained from any source, such as peripheral blood mononuclear cells (PBMCs), bone marrow, or tissues such as spleen, lymph node, thymus, stem cells, or tumor tissue. Alternatively, the immune cell population may be derived from stem cells, for example, hematopoietic stem cells and induced pluripotent stem cells (iPSCs). A source suitable for obtaining the type of host cells desired would be evident to one of skill in the art. In some embodiments, the population of immune cells is derived from PBMCs, which may be obtained from a patient (e.g., a human patient) who needs the treatment described herein. The type of host cells desired (e.g., T cells, NK cells, or T cells and NK cells) may be expanded within the population of cells obtained by co-incubating the cells with stimulatory molecules. As a non-limiting example, anti-CD3 and anti-CD28 antibodies may be used for expansion of T cells.

To construct the immune cells that express any of lactate-modulating factors and optionally the chimeric receptor polypeptide described herein, expression vectors for stable or transient expression of the lactate-modulating factor and/or the chimeric receptor polypeptide may be created via conventional methods as described herein and introduced into immune host cells. For example, nucleic acids encoding the lactate-modulating factors and/or the chimeric receptor polypeptides may be cloned into one or two suitable expression vectors, such as a viral vector or a non-viral vector in operable linkage to a suitable promoter. In some instances, each of the coding sequences for the chimeric receptor polypeptide and the lactate-modulating factor are on two separate nucleic acid molecules and can be cloned into two separate vectors, which may be introduced into suitable host cells simultaneously or sequentially. Alternatively, the coding sequences for the chimeric receptor polypeptide and the lactate-modulating factor are on one nucleic acid molecule and can be cloned into one vector. The coding sequences of the chimeric receptor polypeptide and the lactate-modulating factor may be in operable linkage to two distinct promoters such that the expression of the two polypeptides is controlled by different promoters. Alternatively, the coding sequences of the chimeric receptor polypeptide and the lactate-modulating factor may be in operably linkage to one promoter such that the expression of the two polypeptides is controlled by a single promoter. Suitable sequences may be inserted between the coding sequences of the two polypeptides so that two separate polypeptides can be translated from a single mRNA molecule. Such sequences, for example, IRES or ribosomal skipping site, are well known in the art. Additional descriptions are provided below.

The nucleic acids and the vector(s) may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the lactate-modulating factors and/or the chimeric receptor polypeptides. The synthetic linkers may contain nucleic acid sequences that correspond to a particular restriction site in the vector. The selection of expression vectors/plasmids/viral vectors would depend on the type of host cells for expression of the lactate-modulating factors and/or the chimeric receptor polypeptides, but should be suitable for integration and replication in eukaryotic cells.

A variety of promoters can be used for expression of the lactate-modulating factors and/or the chimeric receptor polypeptides described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, the human EF1-alpha promoter, or herpes simplex tk virus promoter. Additional promoters for expression of the lactate-modulating factors and/or the chimeric receptor polypeptides include any constitutively active promoter in an immune cell. Alternatively, any regulatable promoter may be used, such that its expression can be modulated within an immune cell.

Additionally, the vector may contain, for example, some or all of the following: a selectable marker gene, such as the neomycin gene or the kanamycin gene for selection of stable or transient transfectants in host cells; enhancer/promoter sequences from the immediate early gene of human CMV for high levels of transcription; intron sequences from the human EF1-alpha gene, transcription termination and RNA processing signals from SV40 for mRNA stability; SV40 polyomavirus origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase or an inducible caspase such as iCasp9), and reporter gene for assessing expression of the lactate-modulating polypeptides and/or the chimeric receptor polypeptide.

In one specific embodiment, such vectors also include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine deaminase, purine nucleoside phosphorylase, nitroreductase, and caspases such as caspase 8.

Suitable vectors and methods for producing vectors containing transgenes are well known and available in the art. Examples of the preparation of vectors for expression of lactate-modulating factors and/or chimeric receptor polypeptides can be found, for example, in US2014/0106449, herein incorporated in its entirety by reference.

Any of the vectors comprising a nucleic acid sequence that encodes a lactate-modulating factor and/or a chimeric receptor polypeptide described herein is also within the scope of the present disclosure. Such a vector, or the sequence encoding a lactate-modulating factor and/or a chimeric receptor polypeptide contained therein, may be delivered into host cells such as host immune cells by any suitable method. Methods of delivering vectors to immune cells are well known in the art and may include DNA electroporation, RNA electroporation, transfection using reagents such as liposomes, or viral transduction (e.g., retroviral transduction such as lentiviral transduction).

In some embodiments, the vectors for expression of the lactate-modulating factors and/or the chimeric receptor polypeptides are delivered to host cells by viral transduction (e.g., retroviral transduction such as lentiviral or gamma-retroviral transduction). Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; and WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984; and WO 95/00655). In some embodiments, the vectors for expression of the lactate-modulating factors and/or the chimeric receptor polypeptides are retroviruses. In some embodiments, the vectors for expression of the lactate-modulating factors and/or the chimeric receptor polypeptides are lentiviruses.

Examples of references describing retroviral transduction include Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993). International Patent Publication No. WO 95/07358 describes high efficiency transduction of primary B lymphocytes. See also WO 2016/040441A1, which is incorporated by reference herein for the purpose and subject matter referenced herein.

In examples in which the vectors encoding lactate-modulating factors and/or chimeric receptor polypeptides are introduced to the host cells using a viral vector, viral particles that are capable of infecting the immune cells and carry the vector may be produced by any method known in the art and can be found, for example in PCT Application No. WO 1991/002805A2, WO 1998/009271 A1, and U.S. Pat. No. 6,194,191. The viral particles are harvested from the cell culture supernatant and may be isolated and/or purified prior to contacting the viral particles with the immune cells.

In some embodiments, RNA molecules encoding any of the lactate-modulating factors and/or the chimeric receptor polypeptides as described herein may be prepared by a conventional method (e.g., in vitro transcription) and then introduced into suitable host cells, e.g., those described herein, via known methods, e.g., Rabinovich et al., Human Gene Therapy 17:1027-1035.

In some instances, the nucleic acid encoding a lactate-modulating factor and the nucleic acid encoding a suitable chimeric receptor polypeptide may be cloned into separate expression vectors, which may be introduced into suitable host cells concurrently or sequentially. For example, an expression vector (or an RNA molecule) for expressing the lactate-modulating factor may be introduced into host cells first and transfected host cells expressing the lactate-modulating factor may be isolated and cultured in vitro. An expression vector (or an RNA molecule) for expressing a suitable chimeric receptor polypeptide can then introduced into the host cells that express the lactate-modulating factor and transfected cells expressing both polypeptides can be isolated. In another example, expression vectors (or RNA molecules) each for expressing the lactate-modulating factor and the chimeric receptor polypeptide can be introduced into host cells simultaneously and transfected host cells expressing both polypeptides can be isolated via routine methodology.

In other instances, the nucleic acid encoding the lactate-modulating factor and the nucleic acid encoding the chimeric receptor polypeptide may be cloned into the same expression vector. Polynucleotides (including vectors in which such polynucleotides are operably linked to at least one regulatory element) for expression of the chimeric receptor polypeptide and lactate-modulating factor are also within the scope of the present disclosure. Non-limiting examples of useful vectors of the disclosure include viral vectors such as, e.g., retroviral vectors including gamma retroviral vectors and lentiviral vectors, and adeno-associated virus vectors (AAV vectors).

In some instances, the nucleic acid(s) encoding the lactate-modulating factor and/or the chimeric receptor polypeptide may be delivered into host cells via transposon. In some instances, the encoding nucleic acid(s) may be delivered into host cells via gene editing, for example, by CRISPR, TALEN, zinc-finger nuclease (ZFN), or meganucleases.

In some instances, the nucleic acid described herein may comprise two coding sequences, one encoding a chimeric receptor polypeptide as described herein, and the other encoding a polypeptide capable of modulating (e.g., enhancing) intracellular lactate concentrations (i.e., a lactate-modulating factor). The nucleic acid comprising the two coding sequences described herein may be configured such that the polypeptides encoded by the two coding sequences can be expressed as independent (and physically separate) polypeptides. To achieve this goal, the nucleic acid described herein may contain a third nucleotide sequence located between the first and second coding sequences. This third nucleotide sequence may, for example, encode a ribosomal skipping site. A ribosomal skipping site is a sequence that impairs normal peptide bond formation. This mechanism results in the translation of additional open reading frames from one messenger RNA. This third nucleotide sequence may, for example, encode a P2A, T2A, or F2A peptide (see, for example, Kim et al., PLoS One. 2011; 6(4):e18556). As a non-limiting example, an exemplary P2A peptide may have the amino acid sequence of ATNFSLLKQAGDVEENPGP SEQ ID NO.: 99.

In another embodiment, the third nucleotide sequence may encode an internal ribosome entry site (IRES). An IRES is an RNA element that allows translation initiation in an end-independent manner, also permitting the translation of additional open reading frames from one messenger RNA. Alternatively, the third nucleotide sequence may encode a second promoter controlling the expression of the second polypeptide. The third nucleotide sequence may also encode more than one ribosomal skipping sequence, IRES sequence, additional promoter sequence, or a combination thereof.

The nucleic acid may also include additional coding sequences (including, but not limited to, fourth and fifth coding sequences) and may be configured such that the polypeptides encoded by the additional coding sequences are expressed as further independent and physically separate polypeptides. To this end, the additional coding sequences may be separated from other coding sequences by one or more nucleotide sequences encoding one or more ribosomal skipping sequences, IRES sequences, or additional promoter sequences.

In some examples, the nucleic acid (e.g., an expression vector or an RNA molecule as described herein) may comprise coding sequences for both the lactate-modulating factor (e.g., those described herein) and a suitable chimeric receptor polypeptide, the two coding sequences, in any order, being separated by a third nucleotide sequence coding for a P2A peptide (e.g., ATNFSLLKQAGDVEENPGP; SEQ ID NO: 99). As a result, two separate polypeptides, the lactate-modulating factor and the chimeric receptor, can be produced from such a nucleic acid, wherein the P2A portion ATNFSLLKQAGDVEENPG (SEQ ID NO: 100) is linked to the upstream polypeptide (encoded by the upstream coding sequence) and residue P from the P2A peptide is linked to the downstream polypeptide (encoded by the downstream coding sequence). In some examples, the chimeric receptor polypeptide is the upstream one and the lactate-modulating factor is the downstream one. In other examples, the lactate-modulating factor is the upstream one and the chimeric receptor polypeptide is the downstream one.

In some examples, the nucleic acid (e.g., an expression vector or an RNA molecule as described herein) may comprise coding sequences for both the lactate-modulating factor (e.g., those described herein) and a suitable ACTR polypeptide, the two coding sequences, in any order, being separated by a third nucleotide sequence coding for a P2A peptide (e.g., ATNFSLLKQAGDVEENPGP; SEQ ID NO:99). As a result, two separate polypeptides, the lactate-modulating factor and the ACTR) can be produced from such a nucleic acid, wherein the P2A portion ATNFSLLKQAGDVEENPG (SEQ ID NO:100) is linked to the upstream polypeptide (encoded by the upstream coding sequence) and residue P from the P2A peptide is linked to the downstream polypeptide (encoded by the downstream coding sequence). In some examples, the ACTR polypeptide is the upstream one and the lactate-modulating factor is the downstream one. In other examples, the lactate-modulating factor is the upstream one and the ACTR polypeptide is the downstream one.

In some examples, the nucleic acid described above may further encode a linker (e.g., a GSG linker) between two segments of the encoded sequences, for example, between the upstream polypeptide and the P2A peptide.

In specific examples, the nucleic acid described herein is configured such that it expresses two separate polypeptides in the host cell to which the nucleic acid is transfected: (i) the first polypeptide that contains, from the N-terminus to the C-terminus, a suitable CAR (e.g., SEQ ID NO: 97 or SEQ ID NO: 98), a peptide linker (e.g., the GSG linker), and the ATNFSLLKQAGDVEENPG (SEQ ID NO:100) segment derived from the P2A peptide; and (ii) a second polypeptide that contains, from the N-terminus to the C-terminus, the P residue derived from the P2A peptide and the lactate-modulating factor (e.g., any of SEQ ID NOs: 81-87).

In specific examples, the nucleic acid described herein is configured such that it expresses two separate polypeptides in the host cell to which the nucleic acid is transfected: (i) the first polypeptide that contains, from the N-terminus to the C-terminus, a suitable ACTR (e.g., any of SEQ ID NOs:1-80 described herein, for example, SEQ ID NO:1 or SEQ ID NO: 57), a peptide linker (e.g., the GSG linker), and the ATNFSLLKQAGDVEENPG (SEQ ID NO:100) segment derived from the P2A peptide; and (ii) a second polypeptide that contains, from the N-terminus to the C-terminus, the P residue derived from the P2A peptide and the lactate-modulating factor (e.g., any of SEQ ID NOs: 81-87).

In some instances, additional polypeptides of interest may also be introduced into the host immune cells.

Following introduction into the host cells a vector encoding any of the lactate-modulating factors and/or the chimeric receptor polypeptides provided herein, or the nucleic acid encoding the chimeric receptor polypeptide and/or lactate-modulating factor (e.g., an RNA molecule), the cells may be cultured under conditions that allow for expression of the lactate-modulating factor and/or the chimeric receptor polypeptide. In examples in which the nucleic acid encoding the lactate-modulating factor and/or the chimeric receptor polypeptide is regulated by a regulatable promoter, the host cells may be cultured in conditions wherein the regulatable promoter is activated. In some embodiments, the promoter is an inducible promoter and the immune cells are cultured in the presence of the inducing molecule or in conditions in which the inducing molecule is produced. Determining whether the lactate-modulating factor and/or the chimeric receptor polypeptide is expressed will be evident to one of skill in the art and may be assessed by any known method, for example, detection of the lactate-modulating factor and/or the chimeric receptor polypeptide-encoding mRNA by quantitative reverse transcriptase PCR (qRT-PCR) or detection of the lactate-modulating factor and/or the chimeric receptor polypeptide protein by methods including Western blotting, fluorescence microscopy, and flow cytometry.

Alternatively, expression of the chimeric receptor polypeptide may take place in vivo after the immune cells are administered to a subject. As used herein, the term “subject” refers to any mammal such as a human, monkey, mouse, rabbit, or domestic mammal. For example, the subject may be a primate. In a preferred embodiment, the subject is human.

Alternatively, expression of a lactate-modulating factor and/or a chimeric receptor polypeptide in any of the immune cells disclosed herein can be achieved by introducing RNA molecules encoding the lactate-modulating factors and/or the chimeric receptor polypeptides. Such RNA molecules can be prepared by in vitro transcription or by chemical synthesis. The RNA molecules can then be introduced into suitable host cells such as immune cells (e.g., T cells, NK cells, or both T cells and NK cells) by, e.g., electroporation. For example, RNA molecules can be synthesized and introduced into host immune cells following the methods described in Rabinovich et al., Human Gene Therapy, 17:1027-1035 and WO 2013/040557.

In certain embodiments, a vector(s) or RNA molecule(s) comprising the lactate-modulating factor and/or the chimeric receptor polypeptide may be introduced to the host cells or immune cells in vivo. As a non-limiting example, this may be accomplished by administering a vector or RNA molecule encoding one or more lactate-modulating factors and/or one or more chimeric receptor polypeptides described herein directly to the subject (e.g., through intravenous administration), producing host cells comprising lactate-modulating factors and/or chimeric receptor polypeptides in vivo.

Methods for preparing host cells expressing any of the lactate-modulating factors and/or the chimeric receptor polypeptides described herein may also comprise activating the host cells ex vivo. Activating a host cell means stimulating a host cell into an activated state in which the cell may be able to perform effector functions. Methods of activating a host cell will depend on the type of host cell used for expression of the lactate-modulating factors and/or chimeric receptor polypeptides. For example, T cells may be activated ex vivo in the presence of one or more molecules including, but not limited to: an anti-CD3 antibody, an anti-CD28 antibody, IL-2, phytohemoagglutinin, engineered artificial stimulatory cells or particles, or a combination thereof. The engineered artificial stimulatory cells may be artificial antigen-presenting cells as known in the art. See, e.g., Neal et al., J. Immunol. Res. Ther. 2017, 2(1):68-79 and Turtle et al., Cancer J. 2010, 16(4):374-381, the relevant disclosures of each of which are hereby incorporated by reference for the purpose and subject matter referenced herein.

In other examples, NK cells may be activated ex vivo in the presence of one or more molecules such as a 4-1BB ligand, an anti-4-1BB antibody, IL-15, an anti-IL-15 receptor antibody, IL-2, IL12, IL-21, K562 cells, and/or engineered artificial stimulatory cells or particles. In some embodiments, the host cells expressing any of the lactate-modulating factors and/or the chimeric receptor polypeptides (ACTR-/CAR- and/or lactate-modulating factor-expressing cells) described herein are activated ex vivo prior to administration to a subject. Determining whether a host cell is activated will be evident to one of skill in the art and may include assessing expression of one or more cell surface markers associated with cell activation, expression or secretion of cytokines, and cell morphology.

Methods for preparing host cells expressing any of the lactate-modulating factors and/or the chimeric receptor polypeptides described herein may comprise expanding the host cells ex vivo. Expanding host cells may involve any method that results in an increase in the number of cells expressing lactate-modulating factors and/or chimeric receptor polypeptides, for example, allowing the host cells to proliferate or stimulating the host cells to proliferate. Methods for stimulating expansion of host cells will depend on the type of host cell used for expression of the lactate-modulating factors and/or the chimeric receptor polypeptides and will be evident to one of skill in the art. In some embodiments, the host cells expressing any of the lactate-modulating factors and/or the chimeric receptor polypeptides described herein are expanded ex vivo prior to administration to a subject.

In some embodiments, the host cells expressing the lactate-modulating factors and/or the chimeric receptor polypeptides are expanded and activated ex vivo prior to administration of the cells to the subject. Host cell activation and expansion may be used to allow integration of a viral vector into the genome and expression of the gene encoding a lactate-modulating factor and/or a chimeric receptor polypeptide as described herein. If mRNA electroporation is used, no activation and/or expansion may be required, although electroporation may be more effective when performed on activated cells. In some instances, a lactate-modulating factor and/or a chimeric receptor polypeptide is transiently expressed in a suitable host cell (e.g., for 3-5 days). Transient expression may be advantageous if there is a potential toxicity and should be helpful in initial phases of clinical testing for possible side effects.

Any of the host cells expressing the lactate-modulating factors and/or the chimeric receptor polypeptides may be mixed with a pharmaceutically acceptable carrier to form a pharmaceutical composition, which is also within the scope of the present disclosure.

The phrase “pharmaceutically acceptable”, as used in connection with compositions of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

The pharmaceutical compositions of the disclosure may also contain one or more additional active compounds as necessary for the particular indication being treated, preferably those with complementary activities that do not adversely affect each other. Non-limiting examples of possible additional active compounds include, e.g., IL-2 as well as various agents known in the field and listed in the discussion of combination treatments, below.

### IV. Immunotherapy Using the Genetically Engineered Hematopoietic Cells Described Herein

The genetically-engineered hematopoietic cells (e.g., hematopoietic stem cells, immune cells, such as NK cells or T cells) disclosed herein may be used in immunotherapy against various disorders, for example, cancer, infectious diseases, and autoimmune diseases.

(a) Combined Immunotherapy of Genetically Engineered Hematopoietic Cells Expressing ACTR Polypeptides and Fc-Containing Therapeutic Agents

The exemplary ACTR polypeptides of the present disclosure confer antibody-dependent cell cytotoxicity (ADCC) capacity to T lymphocytes and enhance ADCC in NK cells. When the receptor is engaged by an antibody bound to cells, it triggers T-cell activation, sustained proliferation and specific cytotoxicity against the bound cells.

The degree of affinity of CD16 for the Fc portion of Ig is a critical determinant of ADCC and thus to clinical responses to antibody immunotherapy. The CD16 with the V158 polymorphism which has a higher binding affinity for Ig and mediates superior ADCC relative to CD16 with the F158 polymorphism was selected as an example. Although the F158 receptor has lower potency than the V158 receptor in induction of T cell proliferation and ADCC, the F158 receptor may have lower in vivo toxicity than the V158 receptor making it useful in some clinical contexts.

The lactate-modulating factors to be co-expressed with ACTR polypeptides in immune cells would facilitate cell-based immune therapy such as T-cell therapy or NK-cell therapy by allowing the cells to grow and/or function effectively in a low glucose, low amino acid, low pH, and/or hypoxic environment. Antibody-directed cytotoxicity could be stopped whenever required by simple withdrawal of antibody administration. Clinical safety can be further enhanced by using mRNA electroporation to express the lactate-modulating polypeptides and/or the ACTR polypeptides transiently, to limit any potential autoimmune reactivity.

Thus, in one embodiment, the disclosure provides a method for enhancing efficacy of an antibody-based immunotherapy of a cancer in a subject in need thereof, which subject is being treated with an Fc-containing therapeutic agent such as a therapeutic antibody, which can bind to antigen-expressing cells. The Fc-containing therapeutic agent contains an Fc portion, for example, a human or humanized Fc portion, which can be recognized and bound by the Fc-binding portion (e.g., the extracellular domain of human CD16A) of the ACTR expressed on the engineered immune cells.

The methods described herein may comprise introducing into the subject a therapeutically effective amount an antibody and a therapeutically effective amount of the genetically engineered host cells such as hematopoietic cells, for example, immune cells (e.g., T lymphocytes or NK cells), which co-express a lactate-modulating factor and an ACTR polypeptide of the disclosure. The subject (e.g., a human patient such as a human cancer patient) has been treated or is being treating with an Fc-containing therapeutic agent specific to a target antigen. A target antigen may be any molecule that is associated with a disease or condition, including, but are not limited to, tumor antigens, pathogenic antigens (e.g., bacterial or viral), or antigens present on diseased cells, such as those described herein.

In the context of the present disclosure insofar as it relates to any of the disease conditions recited herein, the terms “treat”, “treatment”, and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

As used herein the term “therapeutically effective” applied to dose or amount refers to that quantity of a compound or pharmaceutical composition that is sufficient to result in a desired activity upon administration to a subject in need thereof. Note that when a combination of active ingredients is administered (e.g., a first pharmaceutical composition comprising an antibody, and a second pharmaceutical composition comprising a population of T lymphocytes or NK cells that express a lactate-modulating factor and/or an antibody-coupled T-cell receptor (ACTR) construct), the effective amount of the combination may or may not include amounts of each ingredient that would have been effective if administered individually. Within the context of the present disclosure, the term “therapeutically effective” refers to that quantity of a compound or pharmaceutical composition that is sufficient to delay the manifestation, arrest the progression, relieve or alleviate at least one symptom of a disorder treated by the methods of the present disclosure.

Host cells (e.g., hematopoietic cells, for example, immune cells such as T cells and NK cells) expressing lactate-modulating factors and ACTR polypeptides described herein are useful for enhancing ADCC in a subject and/or for enhancing the efficacy of an antibody-based immunotherapy and/or for enhancing growth and/or proliferation of immune cells in a low-glucose environment. In some embodiments, the subject is a mammal, such as a human, monkey, mouse, rabbit, or domestic mammal. In some embodiments, the subject is a human. In some embodiments, the subject is a human cancer patient. In some embodiments, the subject has been treated or is being treated with any of the therapeutic antibodies described herein.

To practice the method described herein, an effective amount of the host cells, for example, immune cells (e.g., NK cells and/or T lymphocytes) expressing any of the lactate-modulating factors and the ACTR polypeptides described herein and an effective amount of an antibody, or compositions thereof may be administered to a subject in need of the treatment via a suitable route, such as intravenous administration. As used herein, an effective amount refers to the amount of the respective agent (e.g., the NK cells and/or T lymphocytes expressing lactate-modulating factors, ACTR polypeptides, antibodies, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender, sex, and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject in need of treatment is a human cancer patient. In some embodiments, the subject in need of treatment suffers from one or more pathogenic infections (e.g., viral, bacterial, and/or fungal infections).

The methods of the disclosure may be used for treatment of any cancer or any pathogen. Specific non-limiting examples of cancers which can be treated by the methods of the disclosure include, for example, lymphoma, breast cancer, gastric cancer, neuroblastoma, osteosarcoma, lung cancer, skin cancer, prostate cancer, colorectal cancer, renal cell carcinoma, ovarian cancer, rhabdomyosarcoma, leukemia, mesothelioma, pancreatic cancer, head and neck cancer, retinoblastoma, glioma, glioblastoma, thyroid cancer, hepatocellular cancer, esophageal cancer, and cervical cancer. In certain embodiments, the cancer may be a solid tumor.

The methods of this disclosure may also be used for treating infectious diseases, which may be caused by bacterial infection, viral infection, or fungus infection. In such instances, the genetically engineered immune cells can be co-used with an Fc-containing therapeutic agent (e.g., an antibody) that targets a pathogenic antigen (e.g., an antigen associated with the bacterium, virus, or fungus that causes the infection). Specific non-limiting examples of pathogenic antigens include, but are not limited to, bacterial, viral, and/or fungal antigens. Some examples are provided below: influenza virus neuraminidase, hemagglutinin, or M2 protein, human respiratory syncytial virus (RSV) F glycoprotein or G glycoprotein, herpes simplex virus glycoprotein gB, gC, gD, or gE, Chlamydia MOMP or PorB protein, Dengue virus core protein, matrix protein, or glycoprotein E, measles virus hemagglutinin, herpes simplex virus type 2 glycoprotein gB, poliovirus I VP1, envelope glycoproteins of HIV 1, hepatitis B core antigen or surface antigen, diptheria toxin, Streptococcus 24M epitope, Gonococcal pilin, pseudorabies virus g50 (gpD), pseudorabies virus II (gpB), pseudorabies virus III (gpC), pseudorabies virus glycoprotein H, pseudorabies virus glycoprotein E, transmissible gastroenteritis glycoprotein 195, transmissible gastroenteritis matrix protein, or human hepatitis C virus glycoprotein E1 or E2.

In some embodiments, the immune cells are administered to a subject in an amount effective in enhancing ADCC activity by least 20% and/or by at least 2-fold, e.g., enhancing ADCC by 50%, 80%, 100%, 2-fold, 5-fold, 10-fold, 20-fold, 50-fold, 100-fold, or more.

The immune cells are co-administered with an Fc-containing therapeutic agent such as a therapeutic antibody in order to target cells expressing the antigen to which the Fc-containing therapeutic agent binds. In some embodiments, more than one Fc-containing therapeutic agents, such as more than one antibodies can be co-used with the immune cells. Antibody-based immunotherapy may be used to treat, alleviate, or reduce the symptoms of any disease or disorder for which the immunotherapy is considered useful in a subject.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof which comprise an Fc region, mutants thereof, fusion proteins comprising an antibody portion, humanized antibodies, chimeric antibodies, diabodies, single domain antibodies (e.g., nanobodies), linear antibodies, multispecific antibodies (e.g., bispecific antibodies) and any other modified configuration of the immunoglobulin molecule that comprises an antigen recognition site of the required specificity and an Fc region, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. The antibody for use in the present disclosure contains an Fc region recognizable by the co-used ACTR- and/or lactate-modulating factor-expressing immune cells. The Fc region may be a human or humanized Fc region.

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g. murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. The antibodies used herein may be glycosylated (e.g., fucosylated) or afucoslylated. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as a human. In some embodiments, amino acid modifications can be made in the variable region and/or the constant region.

The hematopoietic cells, for example, immune cells (e.g., T lymphocytes and/or NK cells) or HSCs expressing any of the lactate-modulating factors and/or the ACTR polypeptides disclosed herein may be administered to a subject who has been treated or is being treated with an Fc-containing antibody. For example, the immune cells may be administered to a human subject simultaneously with an antibody. Alternatively, the immune cells may be administered to a human subject during the course of an antibody-based immunotherapy. In some examples, the immune cells and an antibody can be administered to a human subject at least 4 hours apart, e.g., at least 12 hours apart, at least 1 day apart, at least 3 days apart, at least one week apart, at least two weeks apart, or at least one month apart.

In some embodiments, the antibodies described herein specifically bind to the corresponding target antigen or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen.

In some embodiments, an antibody as described herein has a suitable binding affinity for the target antigen (e.g., any one of the targets described herein) or antigenic epitopes thereof. The antibodies for use in the immune therapy methods described herein may bind to (e.g., specifically bind to) a target antigen of interest, or a specific region or an antigenic epitope therein. Table 3 above lists exemplary target antigens of interest and exemplary antibodies specific to such.

(b) Immunotherapy of Genetically Engineered Hematopoietic Cells Expressing CAR Polypeptides

The genetically engineered hematopoietic cells (e.g., hematopoietic stem cells, immune cells, such as T cells or natural killer cells) described herein, co-expressing a lactate-modulating factor and a CAR polypeptide can be used in immune therapy such as T-cell therapy or NK-cell therapy for inhibiting diseased cells expressing an antigen to which the CAR polypeptide targets, directly or indirectly (e.g., via a therapeutic agent conjugated to a tag to which the CAR polypeptide binds). The lactate-modulating factor co-expressed with a CAR polypeptide in immune cells would facilitate the cell-based immune therapy by allowing the cells to grow and/or function effectively in a low glucose, low amino acid, low pH, and/or a hypoxic environment, for example, in a tumor microenvironment. Clinical safety may be further enhanced by using mRNA electroporation to express the lactate-modulating factors and/or the CAR polypeptides transiently, to limit any potential non-tumor specific reactivity.

The methods described herein may comprise introducing into the subject a therapeutically effective amount of genetically engineered host cells such as hematopoietic cells, for example, immune cells (e.g., T lymphocytes or NK cells), which co-express a lactate-modulating factor and a CAR polypeptide of the disclosure. The subject (e.g., a human patient such as a human cancer patient) may additionally have been treated or is being treated with an anti-cancer or anti-infection therapy including, but not limited to, an anti-cancer therapeutic agent or anti-infection agent. The CAR has an antigen-binding domain that may bind any target antigen. Such a target antigen may be any molecule that is associated with a disease or condition, including, but are not limited to, tumor antigens, pathogenic antigens (e.g., bacterial, fungal, or viral), or antigens present on diseased cells, such as those described herein.

Host cells (e.g., hematopoietic cells, for example, immune cells such as T cells and NK cells) expressing lactate-modulating factors and CAR polypeptides described herein are useful for inhibiting cells expressing a target antigen and/or for enhancing growth and/or proliferation of immune cells in a low-glucose environment, a low amino acid environment, a low pH environment, and/or a hypoxic environment, for example, in a tumor microenvironment. In some embodiments, the subject is a mammal, such as a human, monkey, mouse, rabbit, or domestic mammal. In some embodiments, the subject is a human. In some embodiments, the subject is a human cancer patient. In some embodiments, the subject has additionally been treated or is being treated with any of the therapeutic antibodies described herein.

To practice the method described herein, an effective amount of the hematopoietic cells, for example, immune cells (NK cells and/or T lymphocytes) expressing any of the lactate-modulating factors and the CAR polypeptides described herein, or compositions thereof may be administered to a subject in need of the treatment via a suitable route, such as intravenous administration. As used herein, an effective amount refers to the amount of the respective agent (e.g., the NK cells and/or T lymphocytes expressing lactate-modulating factors, CAR polypeptides, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender, sex, and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject in need of treatment is a human cancer patient. In some embodiments, the subject in need of treatment suffers from one or more pathogenic infections (e.g., viral, bacterial, and/or fungal infections).

The methods of the disclosure may be used for treatment of any cancer or any pathogen. Specific non-limiting examples of cancers which can be treated by the methods of the disclosure include, for example, lymphoma, breast cancer, gastric cancer, neuroblastoma, osteosarcoma, lung cancer, skin cancer, prostate cancer, colorectal cancer, renal cell carcinoma, ovarian cancer, rhabdomyosarcoma, leukemia, mesothelioma, pancreatic cancer, head and neck cancer, retinoblastoma, glioma, glioblastoma, thyroid cancer, hepatocellular cancer, esophageal cancer, and cervical cancer. In certain embodiments, the cancer may be a solid tumor.

The methods of this disclosure may also be used for treating infectious diseases, which may be caused by bacterial infection, viral infection, or fungus infection. In such instances, genetically engineered immune cells expressing a CAR polypeptide specific to a pathogenic antigen, (e.g., an antigen associated with the bacterium, virus, or fungus that causes the infection) can be used to eliminate infected cells. Specific non-limiting examples of pathogenic antigens include, but are not limited to, bacterial, viral, and/or fungal antigens.

In some embodiments, the immune cells are administered to a subject in an amount effective in inhibiting cells expressing the target antigen by least 20% and/or by at least 2-fold, e.g., inhibiting cells expressing the target antigen by 50%, 80%, 100%, 2-fold, 5-fold, 10-fold, 20-fold, 50-fold, 100-fold, or more.

Additional therapeutic agents (e.g., antibody-based immunotherapeutic agents) may be used to treat, alleviate, or reduce the symptoms of any disease or disorder for which the therapeutic agent is considered useful in a subject.

The efficacy of the cell-based immunotherapy as described herein may be assessed by any method known in the art and would be evident to a skilled medical professional. For example, the efficacy of the cell-based immunotherapy may be assessed by survival of the subject or tumor or cancer burden in the subject or tissue or sample thereof. In some embodiments, the immune cells are administered to a subject in need of the treatment in an amount effective in enhancing the efficacy of an cell-based immunotherapy by at least 20% and/or by at least 2-fold, e.g., enhancing the efficacy of an antibody-based immunotherapy by 50%, 80%, 100%, 2-fold, 5-fold, 10-fold, 20-fold, 50-fold, 100-fold or more, as compared to the efficacy in the absence of the immune cells expressing the lactate-modulating factor and/or the CAR polypeptide.

In any of the compositions or methods described herein, the immune cells (e.g., NK and/or T cells) may be autologous to the subject, i.e., the immune cells may be obtained from the subject in need of the treatment, genetically engineered for expression of the lactate-modulating factors and/or the CAR polypeptides, and then administered to the same subject. In one specific embodiment, prior to re-introduction into the subject, the autologous immune cells (e.g., T lymphocytes or NK cells) are activated and/or expanded ex vivo. Administration of autologous cells to a subject may result in reduced rejection of the host cells as compared to administration of non-autologous cells.

Alternatively, the host cells are allogeneic cells, i.e., the cells are obtained from a first subject, genetically engineered for expression of the lactate-modulating factor and/or the chimeric receptor polypeptide (e.g., ACTR polypeptide or CAR polypeptide), and administered to a second subject that is different from the first subject but of the same species. For example, allogeneic immune cells may be derived from a human donor and administered to a human recipient who is different from the donor. In a specific embodiment, the T lymphocytes are allogeneic T lymphocytes in which the expression of the endogenous T cell receptor has been inhibited or eliminated. In one specific embodiment, prior to introduction into the subject, the allogeneic T lymphocytes are activated and/or expanded ex vivo. T lymphocytes can be activated by any method known in the art, e.g., in the presence of anti-CD3/CD28, IL-2, phytohemoagglutinin, engineered artificial stimulatory cells or particles, or a combination thereof.

NK cells can be activated by any method known in the art, e.g., in the presence of one or more agents selected from the group consisting of CD137 ligand protein, CD137 antibody, IL-15 protein, IL-15 receptor antibody, IL-2 protein, IL-12 protein, IL-21 protein, and K562 cell line, and/or engineered artificial stimulatory cells or particles. See, e.g., U.S. Pat. Nos. 7,435,596 and 8,026,097 for the description of useful methods for expanding NK cells. For example, NK cells used in the compositions or methods of the disclosure may be preferentially expanded by exposure to cells that lack or poorly express major histocompatibility complex I and/or II molecules and which have been genetically modified to express membrane bound IL-15 and 4-1BB ligand (CDI37L). Such cell lines include, but are not necessarily limited to, K562 [ATCC, CCL 243; Lozzio et al., Blood 45(3): 321-334 (1975); Klein et al., Int. J. Cancer 18: 421-431 (1976)1, and the Wilms tumor cell line HFWT (Fehniger et al., Int Rev Immunol 20(3-4):503-534 (2001); Harada H, et al., Exp Hematol 32(7):614-621 (2004)), the uterine endometrium tumor cell line HHUA, the melanoma cell line HMV-II, the hepatoblastoma cell line HuH-6, the lung small cell carcinoma cell lines Lu-130 and Lu-134-A, the neuroblastoma cell lines NB 19 and N1369, the embryonal carcinoma cell line from testis NEC 14, the cervix carcinoma cell line TCO-2, and the bone marrow-metastasized neuroblastoma cell line TNB 1 [Harada, et al., Jpn. J. Cancer Res 93: 313-319 (2002)]. Preferably the cell line used lacks or poorly expresses both MHC I and II molecules, such as the K562 and HFWT cell lines. A solid support may be used instead of a cell line. Such support should preferably have attached on its surface at least one molecule capable of binding to NK cells and inducing a primary activation event and/or a proliferative response or capable of binding a molecule having such an affect thereby acting as a scaffold. The support may have attached to its surface the CD137 ligand protein, a CD137 antibody, the IL-15 protein or an IL-15 receptor antibody. Preferably, the support will have IL-15 receptor antibody and CD137 antibody bound on its surface.

In one embodiment of the described compositions or methods, introduction (or re-introduction) of T lymphocytes, NK cells, or T lymphocytes and NK cells to the subject is followed by administering to the subject a therapeutically effective amount of IL-2.

In accordance with the present disclosure, patients can be treated by infusing therapeutically effective doses of immune cells such as T lymphocytes or NK cells comprising a lactate-modulating factor and/or a CAR polypeptide of the disclosure in the range of about 10⁵ to 10¹⁰ or more cells per kilogram of body weight (cells/Kg). The infusion can be repeated as often and as many times as the patient can tolerate until the desired response is achieved. The appropriate infusion dose and schedule will vary from patient to patient, but can be determined by the treating physician for a particular patient. Typically, initial doses of approximately 10⁶ cells/Kg will be infused, escalating to 10⁸ or more cells/Kg. IL-2 can be co-administered to expand infused cells. The amount of IL-2 can about 1-5×10⁶ international units per square meter of body surface.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The efficacy of the compositions or methods described herein may be assessed by any method known in the art and would be evident to a skilled medical professional. For example, the efficacy of the compositions or methods described herein may be assessed by survival of the subject or cancer or pathogen burden in the subject or tissue or sample thereof. In some embodiments, the compositions and methods described herein may be assessed based on the safety or toxicity of the therapy (e.g., administration of the immune cells expressing the lactate-modulating factors and the CAR polypeptides) in the subject, for example, by the overall health of the subject and/or the presence of adverse events or severe adverse events.

(c) Other Immunotherapies

In some embodiments, the genetically-engineered immune cells, expressing one or more of the lactate-modulating factors (e.g., LDHA or MCT such as MCT1, MCT2, or MCT4), may be derived from natural immune cells specific to diseased cells (e.g., cancer cells or pathogen infected cells). Such genetically-engineered immune cells (e.g., tumor-infiltrating lymphocytes or TILs) may not co-express any chimeric receptor polypeptide and can be used to destroy the target disease cells, e.g., cancer cells. The genetically-engineered TILs, expressing one or more lactate-modulating factors but not chimeric receptors, may be co-used with a bispecific antibody capable of binding to the target tumor cells and the TILs (BiTE).

In some embodiments, the genetically-engineered immune cells, expressing one or more of the lactate-modulating factors (e.g., LDHA or MCT such as MCT1, MCT2, or MCT4), may be T_{reg} cells. Such T_{reg} cells may co-express a chimeric receptor polypeptide as disclosed herein. Alternatively, the T_{reg} cells may not co-express any chimeric receptor polypeptide and can be used for the intended therapy.

### V. Combination Treatments

The compositions and methods described in the present disclosure may be utilized in conjunction with other types of therapy for cancer, such as chemotherapy, surgery, radiation, gene therapy, and so forth, or anti-infection therapy. Such therapies can be administered simultaneously or sequentially (in any order) with the immunotherapy according to the present disclosure. When co-administered with an additional therapeutic agent, suitable therapeutically effective dosages for each agent may be lowered due to the additive action or synergy.

In some instances, the immune cells (e.g., T lymphocytes and/or NK cells) expressing any of the lactate-modulating factors and/or the chimeric receptor polypeptides disclosed herein may be administered to a subject who has been treated or is being treated with an additional therapeutic agent (e.g., an additional anti-cancer therapeutic agent). For example, the immune cells may be administered to a human subject simultaneously with the additional therapeutic agent. Alternatively, the immune cells may be administered to a human subject before the additional therapeutic agent. Alternatively, the immune cells may be administered to a human subject after the additional therapeutic agent.

Genetically engineered immune cells (e.g., T cells or NK cells) that co-express a lactate-modulating factor and a CAR polypeptide specific to a tag can be co-used with a therapeutic agent conjugated to the tag. Via the therapeutic agent, which is capable of binding to an antigen associated with diseased cells such as tumor cells, such genetically engineered immune cells can be engaged with the diseased cells and inhibit their growth. Any of the antibodies listed in Table 1 above, or others specific to the same target antigen also listed in Table 1 can be conjugated to a suitable tag (e.g., those described herein) and be co-used with immune cells co-expressing the lactate-modulating factor and a CAR polypeptide specific to the tag.

The treatments of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.).

Non-limiting examples of other therapeutic agents useful for combination with the immunotherapy of the disclosure include: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones, and navelbine, epidipodophyllotoxins (etoposide and teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamine oxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycin, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (brefeldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); AKT inhibitors (such as MK-2206 2HCl, Perifosine (KRX-0401), GSK690693, Ipatasertib (GDC-0068), AZD5363, uprosertib, afuresertib, or triciribine); mTOR inhibitors, topoisomerase inhibitors (doxorubicin (adriamycin), amsacrine, camptothecin, daunorubicin, dactinomycin, eniposide, epirubicin, etoposide, idarubicin, mitoxantrone, topotecan, and irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors.

For examples of additional useful agents see also Physician's Desk Reference, 59.sup.th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

The administration of an additional therapeutic agent can be performed by any suitable route, including systemic administration as well as administration directly to the site of the disease (e.g., to a tumor).

In some embodiments, the method involves administering the additional therapeutic agent (e.g., an antibody) to the subject in one dose. In some embodiments, the method involves administering the additional therapeutic agent (e.g., an antibody) to the subject in multiple doses (e.g., at least 2, 3, 4, 5, 6, 7, or 8 doses). In some embodiments, the additional therapeutic agent (e.g., an antibody) is administered to the subject in multiple doses, with the first dose of the additional therapeutic agent (e.g., an antibody) administered to the subject about 1, 2, 3, 4, 5, 6, or 7 days prior to administration of the immune cells expressing the lactate-modulating factor and/or the CAR polypeptide. In some embodiments, the first dose of the additional therapeutic agent (e.g., an antibody) is administered to the subject between about 24-48 hours prior to the administration of the immune cells expressing the lactate-modulating factor and/or the CAR polypeptide. In some instances, the additional therapeutic agent can be an antibody specific to a target antigen of interest, for example, those listed in Table 1 and others that are specific to the same target.

In some embodiments, the additional therapeutic agent (e.g., an antibody) is administered to the subject prior to administration of the immune cells expressing the lactate-modulating factor and/or the CAR polypeptide and then subsequently about every two weeks. In some embodiments, the first two doses of the additional therapeutic agent (e.g., an antibody) are administered about one week (e.g., about 6, 7, 8, or 9 days) apart. In certain embodiments, the third and following doses are administered about every two weeks.

In any of the embodiments described herein, the timing of the administration of the additional therapeutic agent (e.g., an antibody) is approximate and includes three days prior to and three days following the indicated day (e.g., administration every three weeks encompasses administration on day 18, day 19, day 20, day 21, day 22, day 23, or day 24).

The efficacy of the methods described herein may be assessed by any method known in the art and would be evident to a skilled medical professional and/or those described herein. For example, the efficacy of the antibody-based immunotherapy may be assessed by survival of the subject or cancer burden in the subject or tissue or sample thereof. In some embodiments, the antibody-based immunotherapy is assessed based on the safety or toxicity of the therapy in the subject, for example by the overall health of the subject and/or the presence of adverse events or severe adverse events.

### VI. Kits for Therapeutic Use

The present disclosure also provides kits for use of the compositions described herein. For example, the present disclosure also provides kits comprising a population of immune cells (e.g., T lymphocytes or NK cells, constructed in vitro or in vivo) that express a lactate-modulating factor and optioanally a chimeric receptor polypeptide for use in inhibiting the growth of diseased cells, e.g., tumor cells and/or enhancing immune cell growth and/or proliferation in a low glucose environment, a low amino acid environment, a low-pH environment, and/or hypoxic environment, for example, in a tumor microenvironment. The kit may further comprise a therapeutic agent or a therapeutic agent conjugated to a tag (e.g., those described herein), to which the chimeric receptor polypeptide expressed on the immune cells bind. Such kits may include one or more containers comprising the population of the genetically engineered immune cells as described herein (e.g., T lymphocytes and/or NK cells), which co-express a lactate-modulating factor and a chimeric receptor polypeptide such as those described herein, and optionally a therapeutic agent or a therapeutic agent conjugated to a tag.

In some embodiments, the kit described herein comprises lactate-modulating factor-expressing and chimeric receptor polypeptide-expressing immune cells, which are expanded in vitro, and an antibody specific to a cell surface antibody that is present on activated T cells, for example, an anti-CD5 antibody, an anti-CD38 antibody or an anti-CD7 antibody. The lactate-modulating factor-expressing and chimeric receptor polypeptide-expressing immune cells may express any of the chimeric receptor polypeptide constructs known in the art or disclosed herein.

Alternatively, the kit disclosed herein may comprise a nucleic acid or a nucleic acid set as described herein, which collectively encodes any of the chimeric receptor polypeptides and any of the lactate-modulating factors as also described herein.

In some embodiments, the kit can additionally comprise instructions for use in any of the methods described herein. The included instructions may comprise a description of administration of the first and second pharmaceutical compositions to a subject to achieve the intended activity, e.g., inhibiting target cell growth in a subject, and/or enhancing the growth and/or proliferation of immune cells in a low-glucose environment, a low amino acid (e.g., a low glutamine environment) environment, a low pH environment, and/or a hypoxic environment (e.g., a low glucose, low amino acid, low pH or hyposic tumor microenvironment) . The kit may further comprise a description of selecting a subject suitable for treatment based on identifying whether the subject is in need of the treatment. In some embodiments, the instructions comprise a description of administering the population of genetically engineered immune cells and optionally a description of administering the tag-conjugated therapeutic agent.

The instructions relating to the use of the immune cells and optionally the tag-conjugated therapeutic agent as described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the second pharmaceutical composition is an antibody as described herein. At least one active agent in the first pharmaceutical composition is a population of immune cells (e.g., T lymphocytes or NK cells) that express a chimeric receptor polypeptide and a lactate-modulating polypeptide as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### Impact of Reduced Glucose Concentrations on T Cell Function

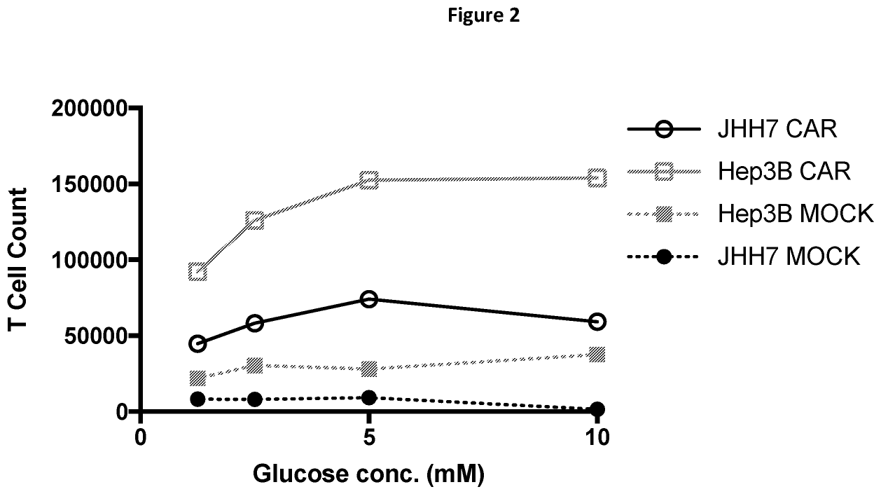

Gamma-retrovirus encoding an exemplary GPC3-targeting CAR expression construct of SEQ ID NO: 97 was generated via recombinant technology and used to infect primary human T-cells for generating cells that express a GPC3-targeting CAR polypeptide on their cell surface. A six-day flow-based proliferation assay was then used to test the functionality of the GPC3-targeting CAR expressing cells. Specifically, 200,000 untransduced mock T-cells or T-cells expressing the GPC3-targeting CAR construct were incubated together at a ratio of 4:1 (effector cells/CAR-expressing T cells to target cells) with either 50,000 GPC3+ hepatocellular carcinoma JHH7 or Hep3B tumor cells. The co-culture was incubated at 37° C. in a 5% CO₂ incubator for six days in the presence of different concentrations of glucose. At the end of six days, co-cultures were harvested and stained with an anti-CD3 antibody. The number of CD3-positive cells was evaluated by flow cytometry as a measure of T cell proliferation. At lower glucose concentrations, less CAR-T proliferation was observed (FIG. 2). These experiments demonstrate that low glucose environments may have a negative impact on CAR-T cell proliferation activity.

### Example 2

### Impact of Expressing a Lactate-Modulating Factor on T Cell Function Using a GPC3-Targeting CAR-T Expression Construct

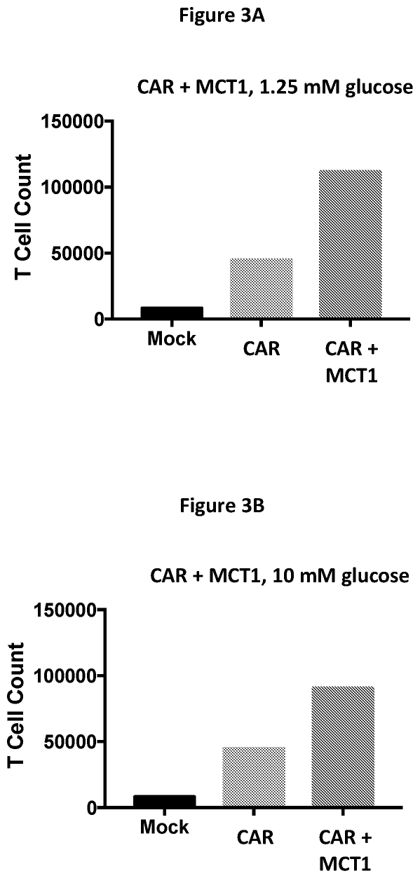

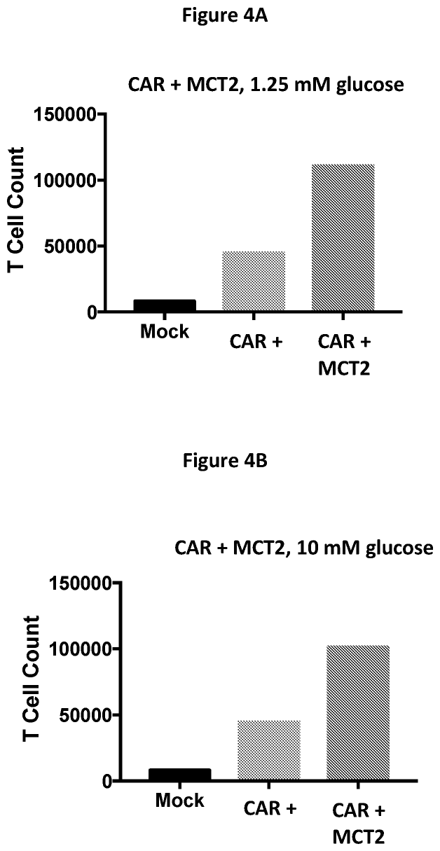

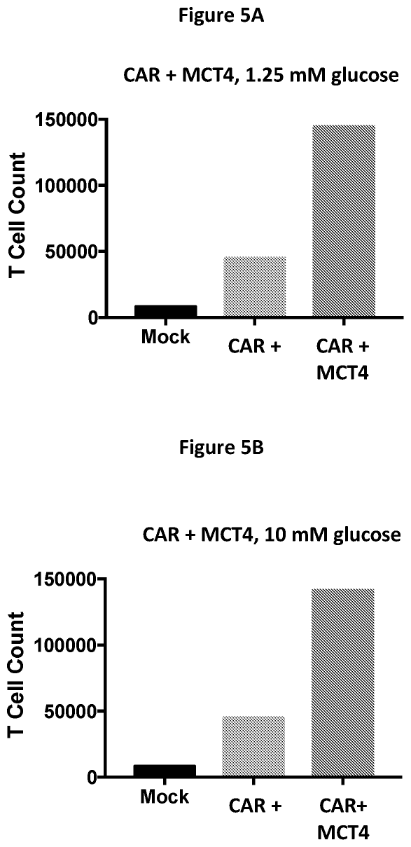

Gamma-retrovirus encoding an exemplary GPC3-targeting CAR polypeptide expression construct (SEQ ID NO: 97) was generated via recombinant technology and used to infect primary human T-cells to generate cells expressing a GPC3-targeting CAR polypeptide on their cell surface. Additionally, gamma-retroviruses encoding an exemplary GPC3-targeting CAR polypeptide (SEQ ID NO: 97 or 98) and a lactate transporting polypeptide (MCT1, MCT2, or MCT4) (SEQ ID NOs: 82-84) were generated via recombinant technology and used to infect primary human T-cells to generate cells that expressed a GPC3-targeting polypeptide and a lactate-modulating factor (e.g., a polypeptide). In the constructs encoding both the CAR polypeptide and the lactate-modulating factor, the two polypeptides were separated by a P2A ribosomal skip sequence. The variants expressed were a combination of CAR and a lactate-modulating factor as disclosed herein, for example, CAR+MCT1 (SEQ ID NO: 98 and SEQ ID NO: 82), CAR+MCT2 (SEQ ID NO: 97 and SEQ ID NO: 83), and CAR+MCT4 (SEQ ID NO: 98 and SEQ ID NO: 84). A six-day flow-based proliferation assay was then used to test the functionality of the GPC3-targeting CAR expressing cells. Specifically, 200,000 untransduced mock T-cells, T-cells expressing a GPC3-targeting CAR polypeptide, or T-cells expressing a GPC3-targeting CAR polypeptide and a lactate-modulating factor were incubated together at a ratio of 4:1 (effector cells/CAR-expressing T cells to target cells) with 50,000 GPC3+ hepatocellular carcinoma JHH7 tumor cells. The co-culture was incubated at 37° C. in a 5% CO₂ for six days in the presence of 1.25 mM glucose (tumor-relevant) and 10 mM glucose (approximate peripheral blood levels). At the end of six days, co-cultures were harvested and stained with anti-CD3 antibody. The number of CD3-positive cells was evaluated by flow cytometry as a measure of T cell proliferation. T cells expressing the lactate-modulating factor in addition to the CAR polypeptide demonstrated enhanced T cell proliferation relative to T cells expressing the CAR construct alone (FIGS. 3-5). This enhanced proliferation also occurred at tumor-relevant low glucose concentrations. These experiments demonstrated that expressing lactate-modulating factor in T cells has a positive impact on CAR-T cell proliferation activity.

### Example 3

### Impact of Expressing LDHA in Combination with an ACTR Polypeptide on T Cell Function

T cells were transduced with a virus encoding an ACTR polypeptide (SEQ ID NO: 57) and LDHA (SEQ ID NO: 81) separated by a P2A ribosomal skip sequence. T cells were cultured at a 4:1 E:T ratio with FOLRα-expressing IGROV-1 cells and a 0-20 μg/mL titration of anti-FOLRα antibody in RPMI 1640 media supplemented with 10% fetal bovine serum in a 5% CO₂ incubator at 37° C. After approximately 48 hours, supernatant samples were removed for cytokine analysis. Supernatants were analyzed for IL-2 using a homogeneous time resolved fluorescence (HTRF) assay (Cisbio) according to the manufacturer's protocol and analyzed using an EnVision Multi-label plate reader (Perkin Elmer) to detect fluorescence. The amount of IL-2 production was normalized based on the transduction efficiency of ACTR alone T cells versus cells co-expressing ACTR and LDHA. After 8 days, cultures were harvested, stained with a live/dead marker and an anti-CD3 antibody, and analyzed by flow cytometry. The number of live CD3-positive cells was used to measure T cell proliferation.

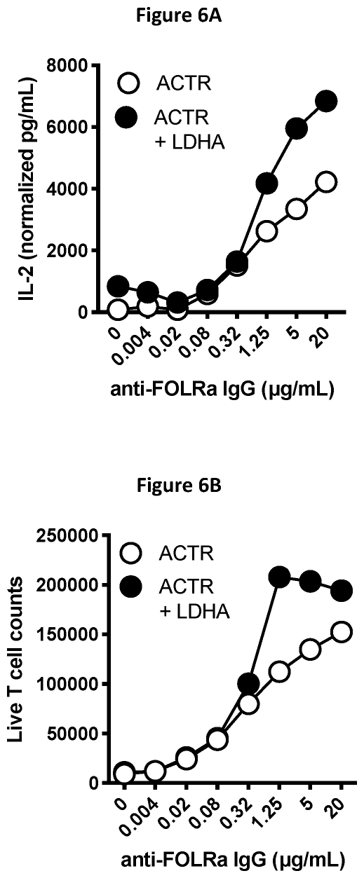

Normalized IL-2 production (FIG. 6A) and T cell proliferation (FIG. 6B) were plotted as a function of anti-FOLRα antibody concentration. These results demonstrate that T cells co-expressing ACTR and LDHA enhanced T cell function relative to T cells that expressed ACTR alone, as measured by IL-2 release or T cell proliferation in the presence of target cells and a cognate targeting antibody.

### Example 4

### T Cells Co-Expressing ACTR and LDHA Showed Enhanced Proliferation in Limited Glucose Conditions

T cells were transduced with a virus encoding an ACTR polypeptide (SEQ ID NO: 57) and LDHA (SEQ ID NO: 81) separated by a P2A ribosomal skip sequence. T cells were cultured at a 4:1 E:T ratio with FOLRα-expressing IGROV-1 cells and 5 μg/mL anti-FOLRα antibody in glucose-free RPMI 1640 media supplemented with 10% fetal bovine serum and a 0-20 mM glucose in a 5% CO₂ incubator at 37° C. After 8 days cultures were harvested, stained with a live/dead marker and an anti-CD3 antibody, and analyzed by flow cytometry. The number of live CD3-positive cells was used to measure T cell proliferation.

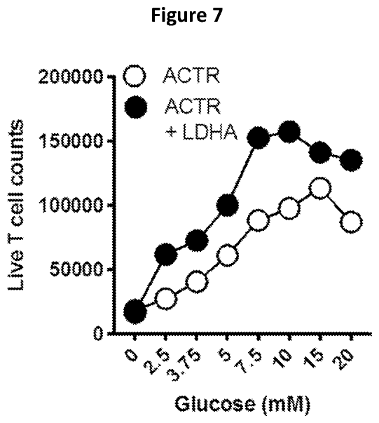

T cell proliferation was plotted as a function of glucose concentration (FIG. 7). These results demonstrate that T cells co-expressing ACTR and LDHA enhanced T cell function relative to T cells that expressed ACTR alone in limited glucose conditions, as measured by T cell proliferation in the presence of target cells and a cognate targeting antibody.

### Example 5

### T Cells Co-Expressing ACTR and LDHA Showed Enhanced Functions in the Presence of the Solid Tumor-Related Inhibitory Factor PGE₂

T cells were transduced with a virus encoding an ACTR polypeptide (SEQ ID NO: 57) and LDHA (SEQ ID NO: 81) separated by a P2A ribosomal skip sequence. T cells were cultured at a 4:1 E:T ratio with FOLRα-expressing IGROV-1 cells, 5 μg/mL of anti-FOLRα antibody, and a 0-16 μM PGE₂ in RPMI 1640 media supplemented with 10% fetal bovine serum in a 5% CO₂ incubator at 37° C.

After approximately 48 hours supernatant samples were removed for cytokine analysis. Supernatants were analyzed for IL-2 using a homogeneous time resolved fluorescence (HTRF) assay (Cisbio) according to the manufacturer's protocol, and analyzed using an EnVision Multi-label plate reader (Perkin Elmer) to detect fluorescence. The amount of IL-2 production was normalized based on the transduction efficiency of ACTR alone T cells versus cells co-expressing ACTR and LDHA. After 8 days cultures were harvested, stained with a live/dead marker and an anti-CD3 antibody, and analyzed by flow cytometry. The number of live CD3-positive cells was used to measure T cell proliferation.

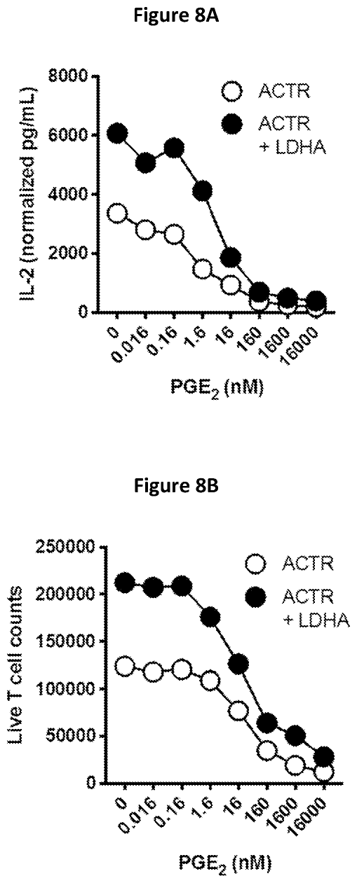

Normalized IL-2 production (FIG. 8A) or T cell proliferation (FIG. 8B) was plotted as a function of PGE₂ concentration. These results demonstrate that T cells co-expressing ACTR and LDHA enhanced T cell function relative to T cells that expressed ACTR alone when exposed to PGE₂, a well-established inhibitory factor within solid tumor microenvironments, as measured by IL-2 release or T cell proliferation in the presence of target cells and a cognate targeting antibody.

### Example 6

### T Cells Co-Expressing ACTR and LDHA Showed Enhanced IL-2 Production in the Presence of the Solid Tumor-Related Inhibitory Factor Kynurenine

T cells were transduced with a virus encoding an ACTR polypeptide (SEQ ID NO: 57) and LDHA (SEQ ID NO: 81) separated by a P2A ribosomal skip sequence. T cells were cultured at a 4:1 E:T ratio with FOLRα-expressing IGROV-1 cells, 5 μg/mL of anti-FOLRα antibody and a 0-1000 μM kynurenine in RPMI 1640 media supplemented with 10% fetal bovine serum in a 5% CO₂ incubator at 37° C. After approximately 48 hours supernatant samples were removed for cytokine analysis. Supernatants were analyzed for IL-2 using a homogeneous time resolved fluorescence (HTRF) assay (Cisbio) according to the manufacturer's protocol, and analyzed using an EnVision Multi-label plate reader (Perkin Elmer) to detect fluorescence. The amount of IL-2 production was normalized based on the transduction efficiency of ACTR alone T cells versus cells co-expressing ACTR and LDHA.

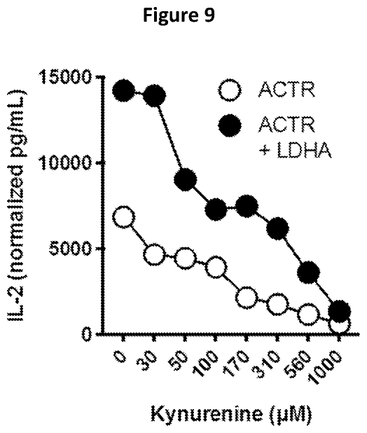

Normalized IL-2 production (FIG. 9) was plotted as a function of kynurenine concentration. These results demonstrate that T cells co-expressing ACTR and LDHA enhanced T cell function relative to T cells that expressed ACTR alone when exposed to kynurenine, a well-established inhibitory factor within solid tumor microenvironments, as measured by IL-2 release in the presence of target cells and a cognate targeting antibody.

### Example 7

### Impact of Expressing MCT1 in Combination with an ACTR Polypeptide on T Cell Function

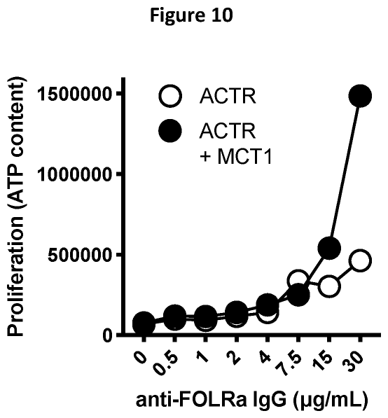

T cells were transduced with a virus encoding an ACTR polypeptide (SEQ ID NO: 57) and MCT1 (SEQ ID NO: 82) separated by a P2A ribosomal skip sequence. T cells were cultured at a 4:1 E:T ratio with FOLRα-expressing fixed OVCAR8 cells and a 0-30 μg/mL titration of anti-FOLRα antibody in RPMI 1640 media supplemented with 10% fetal bovine serum in a 5% CO₂ incubator at 37° C. After 8 days, cultures were harvested and ATP content, a measure of live cells, was determined using an ATPlite 1 step Luminescence Assay System (Perkin Elmer). The ATPlite luminescence signal, used as a measure of T cell proliferation, was analyzed according to the manufacturer's instructions using an EnVision Multi-label plate reader (Perkin Elmer) to detect luminescence. T cell proliferation (FIG. 10) was plotted as a function of anti-FOLRα antibody concentration. These results demonstrate that T cells co-expressing ACTR and MCT1 enhanced T cell function relative to T cells that expressed ACTR alone, as measured by T cell proliferation in the presence of target cells and a cognate targeting antibody.

### Example 8

### T Cells Co-Expressing ACTR and MCT1 Showed Enhanced Functions in the Presence of the Solid Tumor-Related Inhibitory Factor Kynurenine

T cells were transduced with a virus encoding an ACTR polypeptide (SEQ ID NO: 57) and MCT1 (SEQ ID NO: 82) separated by a P2A ribosomal skip sequence. T cells were cultured at a 4:1 E:T ratio with FOLRα-expressing fixed IGROV-1 cells, 1 μg/mL of anti-FOLRα antibody, and a 0-1000 μM kynurenine in RPMI 1640 media supplemented with 10% fetal bovine serum in a 5% CO₂ incubator at 37° C.

After approximately 48 hours, supernatant samples were removed for cytokine analysis. Supernatants were analyzed for IL-2 using a homogeneous time resolved fluorescence (HTRF) assay (Cisbio) according to the manufacturer's protocol, and analyzed using an EnVision Multi-label plate reader (Perkin Elmer) to detect fluorescence. The amount of IL-2 production was normalized based on the transduction efficiency of ACTR alone T cells versus cells co-expressing ACTR and MCT1.

After 7 days, half the cells were transferred to a new plate for a Cell Proliferation ELISA (Millipore Sigma) and pulsed with BrdU, incubated for approximately 16 hours in a 5% CO₂ incubator at 37° C., and analyzed for BrdU uptake following the manufacturer's instructions using an EnVision plate reader (Perkin Elmer) to detect chemiluminescence.

After 8 days the remaining half of the cells were harvested and ATP content, a measure of live cells, was determined using an ATPlite lstep Luminescence Assay System (Perkin Elmer). The ATPlite luminescence signal, used as a measure of T cell proliferation, was analyzed according to the manufacturer's instructions using an EnVision Multi-label plate reader (Perkin Elmer) to detect luminescence.

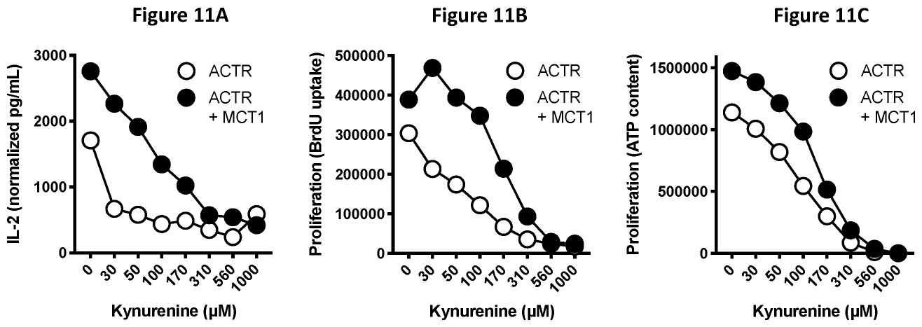

Normalized IL-2 production (FIG. 11A) or T cell proliferation as measured by BrdU uptake (FIG. 11B) or ATPlite (FIG. 11C) was plotted as a function of kynurenine concentration. These results demonstrate that T cells co-expressing ACTR and MCT1 enhanced T cell function relative to T cells that expressed ACTR alone when exposed to kynurenine, a well-established inhibitory factor within solid tumor microenvironments, as measured by IL-2 release or T cell proliferation in the presence of target cells and a cognate targeting antibody.

### Example 9

### T Cells Co-Expressing ACTR and MCT2 Showed Enhanced Proliferation in the Presence of the Solid Tumor-Related Inhibitory Factors PGE₂, TGF-β, or Kynurenine

T cells were transduced with a virus encoding an ACTR polypeptide (SEQ ID NO: 57) and MCT2 (SEQ ID NO: 83) separated by a P2A ribosomal skip sequence. T cells were cultured at a 4:1 E:T ratio with FOLRα-expressing fixed OVCAR8 cells and 1 μg/mL of anti-FOLRα antibody in RPMI 1640 media supplemented with 10% fetal bovine serum in a 5% CO₂ incubator at 37° C. Tumor-related inhibitory factors were individually added to identical T cell cultures: 0-16 μM PGE₂, 0-10 ng/ml TGF-β, or 0-1000 to 30 μM kynurenine. After 7 days the cells were harvested and ATP content, a measure of live cells, was determined using an ATPlite lstep Luminescence Assay System (Perkin Elmer). The ATPlite luminescence signal, used as a measure of T cell proliferation, was analyzed according to the manufacturer's instructions using an EnVision Multi-label plate reader (Perkin Elmer) to detect luminescence.

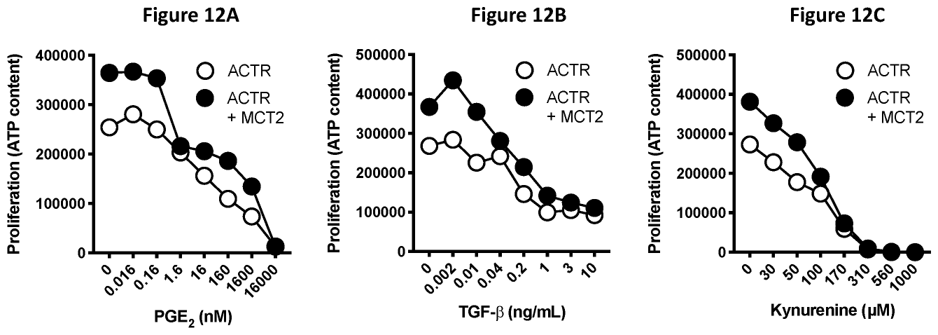

T cell proliferation as measured by ATP content was plotted as a function of PGE₂ (FIG. 12A), TGF-β (FIG. 12B), and kynurenine (FIG. 12C) concentration. These results demonstrate that T cells co-expressing ACTR and MCT2 enhanced T cell function relative to T cells that expressed ACTR alone when exposed to the well-established inhibitory factors within solid tumor microenvironments PGE₂, TGF-β, or kynurenine, as measured by T cell proliferation in the presence of target cells and a cognate targeting antibody.

### Example 10

### T Cells Co-Expressing ACTR and MCT2 Showed Enhanced Functions in the Presence of the Solid Tumor-Related Inhibitory Factor Kynurenine

T cells were transduced with a virus encoding an ACTR polypeptide (SEQ ID NO: 57) and MCT2 (SEQ ID NO: 83) separated by a P2A ribosomal skip sequence. T cells were cultured at a 4:1 E:T ratio with FOLRα-expressing fixed IGROV-1 cells, 1 μg/mL of anti-FOLRα antibody, and 0-1000 μM kynurenine, or no kynurenine, in RPMI 1640 media supplemented with 10% fetal bovine serum in a 5% CO₂ incubator at 37° C.

After approximately 48 hours, supernatant samples were removed for cytokine analysis. Supernatants were analyzed for IL-2 using a homogeneous time resolved fluorescence (HTRF) assay (Cisbio) according to the manufacturer's protocol, and analyzed using an EnVision Multi-label plate reader (Perkin Elmer) to detect fluorescence. The amount of IL-2 production was normalized based on the transduction efficiency of ACTR alone T cells versus cells co-expressing ACTR and MCT2.

After 6 days, half the cells were transferred to a new plate for a Cell Proliferation ELISA (Millipore Sigma) and pulsed with BrdU, incubated for approximately 16 hours in a 5% CO₂ incubator at 37° C., and analyzed for BrdU uptake following the manufacturer's instructions using an EnVision plate reader (Perkin Elmer) to detect chemiluminescence.

After 7 days the remaining half of the cells were harvested and ATP content, a measure of live cells, was determined using an ATPlite lstep Luminescence Assay System (Perkin Elmer). The ATPlite luminescence signal, used as a measure of T cell proliferation, was analyzed according to the manufacturer's instructions using an EnVision Multi-label plate reader (Perkin Elmer) to detect luminescence.

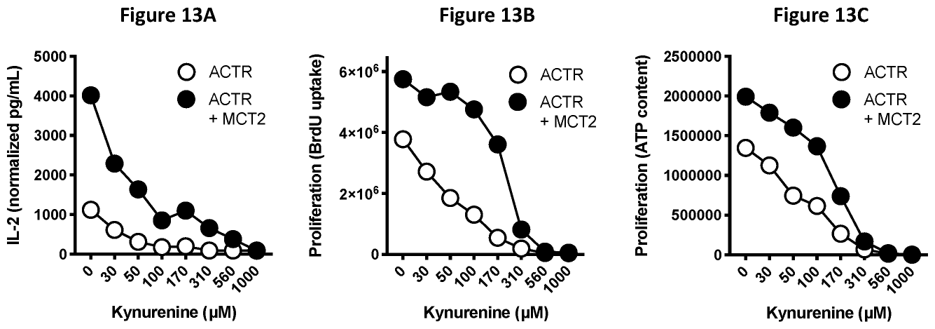

Normalized IL-2 production (FIG. 13A) and T cell proliferation, as measured by BrdU uptake (FIG. 13B) and ATPlite (FIG. 13C), were plotted as a function of kynurenine concentration. These results demonstrate that T cells co-expressing ACTR and MCT2 enhanced T cell function relative to T cells that expressed ACTR alone when exposed to kynurenine, a well-established inhibitory factor within solid tumor microenvironments, as measured by IL-2 release or T cell proliferation in the presence of target cells and a cognate targeting antibody.

### Example 11

### T Cells Co-Expressing ACTR and MCT2 Showed Enhanced IL-2 Production in the Presence of the Solid Tumor-Related Inhibitory Factor Adenosine

T cells were transduced with a virus encoding an ACTR polypeptide (SEQ ID NO: 57) and MCT2 (SEQ ID NO: 83) separated by a P2A ribosomal skip sequence. T cells were cultured at a 4:1 E:T ratio with FOLRα-expressing live or fixed IGROV-1 cells, 1 μg/mL of anti-FOLRα antibody and 0-2000 μM adenosine in RPMI 1640 media supplemented with 10% fetal bovine serum in a 5% CO₂ incubator at 37° C. After approximately 48 hours, supernatant samples were removed for cytokine analysis. Supernatants were analyzed for IL-2 using a homogeneous time resolved fluorescence (HTRF) assay (Cisbio) according to the manufacturer's protocol, and analyzed using an EnVision Multi-label plate reader (Perkin Elmer) to detect fluorescence. The amount of IL-2 production was normalized based on the transduction efficiency of ACTR alone T cells versus cells co-expressing ACTR and MCT2.

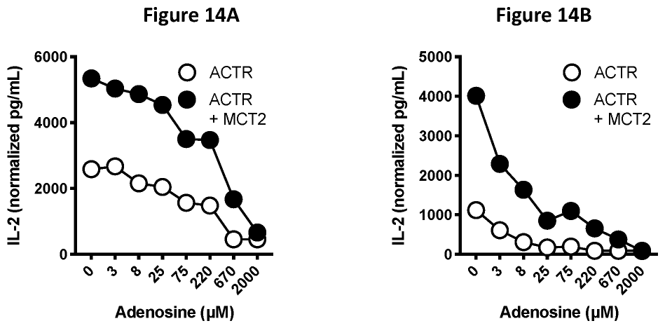

Normalized IL-2 production with live (FIG. 14A) and fixed (FIG. 14B) IGROV-1 targets was plotted as a function of adenosine concentration. These results demonstrate that T cells co-expressing ACTR and MCT2 enhanced T cell function relative to T cells that expressed ACTR alone when exposed to adenosine, a well-established inhibitory factor within solid tumor microenvironments, as measured by IL-2 release in the presence of target cells and a cognate targeting antibody.

### Example 12

### Impact of Expressing MCT4 in Combination with an ACTR Polypeptide on T Cell Function

T cells were transduced with a virus encoding an ACTR polypeptide (SEQ ID NO: 57) and MCT4 (SEQ ID NO: 84) separated by a P2A ribosomal skip sequence. T cells were cultured at a 4:1 E:T ratio with FOLRα-expressing fixed OVCAR8 cells and a 0-30 μg/mL anti-FOLRα antibody in RPMI 1640 media supplemented with 10% fetal bovine serum in a 5% CO₂ incubator at 37° C. After 8 days cultures were harvested and ATP content, a measure of live cells, was determined using an ATPlite lstep Luminescence Assay System (Perkin Elmer). The ATPlite luminescence signal, used as a measure of T cell proliferation, was analyzed according to the manufacturer's instructions using an EnVision Multi-label plate reader (Perkin Elmer) to detect luminescence.

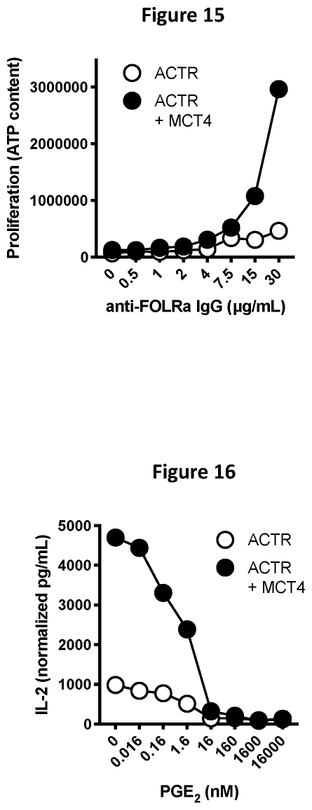

T cell proliferation (FIG. 15) was plotted as a function of anti-FOLRα antibody concentration. These results demonstrate that T cells co-expressing ACTR and MCT4 enhanced T cell function relative to T cells that expressed ACTR alone, as measured by T cell proliferation in the presence of target cells and a cognate targeting antibody.

### Example 13

### T Cells Co-Expressing ACTR and MCT4 Showed Enhanced IL-2 Production in the Presence of the Solid Tumor-Related Inhibitory Factor PGE₂

T cells were transduced with a virus encoding an ACTR polypeptide (SEQ ID NO: 57) and MCT4 (SEQ ID NO: 84) separated by a P2A ribosomal skip sequence. T cells were cultured at a 2:1 E:T ratio with FOLRα-expressing IGROV-1 cells, 5 μg/mL of anti-FOLRα antibody and 0-16 μM PGE₂ in RPMI 1640 media supplemented with 10% fetal bovine serum in a 5% CO₂ incubator at 37° C. After approximately 48 hours, supernatant samples were removed for cytokine analysis. Supernatants were analyzed for IL-2 using a homogeneous time resolved fluorescence (HTRF) assay (Cisbio) according to the manufacturer's protocol, and analyzed using an EnVision Multi-label plate reader (Perkin Elmer) to detect fluorescence. The amount of IL-2 production was normalized based on the transduction efficiency of ACTR alone T cells versus cells co-expressing ACTR and MCT4.

Normalized IL-2 production (FIG. 16) was plotted as a function of PGE₂ concentration. These results demonstrate that T cells co-expressing ACTR and MCT4 enhanced T cell function relative to T cells that expressed ACTR alone when exposed to PGE₂, a well-established inhibitory factor within solid tumor microenvironments, as measured by IL-2 release in the presence of target cells and a cognate targeting antibody.

### Example 14

### T Cells Co-Expressing ACTR and MCT4 Showed Enhanced Proliferation in the Presence of the Solid Tumor-Related Inhibitory Factor TGF-β

T cells were transduced with a virus encoding an ACTR polypeptide (SEQ ID NO: 57) and MCT4 (SEQ ID NO: 84) separated by a P2A ribosomal skip sequence. T cells were cultured at a 4:1 E:T ratio with FOLRα-expressing fixed OVCAR8 cells, 1 μg/mL of anti-FOLRα antibody, and 0-10 ng/ml TGF-β in RPMI 1640 media supplemented with 10% fetal bovine serum in a 5% CO₂ incubator at 37° C. After 8 days the cells were harvested and ATP content, a measure of live cells, was determined using an ATPlite 1step Luminescence Assay System (Perkin Elmer). The ATPlite luminescence signal, used as a measure of T cell proliferation, was analyzed according to the manufacturer's instructions using an EnVision Multi-label plate reader (Perkin Elmer) to detect luminescence.

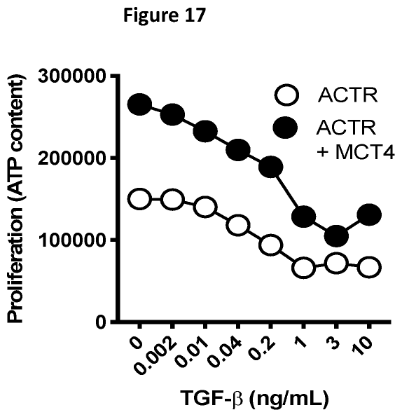

T cell proliferation as measured by ATP content was plotted as a function of TGF-β concentration (FIG. 17). These results demonstrate that T cells co-expressing ACTR and MCT4 enhanced T cell function relative to T cells that expressed ACTR alone when exposed to TGF-β, a well-established inhibitory factor within solid tumor microenvironments.

### Example 15

### T Cells Co-Expressing ACTR and MCT4 Showed Enhanced Functions in the Presence of the Solid Tumor-Related Inhibitory Factor Kynurenine

T cells were transduced with a virus encoding an ACTR polypeptide (SEQ ID NO: 57) and MCT4 (SEQ ID NO: 84) separated by a P2A ribosomal skip sequence. T cells were cultured at a 4:1 E:T ratio with FOLRα-expressing fixed IGROV-1 cells, 1 μg/mL of anti-FOLRα antibody, and 0-1000 μM kynurenine in RPMI 1640 media supplemented with 10% fetal bovine serum in a 5% CO₂ incubator at 37° C.

After approximately 48 hours, supernatant samples were removed for cytokine analysis. Supernatants were analyzed for IL-2 using a homogeneous time resolved fluorescence (HTRF) assay (Cisbio) according to the manufacturer's protocol, and analyzed using an EnVision Multi-label plate reader (Perkin Elmer) to detect fluorescence. The amount of IL-2 production was normalized based on the transduction efficiency of ACTR alone T cells versus cells co-expressing ACTR and MCT4.

After 7 days, half the cells were transferred to a new plate for a Cell Proliferation ELISA (Millipore Sigma) and pulsed with BrdU, incubated for ˜16 hours in a 5% CO₂ incubator at 37° C., and analyzed for BrdU uptake following the manufacturer's instructions using an EnVision plate reader (Perkin Elmer) to detect chemiluminescence.

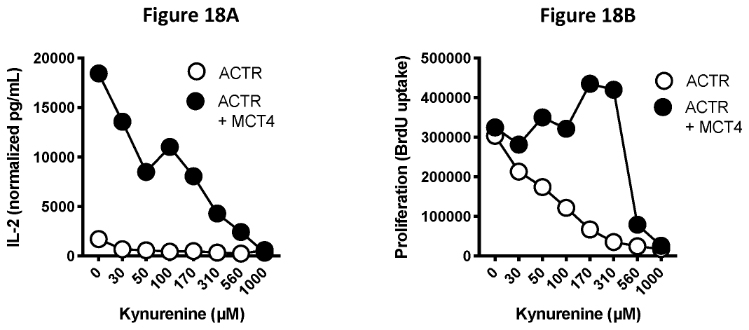

Normalized IL-2 production (FIG. 18A) and T cell proliferation, as measured by BrdU uptake (FIG. 18B), were plotted as a function of kynurenine concentration. These results demonstrate that T cells co-expressing ACTR and MCT4 enhanced T cell function relative to T cells that expressed ACTR alone when exposed to kynurenine, a well-established inhibitory factor within solid tumor microenvironments, as measured by IL-2 release or T cell proliferation in the presence of target cells and a cognate targeting antibody.

### Example 16

### Impact of Expressing a Lactate-Modulating Polypeptide on T Cell Function on Tumor Models

A lactate-modulating polypeptide transgene is co-expressed in the same T cell with a chimeric receptor polypeptide, for example, an ACTR polypeptide (e.g., SEQ ID NOs: 1-80) or a CAR polypeptide (e.g., SEQ ID NOs: 97-98). The transgene is, for example, LDHA, MCT1, MCT2, MCT4, or PDK1 (e.g., SEQ ID NOs: 81-85). The T cells are transduced with virus encoding the chimeric receptor polypeptide and the lactate-modulating polypeptide separated, for example, by a P2A ribosomal skip sequence. Transduced T cells are evaluated for anti-tumor activity in mouse tumor models. For these experiments, a tumor cell line, for example IGROV-1, is inoculated into NSG™ (NOD scid gamma, NOD.Cg-Prkdc^{scid} IL2rg^{tm1Wj1}/SzJ, Strain 005557) mice. Tumor-bearing mice are subsequently dosed with T cells expressing a chimeric receptor polypeptide alone or a chimeric receptor polypeptide and a lactate-modulating polypeptide. When the chimeric receptor polypeptide is an ACTR contruct, a tumor-targeting antibody is administered.

Tumor growth is monitored throughout the course of the experiment. T cells expressing a lactate-modulating polypeptide in addition to a chimeric receptor polypeptide (optionally with an anti-tumor antibody when the chimeric receptor polypeptide is an ACTR construct) are expected to show enhanced anti-tumor activity relative to T cells expressing the chimeric receptor polypeptide alone, for example, enhanced proliferation, enhanced T cell persistence, and/or enhanced cytokine production relative to T cells expressing the chimeric receptor polypeptide alone. Further, T cells expressing a lactate-modulating polypeptide in combination with a chimeric receptor polypeptide are also expected to show enhanced anti-cancer activites compared to T cells expressing the chimeric receptor polypeptide alone, for example, reduction in tumor growth and/or tumor formation.

In sum, the experiments disclosed in this study aim to demonstrate that expression of an exogenous lactate-modulating polypeptide in T cells, including those co-expressing a chimeric receptor polypeptide (e.g., a CAR or an ACTR) as those disclosed here would have a positive impact on T cell function and thus anti-tumor effects in vivo.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of” “only one of” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A genetically engineered hematopoietic cell, which has enhanced intracellular lactate concentrations as compared with a native hematopoietic cell of the same type.

2. The genetically engineered hematopoietic cell of claim 1, which expresses or overly expresses
(i) a lactate-modulating factor.

3. The genetically engineered hematopoietic cell of claim 2, wherein the lactate-modulating factor is a lactate-modulating polypeptide.

4. The genetically engineered hematopoietic cell of claim 3, wherein the lactate-modulating polypeptide is a monocarboxylate transporter (MCT), an enzyme involved in lactate synthesis, or a polypeptide that inhibits a pathway that competes for lactate-synthesis substrates.

5. The genetically engineered hematopoietic cell of claim 4, wherein the MCT is MCT1, MCT2, or MCT4.

6. The genetically engineered hematopoietic cell of claim 4, wherein the enzyme involved in lactate synthesis is lactate dehydrogenase A (LDHA).

7. The genetically engineered hematopoietic cell of claim 4, wherein the polypeptide that inhibits a pathway that competes for lactate-synthesis substrates is pyruvate dehydrogenase kinase 1 (PDK1).

8. The genetically engineered hematopoietic cell of any one of claims 1-7, which further expresses:
(ii) a chimeric receptor polypeptide, wherein the chimeric receptor polypeptide comprises:
(a) an extracellular target binding domain;
(b) a transmembrane domain; and
(c) a cytoplasmic signaling domain.

9. The genetically engineered hematopoietic cell of claim 8, wherein the chimeric receptor polypeptide is an antibody-coupled T cell receptor (ACTR) polypeptide, in which (a) is an extracellular Fc binding domain.

10. The genetically engineered hematopoietic cell of claim 8, wherein the chimeric receptor polypeptide is a chimeric receptor antigen (CAR) polypeptide, in which (a) is an extracellular antigen binding domain.

11. The genetically engineered hematopoietic cell of any one of claims 8-10, wherein the chimeric receptor polypeptide further comprises at least one co-stimulatory signaling domain.

12. The genetically engineered hematopoietic cell of any one of claims 8-10, wherein the chimeric receptor polypeptide, which optionally is an ACTR polypeptide, is free of co-stimulatory signaling domains.

13. The genetically engineered hematopoietic cell of any of claims 8-12, wherein the cytoplasmic signaling domain comprises an immunoreceptor tyrosine-based activation motif (ITAM).

14. The genetically engineered hematopoietic cell of any one of claims 8-13, wherein (c) is located at the C-terminus of the chimeric receptor polypeptide.

15. The genetically engineered hematopoietic cell of any one of claims 8-14, wherein the chimeric receptor polypeptide further comprises a hinge domain, which is located at the C-terminus of (a) and the N-terminus of (b).

16. The genetically engineered hematopoietic cell of any one of claims 8-15, wherein the chimeric receptor polypeptide further comprises a signal peptide at its N-terminus.

17. The genetically engineered hematopoietic cell of any one of claims 8-16, wherein the chimeric receptor polypeptide is an ACTR polypeptide, in which the extracellular target binding domain (a) is an extracellular Fc binding domain, and wherein the Fc binding domain is selected from the group consisting of:
(A) an extracellular ligand-binding domain of an Fc-receptor,
(B) an antibody fragment that binds the Fc portion of an immunoglobulin,
(C) a naturally-occurring protein that binds the Fc portion of an immunoglobulin or an Fc-binding fragment thereof, and
(D) a synthetic polypeptide that binds the Fc portion of an immunoglobulin.

18. The genetically engineered hematopoietic cell of claim 17, wherein the Fc binding domain is (A), which is an extracellular ligand-binding domain of an Fc-gamma receptor, an Fc-alpha receptor, or an Fc-epsilon receptor.

19. The genetically engineered hematopoietic cell of claim 18, wherein the Fc binding domain is an extracellular ligand-binding domain of CD16A, CD32A, or CD64A.

20. The genetically engineered hematopoietic cell of claim 18, wherein the Fc binding domain is an extracellular ligand-binding domain of F158 CD16A or V158 CD16A.

21. The genetically engineered hematopoietic cell of claim 17, wherein the Fc binding domain is (B), which is a single chain variable fragment (scFv) or a single domain antibody.

22. The genetically engineered hematopoietic cell of claim 17, wherein the Fc binding domain is (C), which is Protein A or Protein G, or an Fc-binding fragment thereof.

23. The genetically engineered hematopoietic cell of claim 17, wherein the Fc binding domain is (D), which is a Kunitz peptide, a SMIP, an avimer, an affibody, a DARPin, or an anticalin.

24. The genetically engineered hematopoietic cell of any one of claims 8-16, wherein the chimeric receptor polypeptide is a CAR polypeptide, in which the extracellular target binding domain of (a) is an antigen binding domain, and wherein the antigen binding domain is a single chain antibody fragment that binds to a tumor antigen, a pathogenic antigen, or an immune cell specific to an autoantigen.

25. The genetically engineered hematopoietic cell of claim 24, wherein the tumor antigen is associated with a hematologic tumor.

26. The genetically engineered hematopoietic cell of claim 25, wherein the tumor antigen is selected from the group consisting of CD19, CD20, CD22, Kappa-chain, CD30, CD123, CD33, LeY, CD138, CD5, BCMA, CD7, CD40, and IL-1RAP.

27. The genetically engineered hematopoietic cell of claim 24, wherein the tumor antigen is associated with a solid tumor.

28. The genetically engineered hematopoietic cell of claim 27, wherein the tumor antigen is selected from the group consisting of GD2, GPC3, FOLR, HER2, EphA2, EFGRVIII, IL13RA2, VEGFR2, ROR1, NKG2D, EpCAM, CEA, Mesothelin, MUC1, CLDN18.2, CD171, CD133, PSCA, cMET, EGFR, PSMA, FAP, CD70, MUC16, L1-CAM, and CAIX.

29. The genetically engineered hematopoietic cell of claim 24, wherein the pathogenic antigen is a bacterial antigen, a viral antigen, or a fungal antigen.

30. The genetically engineered hematopoietic cell of any one of claims 8-29, wherein the transmembrane domain of (b) is of a single-pass membrane protein.

31. The genetically engineered hematopoietic cell of claim 30, wherein the transmembrane domain is of a membrane protein selected from the group consisting of CD8α, CD8β, 4-1BB, CD28, CD34, CD4, FcεRIγ, CD16A, OX40, CD3ζ, CD3ε, CD3γ, CD38, TCRα, CD32, CD64, VEGFR2, FAS, and FGFR2B.

32. The genetically engineered hematopoietic cell of any one of claims 8-29, wherein the transmembrane domain of (b) is a non-naturally occurring hydrophobic protein segment.

33. The genetically engineered hematopoietic cell of any one of claims 8-11 and 13-32, wherein the at least one co-stimulatory signaling domain is of a co-stimulatory molecule selected from the group consisting of 4-1BB, CD28, CD28_{LL}_{_{→}}_{GG} variant, OX40, ICOS, CD27, GITR, ICOS, HVEM, TIM1, LFA1, and CD2.

34. The genetically engineered hematopoietic cell of claim 33, wherein the at least one co-stimulatory signaling domains is a CD28 co-stimulatory signaling domain or a 4-1BB co-stimulatory signaling domain.

35. The genetically engineered hematopoietic cell of any one of claims 8-11 and 13-34, wherein the chimeric receptor polypeptide comprises two co-stimulatory signaling domains.

36. The genetically engineered hematopoietic cell of claim 35, wherein the two co-stimulatory domains are:
(i) CD28 and 4-1BB; or
(ii) CD28_{LL}_{_{→}}_{GG} variant and 4-1BB.

37. The genetically engineered hematopoietic cell of claim 35, wherein one of the co-stimulatory signaling domains is a CD28 co-stimulatory signaling domain; and wherein the other co-stimulatory domain is selected from the group consisting of a 4-1BB co-stimulatory signaling domain, an OX40 co-stimulatory signaling domain, a CD27 co-stimulatory signaling domain, and an ICOS co-stimulatory signaling domain.

38. The genetically engineered hematopoietic cell of any one of claims 8-37, wherein the cytoplasmic signaling domain of (c) is a cytoplasmic domain of CD3ζ or FcεR1γ.

39. The genetically engineered hematopoietic cell of any one of claims 15-38, wherein the hinge domain is 1 to 60 amino acids in length.

40. The genetically engineered hematopoietic cell of any one of claims 15-39, wherein the hinge domain is of CD28, CD16A, CD8α, or IgG.

41. The genetically engineered hematopoietic cell of any one of claims 15-40, wherein the hinge domain is a non-naturally occurring peptide.

42. The genetically engineered hematopoietic cell of claim 41, wherein the hinge domain is an extended recombinant polypeptide (XTEN) or a (Gly₄Ser)ₙ polypeptide, in which n is an integer of 3-12, inclusive.

43. The genetically engineered hematopoietic cell of any one of claims 8-14 and 16-38, wherein the chimeric receptor polypeptide, which optionally is an ACTR polypeptide, is free of any hinge domain.

44. The genetically engineered hematopoietic cell of any one of claims 8-42, wherein the chimeric receptor, which optionally is an ACTR polypeptide, is free of a hinge domain from any non-CD16A receptor.

45. The genetically engineered hematopoietic cell of claim 17, wherein the ACTR polypeptide comprises (i) a CD28 co-stimulatory domain; and (ii) a CD28 transmembrane domain, a CD28 hinge domain, or a combination thereof.

46. The genetically engineered hematopoietic cell of claim 17, wherein the ACTR polypeptide comprises components (a)-(e) as shown in Table 4.

47. The genetically engineered hematopoietic cell of claim 17, wherein the ACTR polypeptide comprises the amino acid sequence selected from SEQ ID NOs:1-80.

48. The genetically engineered hematopoietic cell of claim 24, wherein the chimeric receptor polypeptide is a CAR polypeptide, which comprises (i) a CD28 co-stimulatory domain in combination with a CD28 transmembrane domain, a CD28 hinge domain, or a combination thereof, or (ii) a 4-1BB co-stimulatory domain in combination with a CD8 transmembrane domain, a CD8 hinge domain, or a combination thereof.

49. The genetically engineered hematopoietic cell of claim 24, wherein the CAR polypeptide comprises the amino acid sequence of SEQ ID NOs: 97 or 98.

50. The genetically engineered hematopoietic cell of any one of claims 1-49, wherein the hematopoietic is a hematopoietic stem cell or an immune cell, optionally wherein the immune cell is a natural killer cell, macrophage, neutrophil, eosinophil, or T cell.

51. The genetically engineered hematopoietic cell of claim 50, wherein the immune cell is a T cell in which the expression of an endogenous T cell receptor, an endogenous major histocompatibility complex, an endogenous beta-2-microglobulin, or a combination thereof has been inhibited or eliminated.

52. The genetically engineered hematopoietic cell of any one of claims 1-51, wherein the hematopoietic cell is an immune cell, which is derived from peripheral blood mononuclear cells (PBMC), hematopoietic stem cells (HSCs), or induced pluripotent stem cells (iPSCs).

53. The genetically engineered hematopoietic cell of any one of claims 1-52, wherein the hematopoietic cell comprises a nucleic acid or nucleic acid set, which collectively comprises:
(A) a first nucleotide sequence encoding the lactate-modulating factor; and optionally
(B) a second nucleotide sequence encoding the chimeric receptor polypeptide.

54. The genetically engineered hematopoietic cell of claim 53, wherein the nucleic acid or the nucleic acid set is an RNA molecule or a set of RNA molecules.

55. The genetically engineered hematopoietic cell of claim 53 or 54, wherein the hematopoietic cell comprises the nucleic acid, which comprises both the first nucleotide sequence and the second nucleotide sequence.

56. The genetically engineered hematopoietic cell of claim 55, wherein the nucleic acid further comprises a third nucleotide sequence located between the first nucleotide sequence and the second nucleotide sequence, wherein the third nucleotide sequence encodes a ribosomal skipping site, an internal ribosome entry site (IRES), or a second promoter.

57. The genetically engineered hematopoietic cell of claim 55, wherein the third nucleotide sequence encodes a ribosomal skipping site, which is a P2A peptide.

58. The genetically engineered hematopoietic cell of any one of claims 53-57, wherein the nucleic acid or the nucleic acid set is comprised within a vector or a set of vectors.

59. The genetically engineered hematopoietic cell of claim 58, wherein the vector or set of vectors is an expression vector or a set of expression vectors.

60. The genetically engineered hematopoietic cell of claim 58 or 59, wherein the vector or set of vectors comprises one or more viral vectors.

61. The genetically engineered hematopoietic cell of claim 60, wherein the one or more viral vectors is a retroviral vector, which optionally is a lentiviral vector or gammaretroviral vector.

62. A pharmaceutical composition, comprising a genetically engineered hematopoietic cell of any one of claims 1-61, and a pharmaceutically acceptable carrier.

63. The pharmaceutical composition of claim 62, wherein the genetically engineered hematopoietic express an ACTR polypeptide, and wherein the composition further comprises an Fc-containing therapeutic agent.

64. The pharmaceutical composition of claim 63, wherein the Fc-containing therapeutic agent is a therapeutic antibody or an Fc fusion protein.

65. The pharmaceutical composition of claim 63 or 64, wherein the Fc-containing therapeutic agent binds to a target antigen, which optionally is a tumor antigen, a pathogenic antigen, or an immune cell specific to an autoantigen.

66. The pharmaceutical composition of claim 65, wherein the pathogenic antigen is a bacterial antigen, a viral antigen, or a fungal antigen.

67. The pharmaceutical composition of claim 66, wherein the Fc-containing therapeutic agent is a therapeutic antibody selected from the group consisting of Adalimumab, Ado-Trastuzumab emtansine, Alemtuzumab, Basiliximab, Bevacizumab, Belimumab, Brentuximab, Canakinumab, Cetuximab, Certolizumab, Daclizumab, Denosumab, Dinutuximab, Eculizumab, Efalizumab, Epratuzumab, Gemtuzumab, Golimumab, hu14.18K322A, Ibritumomab, Infliximab, Ipilimumab, Labetuzumab, Muromonab, Natalizumab, Obinutuzumab, Ofatumumab, Omalizumab, Palivizumab, Panitumumab, Pertuzumab, Ramucirumab, Ranibizumab, Rituximab, Tocilizumab, Trastuzumab, Tositumomab, Ustekinumab, and Vedolizumab.

68. A kit, comprising:
a first pharmaceutical composition that comprises a genetically engineered hematopoietic cell of any one of claims 8-61, and a pharmaceutically acceptable carrier; and
a second pharmaceutical composition that comprises an Fc-containing therapeutic agent and a pharmaceutically acceptable carrier.

69. The kit of claim 68, wherein the Fc-containing therapeutic agent is an Fc fusion protein or a therapeutic antibody.

70. The kit of claim 68 or claim 69, wherein the Fc-containing therapeutic agent binds to a target antigen, which optionally is a tumor antigen, a pathogenic antigen, or an immune cell specific to an autoantigen.

71. The kit of any one of claim 70, wherein the therapeutic antibody is selected from the group consisting of Adalimumab, Ado-Trastuzumab emtansine, Alemtuzumab, Basiliximab, Bevacizumab, Belimumab, Brentuximab, Canakinumab, Certolizumab, Daclizumab, Denosumab, Dinutuximab, Eculizumab, Efalizumab, Epratuzumab, Gemtuzumab, Golimumab, hu14.18K322A, Ibritumomab, Infliximab, Ipilimumab, Labetuzumab, Muromonab, Natalizumab, Obinutuzumab, Ofatumumab, Omalizumab, Palivizumab, Panitumumab, Pertuzumab, Ramucirumab, Ranibizumab, Rituximab, Tocilizumab, Trastuzumab, Tositumomab, Ustekinumab, and Vedolizumab.

72. A method for inhibiting cells expressing a target antigen in a subject, the method comprising administering to a subject in need thereof a population of the genetically engineered hematopoietic cells set forth in any one of claims 8-61.

73. The method of claim 72, wherein the genetically engineered hematopoietic cells expressin an ACTR polypeptide, and wherein the subject has been treated or is being treating with an Fc-containing therapeutic agent specific to a target antigen.

74. The method of claim 72, wherein the genetically engineered hematopoietic cells express a CAR polypeptide, which comprises an extracelluar antigen binding domain specific to a target antigen.

75. The method of claim 73 or claim 74, wherein the target antigen is a tumor antigen, a pathogenic antigen, or an immune cell specific to an autoantigen.

76. The method of claim 75, wherein the pathogenic antigen is a bacterial antigen, a viral antigen, or a fungal antigen.

77. The method of any one of claims 73-76, wherein at least some of the cells expressing the target antigen are located in a low-glucose environment.

78. The method of any one of claims 72-77, wherein the genetically engineered hematopoietic cells are autologous.

79. The method of any one of claims 72-77, wherein the genetically engineered hematopoietic cells are allogeneic.

80. The method of any one of claims 72-79, wherein the genetically engineered hematopoietic cells are activated, expanded, or both ex vivo.

81. The method of any one of claims 73 and 75-78, wherein the Fc-containing therapeutic agent is a therapeutic antibody or an Fc fusion protein.

82. The method of claim 81, wherein the Fc-containing therapeutic agent is a therapeutic antibody selected from the group consisting of Adalimumab, Ado-Trastuzumab emtansine, Alemtuzumab, Basiliximab, Bevacizumab, Belimumab, Brentuximab, Canakinumab, Cetuximab, Certolizumab, Daclizumab, Denosumab, Dinutuximab, Eculizumab, Efalizumab, Epratuzumab, Gemtuzumab, Golimumab, hu14.18K322A, Ibritumomab, Infliximab, Ipilimumab, Labetuzumab, Muromonab, Natalizumab, Obinutuzumab, Ofatumumab, Obinutuzumab, Omalizumab, Palivizumab, Panitumumab, Pertuzumab, Ramucirumab, Ranibizumab, Rituximab, Tocilizumab, Tositumomab, Trastuzumab, Ustekinumab, and Vedolizumab.

83. The method of any one of claims 72-82, wherein the subject is a human patient suffering from a cancer and the target antigen is a tumor antigen.

84. The method of claim 83, wherein the cancer is selected from the group consisting of carcinoma, lymphoma, sarcoma, blastoma, and leukemia.

85. The method of claim 83 or claim 84, wherein the cancer is selected from the group consisting of a cancer of B-cell origin, breast cancer, gastric cancer, neuroblastoma, osteosarcoma, lung cancer, skin cancer, prostate cancer, colon cancer, renal cell carcinoma, ovarian cancer, rhabdomyosarcoma, leukemia, mesothelioma, pancreatic cancer, head and neck cancer, retinoblastoma, glioma, glioblastoma, liver cancer, and thyroid cancer.

86. The method of claim 85, wherein the cancer of B-cell origin is selected from the group consisting of B-lineage acute lymphoblastic leukemia, B-cell chronic lymphocytic leukemia, and B-cell non-Hodgkin's lymphoma.

87. The method of any one of claims 72-86, wherein the genetically engineered hematopoietic cells comprise T cells, which are activated in the presence of one or more of anti-CD3 antibody, anti-CD28 antibody, IL-2, phytohemoagglutinin, and an engineered artificial stimulatory cell or particle.

88. The method of claim 72, wherein the genetically engineered hematopoietic cells comprise natural killer cells, which are activated in the presence of one or more of 4-1BB ligand, anti-4-1BB antibody, IL-15, anti-IL-15 receptor antibody, IL-2, IL-12, IL-21, K562 cells, and an engineered artificial stimulatory cell or particle.

89. A nucleic acid or nucleic acid set, which collectively comprises:
(A) a first nucleotide sequence encoding an antibody-coupled T cell receptor (ACTR) polypeptide set forth in any one of claims 8-49; and
(B) a second nucleotide sequence encoding a lactate-modulating factor.

90. The nucleic acid or nucleic acid set, wherein the lactate-modulating factor is a lactate-modulating polypeptide.

91. The nucleic acid or nucleic acid set of claim 90, wherein the lactate-modulating polypeptide is a monocarboxylate transporter (MCT), an enzyme involved in lactate synthesis, or a polypeptide that inhibits a pathway that competes for lactate-synthesis substrates.

92. The nucleic acid or nucleic acid set of claim 91, wherein the MCT is MCT1, MCT2, or MCT4.

93. The nucleic acid or nucleic acid set of claim 91, wherein the enzyme involved in lactate synthesis is lactate dehydrogenase A (LDHA).

94. The nucleic acid of nucleic acid set of claim 91, wherein the polypeptide that inhibits a pathway that competes for lactate-synthesis substrates is pyruvate dehydrogenase kinase 1 (PDK1).

95. The nucleic acid or nucleic acid set of any one of claims 89-94, wherein the nucleic acid or the nucleic acid set is an RNA molecule or a set of RNA molecules.

96. The nucleic acid or nucleic acid set of any one of claims 89-94, wherein the nucleic acid comprises both the first nucleotide sequence and the second nucleotide sequence, and wherein the nucleic acid further comprises a third nucleotide sequence located between the first nucleotide sequence and the second nucleotide sequence, the third nucleotide sequence encoding a ribosomal skipping site, an internal ribosome entry site (IRES), or a second promoter.

97. The nucleic acid or nucleic acid set of claim 96, wherein the ribosomal skipping site is a P2A peptide.

98. The nucleic acid or nucleic acid set of any one of claims 89-97, wherein the nucleic acid or the nucleic acid set is comprised within a vector or a set of vectors.

99. The nucleic acid or nucleic acid set of claim 98, wherein the vector or set of vectors is an expression vector or a set of expression vectors.

100. The nucleic acid or nucleic acid set of claim 98 or claim 99, wherein the vector or set of vectors comprises one or more viral vectors.

101. The nucleic acid or nucleic acid set of claim 100, wherein the one or more viral vectors is a retroviral vector, which optionally is a lentiviral vector or gammaretroviral vector.

102. A method for generating modified hematopoietic cells in vivo, the method comprising administering to a subject in need thereof the nucleic acid or nucleic acid set of any one of claims 89-101.

103. The method of claim 102, further comprising administering to the subject an Fc-containing therapeutic agent specific to the target antigen.
